(19) United States
(12) Patent Application Publication
(10) Pub. No.: US 2022/0145249 A1
(43) Pub. Date: May 12, 2022

(54) A Method of Altering a Differentiation Status of a Cell

(71) Applicant: Agency for Science, Technology and Research, Singapore (SG)

(72) Inventors: Premkumar Jayaraman, Singapore (SG); Kah Weng Steve Oh, Singapore (SG)

(21) Appl. No.: 17/602,060

(22) PCT Filed: Apr. 10, 2020
(86) PCT No.: PCT/SG2020/050220

§ 371 Date: Oct. 7, 2021

(30) Foreign Application Priority Data
Apr. 11, 2019 (SG) 10201903268X

(51) Int. Cl. C12N 5/079; C12N 9/22; A61K 35/30; C12N 15/113

(52) U.S. Cl. CPC C12N 5/0621; C12N 9/22; A61K 35/30; C12N 2533/52; C12N 2506/45; C12N 2510/00; C12N 15/113

(57) ABSTRACT

The invention relates to a method of altering a differentiation status of a stem cell by modulating the expression of one or more differentiation factors with a nuclease-deactivated Cas9 (dCas9) fusion protein comprising dCas9 and a transcriptional activator. The method may further include a guide RNA (gRNA) and an activator module comprising RNA-binding protein binding capable of binding to the gRNA. In one embodiment, the dCas9 fusion protein comprises dCas9 and VP64 while the activator module comprises MS2 coat protein and p65. The one or more differentiation factors may comprise PAX6, MITF and OTX2 for differentiation of pluripotent stem cell into retinal pigment epithelium (RPE). Also disclosed are cells comprising the dCas9 fusion protein, gRNA, kits, and method of treating a disease thereof.

### TECHNICAL FIELD

The present invention relates to a cell programming. In particular, the present invention relates to a method of altering a differentiation status of a cell.

### BACKGROUND

Over the past few years there has been great progress in generating matured/specialized cell from stem cells. However, many methods known in the art for generating specialized cell rely on the use of growth factors or small molecules.

Conventionally, growth factors and cytokines are used for stem cell differentiation and other clinical applications. Most commonly, CHO or bacterial cells are used and recombinant exogenous DNA is inserted into the cells to produce growth factors and/or cytokines. However, such methods are known to have various limitations including post-translational modification problems (such as glycosylation pattern and/or folding of the protein that is not identical to those found in human), limitation of exon size, and laborious upstream processing in the process of selecting clones. The use of recombinant exogenous DNA has also been shown to lose expression over time, have low productivity, have increased risk of insertion of recombinant exogenous DNA into functional genes, and requires costly and time-consuming purification of cells from viral vectors.

Loss of expression over time in plasmid-based systems have been known to effect productivity in plasmid-based system that over time can lead to no protein production. For example, such loss of expression may be caused by two plasmids of the same sequence recombining to form a single dimeric circle of two origins of replication. Furthermore, excessive positive selection for cells with plasmid has also been known to induce structural instability, which may lead to elimination of recombinant gene. At the same time, if plasmid copy number is too high, translational efficiency may decrease and recombinant protein yields would see a reduction. Selection of bacteria with plasmid using antibiotic resistance gene in plasmid also pose a problem as it is undesirable to use antibiotic in either food or therapeutic products. Whilst it is possible to remove antibiotics, the removal process is expensive, time consuming and complex.

Another problem that may arise includes lower productivity due to a low copy number of the recombinant gene. Whilst the low copy number can be overcome by performing multiple gene integration into the chromosome to yield similar expression levels to those achieved by plasmid systems, there is a possibility that the gene of interest will become integrated into an inactive region of chromatin. Thus, scientists have to ensuring adequate and appropriate integration of a foreign gene (i.e. recombinant exogenous DNA) in the chromosome, which is labour-intensive and time-consuming.

At the same time, once the protein has been produced in the host cells, post-translational modification must ensure proper folding and/or glycosylation of protein of interest. For example, when unfolded proteins accumulate in the endoplasmic reticulum, the transcription of genes encoding chaperones and foldases are activated. However, misfolding of proteins can still occur and cause accumulation of intracellular aggregates (i.e. inclusion bodies) that can cause structural strains to the cells. The production of inactive proteins also represents an energetic drain and metabolic load. In addition, if the host cell is a bacterial cell, the protein produced may aggregate due to the lack of disulphide bond formation caused by the reducing environment of bacterial cytosol.

Other post-translational modification issues that one must consider include solubility (i.e. how easily can the protein be solubilized and renatured), proteolytic processing (i.e. signal peptides needed to direct proteins to various cellular compartments must be cleaved to obtain functional protein, low signal peptidase activity, which can limit the production of recombinant proteins), glycosylation capabilities of host cells (i.e. recombinant proteins may present macroheterogenous (differences in site occupancy) or microheterogenous (differences in the structures of oligosaccharides between glycosylation sites), factors that affecting glycosylation (for example, the synthesis of the dolicholphosphate oligosaccharide can limit the extent of glycosylation and artificially inducing such glycosylation in CHO cells have been shown to not work and the amount of sugar nucleotides and transport of sugar nucleotides to the endoplasmic reticulum or Golgi apparatus affect the rate of glycosylation), and other post-translational modifications factors (such as myristoylation, palmitoylation, isoprenylation, phosphorylation, sulfation, C-terminal amidation, β-hydroxylation, methylation, and the like).

Transport and localisation of proteins also pose multiple problems as location at which proteins are synthesized affects the purification process and the success of producing the correct protein. Location also depends on the characteristics of the protein where small proteins that are susceptible to proteolysis must be produced in inclusion bodies.

The use of animal cells also requires the person skilled in the art to consider cellular fragility and complex nutritional requirements of cells, need for growth factors and hormones (of the animal cells) to grow, possible contaminants of final products with virus and/or prions, difficulty in recovering extracellular proteins from serum-containing media, designing relevant gene transfer method based on the animal cell used, on whether the animal cells being able to cater to large scale protein production, and the like. Furthermore, a major drawback that emerges from altering the glycosylation machinery in vivo is the resulting heterogeneity of products, given the variety of pathways that can be followed. In spite of this, and given the subtle differences that exist between glycans obtained in commonly used mammalian cell lines and those associated with glycoproteins synthesized in human cells, cloning glycosyl-transferases into common mammalian cell lines has proved useful for the expression of humanized N-glycoproteins and O-glycoproteins.

One such specific example is the progress in the generation of retinal used for the treatment of macular degeneration of the eyes. Whilst there had been progress, it remains challenging to elucidate the underlying regulatory programs because differentiation protocols are laborious, variability in differentiation efficiency and often result in low retinal pigment epithelium (RPE) yields. Briefly, there are two different differentiation protocols for RPE has been employed thus far, spontaneous and stepwise directed differentiation. In the first method, cells are cultured in the absence of extrinsic growth factors and the RPE pigmented sheet was shown to be obtained after more than 180 days in culture. While, the second directed differentiation protocol involves extrinsic addition of transcription factors, and or using cocktail of numerous growth factors and or small molecules. Use of small molecules growth factors face significant practical challenges such as, possibility of off-target effects affecting interlaying signaling networks, delay in expression, expensive, timing of addition and purity due to batch-to-batch variability. In the case of direct reprogramming, a study reported the direct conversion of fibroblasts into RPE cells by expressing eight transcription factor coding genes (cMyc, Klf4, Nrl, Crx, Rax, Pax6, Mitf and Otx2) in combination with growth factors (Activin A/SHH) and small molecule (Retinoic acid). In line with this, another study identified and ectopically expressed a set of nine candidate transcription factor coding genes (PAX6, OTX2, LHX2, MITF, SIX3, SOX9, GLIS3, FOXD1 and ZNF92) using lentiviral based Tet-On expression system in human fetal fibroblast line to generate RPE lines. Some of the major drawbacks from these two studies is the need for activation of eight to nine genes, this might cause overloading in the cells due to the lentiviral integration of whole cDNAs and packaging of cDNAs in the lentivirus and efficient expression is also a critical challenge.

In view of the above, although there has been great advancements in generating specialized mature cells (such as RPE cells) there remains the need for a simple, cost-effective, robust differentiation protocol in order to reduce the heterogeneity and improve differentiated mature cell yields.

Accordingly, there is a need to provide an alternative method for altering a differentiation status of a cell.

### SUMMARY

In one aspect, there is provided method of altering a differentiation status of a cell, the method comprising: modulating the expression of one or more differentiation factors with a nuclease-deactivated Cas9 (dCas9) fusion protein, the dCas9 fusion protein comprising dCas9 and an effector comprising a transcriptional regulator, optionally the transcription regulator is a transcriptional activator.

In various embodiments, the method further comprising: providing a guide RNA (gRNA) in the cell, wherein the gRNA is capable of guiding the dCas9 fusion protein to a target site that is/that is in proximity of a promoter region of the one or more differentiation factors to allow the dCas9 fusion protein to modulate the expression of the one or more differentiation factors.

In various embodiments, the target site that is/that is in proximity of the promoter region is within an about −300 base pairs (bp) to about +5 bp window of the promoter region.

In various embodiments, the method further comprising: providing an activator module comprising a RNA-binding protein capable of binding to the gRNA, optionally wherein the RNA-binding protein comprises MS2 coat protein (MCP).

In various embodiments, the activator module further comprises one or more transcriptional activators, optionally the transcriptional activator is selected from the group consisting of VP64, p65, HSF1, Rta and combinations thereof. In various embodiments, the activator module comprises p65 and/or HSF1.

In various embodiments, the dCas9 fusion protein comprises VP64 and optionally, p65 and/or Rta.

In various embodiments, the method further comprising expressing the dCas9 fusion protein, optionally a dCas9-VP64 fusion protein and/or a dCas9-VP64-p65-Rta (dCas9-VPR) fusion protein, prior to the modulating step.

In various embodiments, the method comprises modulating the expression of one or more differentiation factors with a CRISPR/dCas9 synergistic activation mediators (CRISPR/dCas9-SAM) complex/dCas9 ribonucleoprotein complex (e.g. a complex comprising the dCas9 fusion protein)/dCas9-VP64/dCas9-VPR/dCas9-VP64 and MS2-P65-HSF1.

In various embodiments, the one or more differentiation factors comprises transcription factors.

In various embodiments, the cell is a stem cell, stem cell-like cell, a progenitor cell or a precursor cell, optionally the cell comprises one that is selected from the group consisting of: embryonic stem cell (e.g. hESC3), adult stem cell, induced pluripotent stem cell (iPSC), mesenchymal stem cell (MSC), human embryonic kidney cell (HEK293) and the like.

In various embodiments, the method is a method of differentiating a cell.

In various embodiments, the one or more differentiation factors influence an expression of a neuroprogenitor gene and/or a retinal pigment epithelium (RPE)-associated gene, optionally the RPE-associated gene comprises a gene associated with a mature RPE/RPE specific mature gene, a gene associated with pigmentation/RPE specific pigmentation gene or early eye field gene.

In various embodiments, the one or more differentiation factors is selected from the group consisting of PAX6, MITF, OTX2 and combinations thereof.

In various embodiments, the one or more differentiation factors is selected from the group consisting of LHX2, RAX2, Tyrosinase, CRALBP, BEST1, RPE65, PEDF, pmel17, PYR, Tryp1, Tryp2, CRX and combinations thereof.

In various embodiments, the cell produced from the method expresses premelanosome marker 17 (PMEL17), optionally the expression of PMEL17 in the produced cell is at least about 50%.

In various embodiments, the cell produced from the method expresses Pax6, optionally the cell is a neuroprogenitor cell.

In various embodiments, the method is a method of maintaining and/or expanding a cell, optionally maintaining and/or expanding a haematopoietic stem cells.

In various embodiments, the one or more differentiation factors is selected from the group consisting of erythropoietin (EPO), stem cell factor (SCF), thrombopoietin (TPO), granulocyte-macrophage colony-stimulating factor (GM-CSF), granulocyte-colony stimulating factor (G-CSF), and combinations thereof.

In various embodiments, the method is free of modulating the expression of a transcription activator selected from the group consisting of: cMyc, Klf4, Nrl, Crx, Rax, LHX2, SIX3, SOX9, GLIS3, FOXD1, ZNF92 , C11orf9 and combinations thereof directly via the dCas9 fusion protein.

In various embodiments, the method is free of the use of a gRNA specific to a target site that is/that is in proximity of a promoter region of: cMyc, Klf4, Nrl, Crx, Rax, LHX2, SIX3, SOX9, GLIS3, FOXD1, ZNF92, C11orf9 and combinations thereof.

In various embodiments, the method is free of exogenous growth factor, free of inducible system, and/or is free of whole exogenous nucleic acid.

In various embodiments, modulating the expression of one or more differentiation factors comprises an endogenous activation of the one or more differentiation factors. In another aspect, there is provided a cell comprising a dCas9 fusion protein that is configured to modulate the expression of one or more differentiation factors, the dCas9 fusion protein comprising dCas9 and an effector, or progenies thereof.

In various embodiments, the cell comprises a guide RNA (gRNA) capable of guiding the dCas9 fusion protein to a target site that is/that is in proximity of the promoter region of the one or more differentiation factors to allow the dCas9 fusion protein to modulate the expression of the one or more differentiation factors.

In yet another aspect, there is provided a cell having a second differentiation status (or its progenies thereof) that was differentiated from a cell having a first differentiation status, wherein the cell having the first differentiation status comprises a dCas9 fusion protein that is configured to modulate the expression of one or more differentiation factors, the dCas9 fusion protein comprising dCas9 and an effector.

In various embodiments, the cell having the second differentiation status is devoid of a dCas9 fusion protein or a CRISPR/dCas9-SAM complex.

In yet another aspect, there is provided a guide RNA (gRNA) to a target site that is or that is in proximity of the promoter region of one or more differentiation factors to modulate the expression of the one or more differentiation factors, wherein the gRNA is configured to guide a fusion protein selected from the group consisting of dCas9 fusion protein, CRISPR/dCas9 synergistic activation mediators (CRISPR/dCas9-SAM) complex, dCas9 ribonucleoprotein complex, dCas9-VP64, dCas9-VPR, dCas9-VP64, and MS2-P65-HSF1.

In various embodiments, at least a portion of the guide RNA is capable of binding to the target site/target genomic locus that is in an about −300 base pairs (bp) to about +5 bp window of the promoter region of one or more differentiation factors selected from the group consisting of PAX6, MITF, OTX2, EPO, SCF, TPO, GM-CSF, G-CSF, and combinations thereof.

In various embodiments, the gRNA has at least about 80% identity with a sequence selected the group consisting of SEQ ID NO: 1 (AATGTGTGTGTGCCGGCGCC), SEQ ID NO: 2 (GCCAGCACACCTATGCTGAT), SEQ ID NO: 3 (GCTTCGCTAATGGGCCAGTG), SEQ ID NO: 4 (ACAATAAAATGGGCTGTCAG), SEQ ID NO: 5 (GAGTGAGAGATAAAGAGTGT), SEQ ID NO: 6 (CGGGCCGAACTACAGATCCC), SEQ ID NO: 7 (CCAAACAGGAGTTGCACTAG), SEQ ID NO: 8 (AGCTGTAGTTTTCGTGGGAG), SEQ ID NO: 9 (GCGGGGGAGAGGCAACGTGG), SEQ ID NO: 10 (CTGTACCCTTGAAGCAAGTG), SEQ ID NO: 11 (GAACATTCTGGTAATGTCGG), SEQ ID NO: 12 (GCGTCAAAAAGTTGCCAGAG), SEQ ID NO: 13 (AACAGGCCGCTGCTGCACGG), SEQ ID NO: 14 (GATTGACACATCTAAGCCAG), SEQ ID NO: 15 (TAAAAACACACAACAGGGGG), SEQ ID NO: 76 (GGGGTGGCCCAGGGACTCTG), SEQ ID NO: 77 (TGTGCGTGAGGGGTCGCCAG), SEQ ID NO: 78 (GCCCCTGCTCTGACCCCGGG), SEQ ID NO: 79 (GGAGAGGCTGTGTGCGTGAG), SEQ ID NO: 80 (GAACTGTATAAAAGCGCCGG), SEQ ID NO: 81 (CCTAATCTGCCAAACTTCTG), SEQ ID NO: 82 (GAGGCGTGTCCGGAGCAGGC), SEQ ID NO: 83 (GGTAGGCGAGAAGCAGGCAA), SEQ ID NO: 84 (TCCTTCCCTTCCGGAGCCCG), SEQ ID NO: 85 (GAGCCACCAGACACTGGTGA), SEQ ID NO: 86 (CCCTATCCAAATCTTCTCCG), SEQ ID NO: 87 (ACTTCTGCCCAATCAGAGAA), SEQ ID NO: 88 (AAGAGAAGGCGTCACTTCCG), SEQ ID NO: 89 (AGCAGGTCATACGCCTGCCT), SEQ ID NO: 90 (AAGAGCTCTTAAATACACAG), SEQ ID NO: 91 (GTGACCACAAAATGCCAGGG), SEQ ID NO: 92 (CGGGGGAACTACCTGAACTG), SEQ ID NO: 93 (GGCCCTTATCAGCCACACAT), SEQ ID NO: 94 (AGGCTCACCGTTCCCATGTG), SEQ ID NO: 95 (GTGTCCAAGACAATGCAGGG), SEQ ID NO: 96 (GGGCAAGGCGACGTCAAAGG), SEQ ID NO: 97 (GCGAAAGTTTTGTGAAATTG), SEQ ID NO: 98 (GGGGGGCAAGGCGACGTCAA), and SEQ ID NO: 99 (CACCAAATTTGCATAAATCC).

In various embodiments, the gRNA has about 15 bp to about 25 bp.

In various embodiments, the gRNA is a single/short gRNA (sgRNA).

In yet another aspect, there is provided a set of gRNA comprising at least two of the gRNA of any of claims 25 to 29, wherein the gRNA is selected from the group consisting of: a gRNA that is specific to a target site that is/that is in proximity of the promoter region of PAX6, a gRNA that is specific to a target site that is/that is in proximity of the promoter region of MITF and a gRNA that is specific to a target site that is/that is in proximity of the promoter region of OTX2.

In yet another aspect, there is provided an oligonucleotide/primer for cloning a gRNA as described herein, the oligonucleotide/primer having at least about 80% with a sequence selected from Table 2 below:

TABLE 2

| | | SEQ ID |
|---|---|---|
| Name | Sequence | NO. |
| Pax6_1_Fwd | CACCGACAATAAAATGGGCTGTCAG | 16 |
| Pax6_1_Rev | AAACCTGACAGCCCATTTTATTGTC | 17 |
| Pax6_2_Fwd | CACCGGAGTGAGAGATAAAGAGTGT | 18 |
| Pax6_2_Rev | AAACACACTCTTTATCTCTCACTCC | 19 |
| Pax6_3_Fwd | CACCGGCCAGCACACCTATGCTGAT | 20 |
| Pax6_3_Rev | AAACATCAGCATAGGTGTGCTGGCC | 21 |
| Pax6_4_Fwd | CACCGAATGTGTGTGTGCCGGCGCC | 22 |
| Pax6_4_Rev | AAACGGCGCCGGCACACACACATTC | 23 |
| Pax6_5_Fwd | CACCGGCTTCGCTAATGGGCCAGTG | 24 |
| Pax6_5_Rev | AAACCACTGGCCCATTAGCGAAGCC | 25 |
| MITF_1_Fwd | CACCGCGGGCCGAACTACAGATCCC | 26 |
| MITF_1_Rev | AAACGGGATCTGTAGTTCGGCCCGC | 27 |
| MITF_2_Fwd | CACCGCCAAACAGGAGTTGCACTAG | 28 |
| MITF_2_Rev | AAACCTAGTGCAACTCCTGTTTGGC | 29 |
| MITF_3_Fwd | CACCGGCGGGGGAGAGGCAACGTGG | 30 |
| MITF_3_Rev | AAACCCACGTTGCCTCTCCCCCGCC | 31 |
| MITF_4_Fwd | CACCGAGCTGTAGTTTTCGTGGGAG | 32 |
| MITF_4_Rev | AAACCTCCCACGAAAACTACAGCTC | 33 |
| MITF_5_Fwd | CACCGCTGTACCCTTGAAGCAAGTG | 34 |
| MITF_5_Rev | AAACCACTTGCTTCAAGGGTACAGC | 35 |
| OTX2_1_Fwd | CACCGGCGTCAAAAAGTTGCCAGAG | 36 |
| OTX2_1_Rev | AAACCTCTGGCAACTTTTTGACGCC | 37 |
| OTX2_2_Fwd | CACCGGAACATTCTGGTAATGTCGG | 38 |
| OTX2_2_Rev | AAACCCGACATTACCAGAATGTTCC | 39 |
| OTX2_3_Fwd | CACCGTAAAAACACACAACAGGGGG | 40 |
| OTX2_3_Rev | AAACCCCCCTGTTGTGTGTTTTTAC | 41 |
| OTX2_4_Fwd | CACCGAACAGGCCGCTGCTGCACGG | 42 |
| OTX2_4_Rev | AAACCCGTGCAGCAGCGGCCTGTTC | 43 |
| OTX2_5_Fwd | CACCGGATTGACACATCTAAGCCAG | 44 |
| OTX2_5_Rev | AAACCTGGCTTAGATGTGTCAATCC | 45 |

In yet another aspect, there is provided a composition comprising: a dCas9 fusion protein, the dCas9 fusion protein comprising dCas9 and an effector; a gRNA, optionally a sgRNA, wherein the gRNA is capable of guiding the dCas9 fusion protein to a target site that is/that is in proximity of the promoter region of one or more differentiation factors to allow the dCas9 fusion protein to modulate the expression of the one or more differentiation factors; and optionally an activator module comprising a RNA-binding protein capable of binding to the gRNA, further optionally wherein the RNA-binding protein comprises MS2 coat protein (MCP).

In yet another aspect, there is provided a kit comprising reagents for altering a differentiation status of a cell, the kit comprising: a nucleic acid transcribing a gRNA, optionally a sgRNA, that is capable of guiding a dCas9 fusion protein to a target site that is/that is in proximity of the promoter region of the one or more differentiation factors to allow the dCas9 fusion protein to modulate the expression of the one or more differentiation factors.

In various embodiments, the kit further comprising one or more of the following:
a) a second or further nucleic acid transcribing a second or further gRNA, optionally a sgRNA, that is capable of guiding a dCas9 fusion protein to a target site that is/that is in proximity of the promoter region of the one or more differentiation factors to allow the dCas9 fusion protein to modulate the expression of the one or more differentiation factors;
b) a nucleic acid encoding a dCas9 fusion protein, optionally a dCas9-VP64 fusion protein and/or a dCas9-VPR fusion protein;
c) a nucleic acid encoding an activator module comprising a RNA-binding protein capable of binding to the gRNA, optionally wherein the RNA-binding protein comprises MS2 coat protein (MCP);
d) a fusion protein selected from the group consisting of CRISPR/dCas9 synergistic activation mediators (CRISPR/dCas9-SAM) complex, dCas9 ribonucleoprotein complex, dCas9-VP64, dCas9-VPR, dCas9-VP64, and MS2-P65-HSF1;
e) one or more oligonucleotide/primer having at least about 80% identity with a sequence selected from Table 2;
f) a viral vector, a virus packaging plasmid and/or a virus expression vector;
g) one or more probes, capture agents, dyes, labels, nucleotides, salts, buffering agents, various additives, PCR enhancers and combinations thereof; and
h) instructions for use.

In yet another aspect, there is provided a method of treating a disease, the method comprising transplanting the cell as described herein to a patient in need thereof.

In various embodiment, the disease is an eye disease/disorder, optionally wherein the eye disease/disorder is selected from the group consisting of macular degeneration, acute macular degeneration (AMD), atrophic age-related macular degeneration (atrophic AMD), dry age-related macular degeneration (Dry-type AMD), retinitis pigmentosa (RP), Stargardt's disease, and myopia.

### DESCRIPTION OF EMBODIMENTS

This disclosure describes a method for the differentiation of pluripotent stem cells into specialized cells. Whilst not wishing to be bound by theory, but merely to provide an example, the inventors tested the hypothesis of the method of altering cells as described herein by generating neuroprogenitor cells and/or mature retinal pigment epithelium (RPE) cells, maintaining and/or expanding haematopoietic cells, and the like. In particular, the method as described herein was shown to be able to generate mature RPE cells by endogenous activation of only three transcription factors (PAX6, MITF and OTX2) using CRISPR/dCas9-SAM. Pigmented, cobblestone morphology of highly pure RPE cell cultures based on the expression level of premelanosome marker 17 (PMEL17) were shown to be generated within only 40 days of activation of transcription factors in RPE maintenance media (RPEM). The technology surprisingly has advantages such as: minimal set of transcription factor required for efficient differentiation, cost-effective (doesn't require any growth factors and/or small molecules), endogeneous activation of genes without the need to extrinsically add the whole cDNA and can obtain pigmented foci, visible to the naked eye rapidly within 40 days of gene activation.

In some examples, this disclosure describes a method of using CRISPR/dCas9 synergistic activation mediators (SAM) based targeted activation of transcription factors required for rapid and cost-efficient differentiation of human pluripotent stem cells to functional retinal pigment epithelium (RPE) cells. Disclosed herein is a simple differentiation method that is exemplified by the activation of only three key transcription factors such as PAX6, MITF and OTX2 in iPSC line generated from IMR90 fetal lung fibroblasts. Thus, disclosed herein is an example of a general method of differentiating pluripotent stem cells by CRISPR/dCas9 mediated endogenous gene activation to other lineages that are difficult to make such as retina, hair, muscle, blood and pancreatic islets. In particular, the present disclosure relates to a method for the differentiation of pluripotent stem cells by endogenous activation of transcription factors.

Accordingly, the present disclosure provides a method of altering a differentiation status of a cell, the method comprising: modulating the expression of one or more differentiation factors with a nuclease-deactivated Cas9 (dCas9) fusion protein, the dCas9 fusion protein comprising dCas9 and an effector comprising a transcriptional regulator; and optionally culturing/growing the cell under conditions that support the altered differentiation status. In some examples, the effector comprises a transcriptional activator.

In one aspect, there is provided a method of altering a differentiation status of a cell, the method comprising: modulating the expression of one or more differentiation factors with a nuclease-deactivated Cas9 (dCas9) fusion protein, the dCas9 fusion protein comprising dCas9 and an effector comprising a transcriptional regulator, optionally the transcription regulator is a transcriptional activator.

As used herein, the term “differentiation” refers to the process of a cell from being less specialized (or de-differentiated, or undifferentiated, or less differentiated) to develop into more specialized cells of the same or different cell type to the original target cell. In some examples, the one or more differentiation factors, when activated/upregulated/over-expressed, promote cell differentiation. As used herein, differentiation factors may include, but is not limited to transcription factors and non-transcription factors and their associated genes. As such, in various embodiments, the one or more differentiation factors comprises transcription factors.

As the method of the present disclosure is capable of altering the differentiation status of a cell, in various embodiments, the method is a method of differentiating a cell.

In some examples, the method further comprising: introducing/expressing/providing a guide RNA (gRNA), optionally a single/short guide RNA (sgRNA), in the cell, wherein the gRNA is capable of guiding the dCas9 fusion protein to a target site that is the promoter region/that is in proximity of the promoter region, optionally a target site that is within 200 base pairs upstream of the promoter region, of the one or more differentiation factors to allow the dCas9 fusion protein to modulate the expression of the one or more differentiation factors.

Without wishing to be bound by theory, gRNA targeting of other exon sites is considered and is believed to advantageously allow an increase expression due to splicing of the exons together. Therefore, in some examples, the method further comprising: introducing/expressing/providing a guide RNA (gRNA), optionally a single/short guide RNA (sgRNA), in the cell, wherein the gRNA is capable of guiding the dCas9 fusion protein to a target site that is on other exon sites (or on another exon site) from the one or more differentiation factors. In some examples, the target site may be on other exon sites or another exon site that is further from the promoter region of the one or more differentiation factors.

In some examples, method may comprise introducing/expressing/providing a plurality of gRNAs in the cell, the plurality of gRNAs being specific to different target sites. In various embodiments, the amount of each gRNA in the plurality of gRNAs expressed/introduced in the cell is substantially the same, further optionally wherein the method comprises introducing a single vector encoding the plurality of gRNAs (e.g. three gRNAs) into the cell e.g. to obtain a uniform expression of the plurality of gRNAs in the cell/cell population. As used herein, the term “target” refers to the site of interest or test site that may be used interchangeably and refers to the region of the target gene, which is targeted by the CRISPR/dCas9-based system (which may be without the PAM). In various embodiments, CRISPR/Cas9-based system may include at least one gRNA, wherein the gRNAs target different DNA sequences on the target gene. The target DNA sequences may be overlapping. The target sequences or protospacer is followed by a PAM sequence at the 3′ end of the protospacer.

In various embodiments, the method further comprising: providing a guide RNA (gRNA) in the cell, wherein the gRNA is capable of guiding the dCas9 fusion protein to a target site that is/that is in proximity of a promoter region of the one or more differentiation factors to allow the dCas9 fusion protein to modulate the expression of the one or more differentiation factors.

In some examples, the gRNA comprises the target site that is/that is in proximity of the promoter region is within an about −300 base pairs (bp) to about +5 bp window, an about −250 bp to about +3 bp window or an about −200 bp to about +1 bp window of the promoter region. In various embodiments, the target site that is/that is in proximity of the promoter region is within an about −300 base pairs (bp) to about +5 bp window of the promoter region.

In various embodiments, the method further comprising: providing a guide RNA (gRNA) in the cell, wherein the gRNA is capable of guiding the dCas9 fusion protein to a target site that is/that is on other exon or in another exon of the one or more differentiation factors (or a promoter site of one or more differentiation factors) to allow the dCas9 fusion protein to modulate the expression of the one or more differentiation factors. As such, in some examples, the gRNA comprises the target site that is/that is in another exon is more than about −300 base pairs (bp), more than about −400 bp, more than about −500 bp or more than −1000 bp, or more.

In some examples, the gRNA comprises a stem-loop/hairpin structure, optionally a MS2 stem-loop/hairpin structure. In some examples, the method may further comprise introducing/expressing an activator module comprising a RNA-binding protein capable of binding to the stem-loop/hairpin structure of the gRNA, optionally wherein the RNA-binding protein comprises MS2 coat protein (MCP). In various embodiment, the method further comprises: providing an activator module comprising a RNA-binding protein capable of binding to the gRNA, optionally wherein the RNA-binding protein comprises MS2 coat protein (MCP). In some examples, gRNAs of the present disclosure do not exist in nature or is not a naturally occurring nucleic acid.

In various embodiments, the activator module further comprises one or more transcriptional activators, optionally the transcriptional activator is selected from the group consisting of VP64, p65, HSF1, Rta and combinations thereof. In various embodiments, the activator module comprises p65 and/or HSF1. In various embodiments, the dCas9 fusion protein comprises VP64 and optionally, p65 and/or Rta. As such, in various embodiments, the method further comprising expressing the dCas9 fusion protein, optionally a dCas9-VP64 fusion protein and/or a dCas9-VP64-p65-Rta (dCas9-VPR) fusion protein, prior to the modulating step.

In various examples, the method may further comprise: introducing the dCas9 fusion protein (optionally a dCas9-VP64 fusion protein and/or a dCas9-VPR fusion protein) and/or a nucleic acid encoding the same into a cell, optionally via one or more of the following methods: viral vector-mediated delivery, extracellular vesicle-mediated delivery including exosome-mediated delivery, electroporation, delivery by lipid-based carrier (e.g. lipofectamine, lipid nanoparticle etc.), delivery by polymeric carrier (e.g. polymeric nanoparticle), complexation with nanoparticle (e.g. gold nanoparticle), conjugation with cell-penetrating peptide (CPP) (e.g. a CPP containing a nuclear localization sequence), in vitro complexed RNPs (ribonucleoprotein) delivery (see for example https://blog.addgene.org/crispr-101-ribonucleoprotein-rnp-delivery) e.g. for transient activation and delivery as RNAs of the dCas9 fusion protein (optionally together with an activator module and/or sgRNAs) e.g. for transient expression, prior to the modulating step.

In some examples, the introducing step may comprise transducing a cell with a viral vector (or a supernatant comprising the viral vector) containing the nucleic acid encoding the dCas9 fusion protein, optionally a dCas9-VP64 fusion protein and/or a dCas9-VPR fusion protein, and optionally subjecting the cell to antibiotic selection (e.g. hygromycin B or blasticidin etc.).

In some examples, the method may further comprise: introducing the gRNA or a nucleic acid transcribing the same into the cell optionally via one or more of the following methods: viral vector-mediated delivery, extracellular vesicle-mediated delivery including exosome-mediated delivery, electroporation, delivery by lipid-based carrier (e.g. lipofectamine, lipid nanoparticle etc.), delivery by polymeric carrier (e.g. polymeric nanoparticle), complexation with nanoparticle (e.g. gold nanoparticle), conjugation with cell-penetrating peptide (CPP) (e.g. a CPP containing a nuclear localization sequence), in vitro complexed RNPs (ribonucleoprotein) delivery (see for example https://blog.addgene.org/crispr-101-ribonucleoprotein-rnp-delivery) e.g. for transient activation, nucleofection/electroporation of in vitro synthesized sgRNA e.g. into stable dCas9-VP64/dCas9-VPR fusion protein and/or MS2-p65-HSF1 expressing cell lines e.g. to generate transient activation and delivery, e.g. direct delivery, of the sgRNA (optionally together with a dCas9 fusion protein and/or an activator module) e.g. for transient expression.

In some examples, the introducing step comprises transducing the cell with a viral vector (or a supernatant comprising the viral vector) containing the nucleic acid transcribing the gRNA, and optionally subjecting the cell to antibiotic selection (e.g. hygromycin B or blasticidin etc.).

In some examples, the method may further comprise: introducing the activator module (optionally MCP-p65-HSF1) or a nucleic acid encoding the same into the cell, optionally via one or more of the following methods: viral vector-mediated delivery, extracellular vesicle-mediated delivery including exosome-mediated delivery, electroporation, delivery by lipid-based carrier (e.g. lipofectamine, lipid nanoparticle etc.), delivery by polymeric carrier (e.g. polymeric nanoparticle), complexation with nanoparticle (e.g. gold nanoparticle), conjugation with cell-penetrating peptide (CPP) (e.g. a CPP containing a nuclear localization sequence), in vitro complexed RNPs (ribonucleoprotein) delivery (see for example https://blog.addgene.org/crispr-101-ribonucleoprotein-rnp-delivery) e.g. for transient activation and delivery as RNAs of the activator module (optionally together with dCas9 fusion protein and/or sgRNAs) e.g. for transient expression. In some examples, the introducing step may comprises transducing the cell with a viral vector (or a supernatant comprising the viral vector) containing the nucleic acid encoding the activator module, and optionally subjecting the cell to antibiotic selection (e.g. hygromycin B or blasticidin etc.).

In some examples, the method may further comprise: transfecting a host cell, e.g. a HEK293T cell, in a medium with a virus packaging plasmid, an envelope plasmid, a virus expression vector and/or a nucleic acid encoding for a protein/RNA selected from the group consisting of: the dCas9 fusion protein (optionally a dCas9-VP64 fusion protein and/or a dCas9-VPR fusion protein), the gRNA, the activator module (optionally MCP-p65-HSF1) and combinations thereof; collecting/harvesting the supernatant and optionally purifying/concentrating the supernatant, thereby obtaining the viral vector contained in the supernatant.

In some examples, the viral vector may comprise an integrating viral vector or a non-integrating viral vector. In some examples, the viral vector may be selected from the group consisting of lentivirus, adenovirus, retrovirus, and adeno-associated virus (AAV), and chimeric synthetic viral vector (e.g. a viral vector containing unique features of each of the (various) natural virus vectors) optionally wherein the viral vector comprises lentivirus.

In some examples, the method comprises modulating the expression of one or more differentiation factors with a CRISPR/dCas9 synergistic activation mediators (CRISPR/dCas9-SAM) complex/dCas9 ribonucleoprotein complex (e.g. a complex comprising the dCas9 fusion protein).

The term “Cas” or “CRISPR-associated (cas)” refers to genes often associated with CRISPR repeated-spacer arrays. As such, “Cas9” refers to a nuclease from type II CRISPR systems, an enzyme specialized for generating double-strand breaks in DNA, with two active cutting sites, one for each strand of the double helix. tracrRNA and spacer RNA may be combined into a single-guide RNA” (sgRNA) molecule that mixed with Cas9 could find and cleave DNA targets through Waston-Crick pairing between the guide sequence within the sgRNA and the target DNA sequence. In some examples, the method may comprise providing a cell/cell population that transiently or stably expresses the dCas9 fusion protein, the gRNA (such as sgRNA), the activator module and/or the CRISPR/dCas9-SAM complex, optionally wherein the method comprises providing a cell/cell population that transiently or stably expresses sgRNA. In some examples, dCas9 is/is derived from/is modified from a Cas9 protein selected from the group consisting of: Streptococcus pyogenes Cas9, Streptococcus aureus Cas9, Campylobacter jejuni Cas9, Neisseria meningitidis (NM) Cas9, Streptococcus thermophilus (ST) Cas9, Treponema denticola (TD) Cas9, and Francisella novicida Cas9.

In some examples, the method is free of (or does not comprise) expressing a catalytically active Cas9 nuclease. In some examples, the method does not comprise (or free of) cleaving a genome/nucleic acid with Cas9 nuclease e.g. to integrate a gene/transcription factor.

In some examples, the one or more differentiation factors may influence cell differentiation, cell dedifferentiation, cell reprogramming (e.g. from somatic cell) or cell transdifferentiation. In some examples, the method may comprise modulating the expression of one or more differentiation factors comprises activating/promoting/enhancing/increasing/upregulating the expression of one or more differentiation factors. In various embodiments, the method comprises modulating the expression of one or more differentiation factors with a CRISPR/dCas9 synergistic activation mediators (CRISPR/dCas9-SAM) complex/dCas9 ribonucleoprotein complex (e.g. a complex comprising the dCas9 fusion protein)/dCas9-VP64/dCas9-VPR/dCas9-VP64 and MS2-P65-HSF1.

Without wishing to be bound by theory, it is believed that the method as described herein may be applied to many different cell types, including, but not limited to retinal pigment epithelium (RPE), stem cells (for example MSC sources), pluripotent stem cells (such as hESC and hiPSC), and other cell lineages such as CD34+ cells or erythroblasts, and cells from other species. In some examples, the cell may be a stem cell, stem cell-like cell, a progenitor cell or a precursor cell. In some examples, the cell comprises a totipotent stem cell, a pluripotent stem cell or a multipotent stem cell. In some examples, the cell may be one a cell such as, but is not limited to, embryonic stem cell (e.g. hESC3), adult stem cell, induced pluripotent stem cell (iPSC), mesenchymal stem cell (MSC), human embryonic kidney cell (HEK293) and the like. In various embodiments, the cell is a stem cell, stem cell-like cell, a progenitor cell or a precursor cell, optionally the cell comprises one that is selected from the group consisting of: embryonic stem cell (e.g. hESC3), adult stem cell, induced pluripotent stem cell (iPSC), mesenchymal stem cell (MSC), human embryonic kidney cell (HEK293) and the like. In various embodiments, the cell may not comprise fibroblast such as (human) fetal fibroblast or (human) foreskin fibroblast. In various embodiments, the cell may be an animal cell (e.g. bovine cell, fish cell, chicken cell including chicken embryonic fibroblast etc.), optionally a mammalian cell, or a human cell.

In various embodiments, the method of the present disclosure may be a method of producing/engineering a specialized cell, optionally a human specialized cell, such as, but is not limited to retina cell, hair cell, blood cell, CD34, erythroblast, retinal pigment epithelium (RPE), pancreatic islet cell, muscle cell, and the like.

In various embodiments, the method may be a method of producing/engineering a (human) RPE cell/RPE cell line/RPE cell population/RPE sheet, optionally a mature (human) RPE cell/RPE cell line/RPE cell population/RPE sheet.

In various embodiments, the one or more differentiation factors (or transcription factors) influence an expression of a retinal pigment epithelium (RPE)-associated gene and/or a neuroprogenitor gene. In various embodiments, the retinal pigmented epithelium (RPE)-associated gene comprises a gene associated with a mature RPE/RPE specific mature gene, a gene associated with pigmentation/RPE specific pigmentation gene or early eye field gene. In various embodiments, the neuroprogenitor gene may comprise one or more gene associated with rods and/or cones cells.

In various embodiments, the one or more differentiation factors is selected from the group consisting of PAX6, MITF, OTX2 and combinations thereof. In various embodiments, the one or more differentiation factors is selected from the group consisting of LHX2, RAX2, Tyrosinase, CRALBP, BEST1, RPE65, PEDF, pmel17, PYR, Tryp1, Tryp2, CRX and combinations thereof.

In various examples, the method further comprises culturing/growing the cell under conditions that support neuroprogenitor differentiation to obtain an intermediate neuroprogenitor cell/neuroprogenitor cell/neuroprogenitor cell line, such as but is not limited to rods cells, cones cells, and the like. In various examples, the culturing/growing the cell under conditions that support neuroprogenitor differentiation comprises culturing/growing the cell in one or more of the following: a neuroprogenitor maintenance media, matrigel and Laminin 521 matrix coating. In some examples, the produced/engineered intermediate neuroprogenitor cell/neuroprogenitor cell/neuroprogenitor cell line expresses PAX6. In some examples, the method is capable of producing high yields of a neuroprogenitor cell/neuroprogenitor cell/neuroprogenitor cell line. In some examples, the expression of PAX6 in produced/engineered neuroprogenitor cell/neuroprogenitor cell/neuroprogenitor cell line is at least about 50%, at least about 51%, at least about 52%, at least about 53%, at least about 54%, at least about 55%, at least about 55%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97% or at least about 98% (e.g. at any day between Day 1 to Day 18 post-transfection/transduction).

In various examples, the method further comprises culturing/growing the cell under conditions that support RPE differentiation to obtain a mature human RPE cell/RPE cell line/RPE cell population/RPE sheet. In various examples, the culturing/growing the cell under conditions that support RPE differentiation comprises culturing/growing the cell in one or more of the following: a RPE maintenance media, matrigel and Laminin 521 matrix coating. In some examples, the produced/engineered RPE cell/RPE cell line/RPE cell population/RPE sheet expresses premelanosome marker 17 (PMEL17). In some examples, the method is capable of producing high yields of a RPE cell/RPE cell population/RPE sheet. In some examples, the expression of PMEL17 in produced/engineered RPE cell population/RPE sheet is at least about 50%, at least about 51%, at least about 52%, at least about 53%, at least about 54%, at least about 55%, at least about 55%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97% or at least about 98% (e.g. at Day 18, 28 or 40 post transfection/transduction).

In various embodiments, the cell produced from the method expresses premelanosome marker 17 (PMEL17), optionally the expression of PMEL17 in the produced cell is at least about 50%. In some examples, the method of the present disclosure is capable of producing a highly pure RPE cell culture/population. For example, the cell may have more than (>) 90% PMEL17 or (>) 96% PMEL17.

In various examples, the expression of PAX6 in produced/engineered RPE cell population/RPE sheet is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97% or at least about 98% (e.g. at Day 18, 28 or 40 post transfection/transduction). In various embodiments, as shown in the Example section of the present disclosure, the produced/engineered RPE cell/RPE cell line/RPE cell population/RPE sheet has a pigmented, cobblestone morphology. As such, the produced/engineered RPE cell/RPE cell line/RPE cell population/RPE sheet is substantially similar (but not necessarily identical) in characteristics (including functional, behavioral characteristics etc.) to a naturally occurring RPE cell/RPE cell population/RPE sheet. In some examples, the produced/engineered RPE cell/RPE cell line/RPE cell population/RPE sheet comprises a pigmented foci, optionally wherein the pigmented foci is visible to the naked eye.

In some examples, the method is capable of producing a human RPE cell/RPE cell line/RPE cell population/RPE sheet, e.g. a mature human RPE cell/RPE cell line/RPE cell population/RPE sheet that comprises a pigmented foci that is visible to the naked eye, in no more than about 180 days, in no more than about 150 days, in no more than about 100 days, in no more than about 75 days, in no more than about 50 days, no more than about 49 days, no more than about 48 days, no more than about 47 days, no more than about 46 days, no more than about 45 days, no more than about 44 days, no more than about 43 days, no more than about 42 days, no more than about 41 days, no more than about 40 days, no more than about 39 days, or no more than about 38 days from the expressing step.

In some examples, the method comprises modulating the expression of no more than seven, no more than six, no more than about five, no more than about four, or no more than about three genes/transcription regulators/transcription activators directly via the dCas9 fusion protein. In some examples, the method comprises modulating the expression of no more than seven, no more than six, no more than about five, no more than about four, or no more than about three genes/transcription regulators/transcription activators directly via the dCas9 fusion protein, further wherein the method comprises modulating the expression of a gene/transcription regulator/transcription activator selected from the group consisting of PAX6, MITF, OTX2 and combinations thereof.

In various embodiments, the method as disclosed herein may produce intermediate neuroprogenitor cells that are characterized by the expression of Pax6. Thus, in various embodiments, the cell produced from the method expresses Pax6, optionally the cell is a neuroprogenitor cell. In various embodiments, the cell may be an intermediate neuroprogenitor cells that are expandable and can further differentiate into other lineages such as, but is not limited to, rod and/or cone cell types. In various embodiments, the neuroprogenitor cell may be characterized by the expression of PAX6 .

In various embodiments, the method as described herein is a method of maintaining and/or expanding a cell. In various embodiments, the method as described herein may be a method of maintaining and/or expanding a haematopoietic stem cell.

In various embodiment, wherein the method is a method of maintaining and/or expanding a cell (such as a haematopoietic stem cell), the one or more differentiation factors may include, but is not limited to, erythropoietin (EPO), stem cell factor (SCF), thrombopoietin (TPO), granulocyte-macrophage colony-stimulating factor (GM-CSF), granulocyte-colony stimulating factor (G-CSF), and combinations thereof.

In various examples, the expression of the one or more genes may include but is not limited to, erythropoietin (EPO), stem cell factor (SCF), thrombopoietin (TPO), granulocyte-macrophage colony-stimulating factor (GM-CSF), granulocyte-colony stimulating factor (G-CSF) in maintained and/or expanded cell (such as a haematopoietic stem cell) is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97% or at least about 98% (e.g. at day 3 or 4 post-transfection).

In some examples, the method does not comprise/is devoid of modulating the expression of a gene/transcription regulator/transcription activator selected from the group consisting of: cMyc, Klf4, Nrl, Crx, Rax, LHX2, SIX3, SOX9, GLIS3, FOXD1, ZNF92, C11orf9 and combinations thereof directly via the dCas9 fusion protein. In some examples, the method does not comprise/is devoid of the use of a gRNA specific to a target site that is/that is in proximity of a promoter region of: cMyc, Klf4, Nrl, Crx, Rax, LHX2, SIX3, SOX9, GLIS3, FOXD1, ZNF92, C11orf9 and combinations thereof. In various embodiments, the method is free of modulating the expression of a transcription activator selected from the group consisting of: cMyc, Klf4, Nrl, Crx, Rax, LHX2, SIX3, SOX9, GLIS3, FOXD1, ZNF92 , C11orf9 and combinations thereof directly via the dCas9 fusion protein.

In various embodiments, the method is free of the use of a gRNA specific to a target site that is/that is in proximity of a promoter region of: cMyc, Klf4, Nrl, Crx, Rax, LHX2, SIX3, SOX9, GLIS3, FOXD1, ZNF92, C11orf9 and combinations thereof.

In some examples, the method comprises modulating/promoting/enhancing/increasing the expression of (or activating) three transcription factors PAX6, MITF and OTX2, and optionally other transcription factors, wherein/whereby activation of the three transcription factors (sufficiently) drives differentiation of the cell e.g. into an RPE cell.

The method as described herein have been shown to be free of any supplement of growth factors (GFs) or small molecules such as activin A or retinoid acid together with Sonic Hedgehog (SHH). Thus, in some examples, the method does not comprise providing a growth factor e.g. an extrinsic growth factor (such as Activin A or Sonic hedgehog (SHH)), extrinsic transcription factors and/or small molecule (such as retinoic acid) e.g. to modulate the expression of one or more differentiation factors.

As illustrated in the Example section, the method as described herein does not include the step of inducing gene expression with a small molecule such as doxycycline. Thus, in some examples, the method does not comprise use of an inducible system such as a doxycycline inducible system. In some examples, the method is substantially reproducible. In some examples, the method is an in vivo, ex vivo or in vitro method.

In some examples, the method does not comprise introducing a whole (exogenous) nucleic acid, e.g. a whole cDNA, encoding the one or more differentiation factors into the cell, e.g. hESC3 cell or iPSC cell, to modulate the expression of one or more differentiation factors. Thus, in various embodiments, the method is free of exogenous growth factor, free of inducible system, and/or is free of whole exogenous nucleic acid. In various embodiments, wherein modulating the expression of one or more differentiation factors comprises an endogenous activation of the one or more differentiation factors. That is, the present disclosure relates to the use of CRISPR to activate endogenous genes to obtain differentiated cells. In various embodiments, wherein modulating the expression of one or more differentiation factors comprises a simultaneous activation of the one or more differentiation factors.

As illustrated in the Example section, the present disclosure also envisages a method of engineering an RPE cell. In various examples, the method may comprise a. providing CRISPR/dCas9-SAM expressing stable cells; b. providing an sgRNA lentivirus that targets PAX6, an sgRNA lentivirus that targets MITF, and an sgRNA lentivirus that targets OTX2; c. transducing the CRISPR/dCas9-SAM expressing stable cells using a mixture comprising the sgRNA lentivirus that targets PAX6, the sgRNA lentivirus that targets MITF, and the sgRNA lentivirus that targets OTX2 thereby obtaining transduced CRISPR/dCas9-SAM expressing stable cells; and d. maintaining the transduced CRISPR/dCas9-SAM expressing stable cells in RPE maintenance medium (RPEM) thereby obtaining RPE cells.

In some examples, step a. may further comprise providing hESC3 or iPSC cells; transducing the cells using lentiviral vectors dCas9-VP64 and MS2-p65-HSF1 thereby obtaining transduced hESC3 or iPSC cells; and performing antibiotic selection (e.g. using Hygromycyn and Blasticidin) thereby obtaining the CRISPR/dCas9-SAM expressing stable cells.

In some examples, step b. may further comprise providing HEK293T cells; transfecting the cells using a lentiviral expression vector comprising an sgRNA as described herein, harvesting a virus supernatant; and concentrating the virus supernatant thereby obtaining the sgRNA lentivirus that targets PAX6, the sgRNA lentivirus that targets MITF, and the sgRNA lentivirus that targets OTX2.

Also disclosed is an engineered RPE cell or cell lines of the present disclosure. In some examples, a cell/cell line/human cell/specialized cell/engineered cell/RPE cell/RPE cell line/RPE cell population/RPE sheet produced by the method as described herein, or progenies thereof.

Also disclosed is a cell population/sheet produced by the method of any of the preceding AS, or progenies thereof, wherein the cell population/sheet is substantially homogenous.

Also disclosed is a cell/cell line/stem cell/progenitor cell/precursor cell/human cell/specialized cell/engineered cell/RPE cell/RPE cell line/RPE cell population/RPE sheet, comprising/expressing a dCas9 fusion protein that is configured to modulate the expression of one or more differentiation factors, the dCas9 fusion protein comprising dCas9 and an effector, or progenies thereof. In some examples, the cell/cell line/cell population/sheet may further comprising/expressing a guide RNA (gRNA), optionally a single/short guide RNA (sgRNA), wherein the gRNA is capable of guiding the dCas9 fusion protein to a target site that is/that is in proximity of the promoter region of the one or more differentiation factors to allow the dCas9 fusion protein to modulate the expression of the one or more differentiation factors. In some examples, the cell/cell line/cell population/sheet further comprising/expressing a plurality of gRNA, the plurality of gRNA being specific to different target sites, optionally wherein the amount of each gRNA in the plurality of gRNA is substantially the same.

In some examples, the cell/cell line/cell population/sheet further comprising/expressing an activator module comprising an RNA-binding protein capable of binding to the gRNA, optionally wherein the RNA-binding protein comprises MS2 coat protein (MCP).

In some examples, the activator module further comprises one or more transcriptional regulators. In some examples, the cell/cell line/cell population/sheet comprising/expressing a CRISPR/dCas9 synergistic activation mediators (CRISPR/dCas9-SAM) complex/dCas9 ribonucleoprotein complex (e.g. a complex comprising the dCas9 fusion protein).

Also disclosed is a cell comprising a dCas9 fusion protein that is configured to modulate the expression of one or more differentiation factors, the dCas9 fusion protein comprising dCas9 and an effector, or progenies thereof. In some examples, the cell may comprise a guide RNA (gRNA) capable of guiding the dCas9 fusion protein to a target site that is/that is in proximity of the promoter region of the one or more differentiation factors to allow the dCas9 fusion protein to modulate the expression of the one or more differentiation factors.

Also disclosed is a cell having a second differentiation status (or its progenies thereof) that was differentiated from a cell having a first differentiation status, wherein the cell having the first differentiation status comprises a dCas9 fusion protein that is configured to modulate the expression of one or more differentiation factors, the dCas9 fusion protein comprising dCas9 and an effector.

In some examples, the cell having the first differentiation status comprises one or more features of the cell described hereinbefore. In some examples, the cell having the second first differentiation status is a RPE cell and the cell having the first differentiation status is a stem cell. In some examples, the cell having the second first differentiation status has one of more of the following characteristics as compared to the cell having the first differentiation status (at e.g. Day 4, 10, 18 or 28 post transfection/transduction):
a) reduced expression of OCT4-;
b) increased expression of PAX6;
c) increased expression of MITF;
d) increased expression of OTX2;
e) increased expression of LHX2;
f) increased expression of RAX;
g) increased expression of Tyrosinase;
h) increased expression of pMEL17;
i) increased expression of Tyrp1;
j) increased expression of Tyrp2;
k) increased expression of CRALBP;
l) increased expression of RPE65;
m) increased expression of BEST1; and
n) increased expression of PEDF.

In various embodiments, the cell having the second differentiation status is devoid of a dCas9 fusion protein or a CRISPR/dCas9-SAM complex. That is, the cell having the second differentiation status is devoid of/does not comprise/does not express a dCas9 fusion protein or a CRISPR/dCas9-SAM complex.

Also disclosed is a cell/cell line/stem cell/human cell/specialized cell/engineered cell/RPE cell/RPE cell line/RPE cell population/RPE sheet (or progenies thereof) that was transfected/transduced with a nucleic acid encoding a dCas9 fusion protein that is configured to modulate the expression of one or more differentiation factors, the dCas9 fusion protein comprising dCas9 and an effector (optionally the cell/cell line/population/sheet including/comprising a cell/cell line/population/sheet that was transfected/transduced with a nucleic acid encoding a dCas9 fusion protein but does not (presently) express the dCas9 fusion protein).

Also disclosed is a cell/cell line/stem cell/human cell/specialized cell/engineered cell/RPE cell/RPE cell line/RPE cell population/RPE sheet/progenies thereof of any of the preceding AS, wherein the cell/cell line/cell population/sheet was further transfected/transduced with a nucleic acid transcribing a gRNA, optionally a sgRNA, that is capable of guiding the dCas9 fusion protein to a target site that is/that is in proximity of the promoter region of the one or more differentiation factors to allow the dCas9 fusion protein to modulate the expression of the one or more differentiation factors (optionally the cell/cell line/population/sheet including/comprising a cell/cell line/population/sheet that was transfected/transduced with a nucleic acid transcribing a gRNA but does not (presently) express the gRNA).

Also disclosed is a cell/cell line/stem cell/human cell/specialized cell/engineered cell/RPE cell/RPE cell line/RPE cell population/RPE sheet/progenies thereof of any of the preceding AS, wherein the cell/cell line/population/sheet was further transfected/transduced with a nucleic acid encoding an activator module, optionally wherein the nucleic acid encodes MCP, HSF1 and/or p65 (optionally the cell/cell line/population/sheet including/comprising a cell/cell line/population/sheet that was transfected/transduced with a nucleic acid encoding an activator module but does not (presently) express the activator module).

Also disclosed is a cell/cell line/stem cell/human cell/specialized cell/engineered cell/RPE cell/RPE cell line/RPE cell population/RPE sheet (or progenies thereof) that was transfected/transduced with nucleic acid(s) encoding a CRISPR/dCas9 synergistic activation mediators (CRISPR/dCas9-SAM) complex/dCas9 ribonucleoprotein complex (e.g. a complex comprising the dCas9 fusion protein) (optionally the cell/cell line/population/sheet including/comprising a cell/cell line/population/sheet that was transfected/transduced with nucleic acid(s) encoding a CRISPR/dCas9 synergistic activation mediators (CRISPR/dCas9-SAM) complex/dCas9 ribonucleoprotein complex (e.g. a complex comprising the dCas9 fusion protein) but does not (presently) express the activator module).

Also disclosed is a nucleic acid construct/expression construct/expression vector/plasmid/viral vector/recombinant construct comprising sequences encoding a dCas9 fusion protein that is configured to modulate the expression of one or more differentiation factors, the dCas9 fusion protein comprising dCas9 and an effector.

Also disclosed is a nucleic acid construct/expression construct/expression vector/plasmid/viral vector/recombinant construct comprising sequences transcribing a gRNA, optionally a sgRNA, that is capable of guiding a dCas9 fusion protein to a target site that is/that is in proximity of the promoter region of one or more differentiation factors to allow the dCas9 fusion protein to modulate the expression of the one or more differentiation factors, optionally wherein the nucleic acid construct/expression construct/expression vector/plasmid/viral vector/recombinant construct further comprises sequences encoding the dCas9 fusion protein, further optionally wherein the nucleic acid construct/expression construct/expression vector/plasmid/viral vector/recombinant construct comprises sequences transcribing a plurality of gRNAs (e.g. three gRNAs/sgRNAs).

Also disclosed is a nucleic acid construct/expression construct/expression vector/plasmid/viral vector/recombinant construct comprising sequences encoding an activator module, optionally wherein the nucleic acid encodes MCP, HSF1 and/or p65, further optionally wherein the nucleic acid construct/expression construct/expression vector/plasmid/viral vector/recombinant construct further comprises sequences encoding a dCas9 fusion protein and/or a gRNA.

Also disclosed is a nucleic acid construct/expression construct/expression vector/plasmid/viral vector/recombinant construct comprising sequences encoding a CRISPR/dCas9 synergistic activation mediators (CRISPR/dCas9-SAM) complex/dCas9 ribonucleoprotein complex (e.g. a complex comprising the dCas9 fusion protein).

Also disclosed is a guide RNA (gRNA) that is configured to guide a dCas9 fusion protein/CRISPR/dCas9 synergistic activation mediators (CRISPR/dCas9-SAM) complex/dCas9 ribonucleoprotein complex (e.g. a complex comprising the dCas9 fusion protein) to a target site/target genomic locus that is/that is in proximity of the promoter region of one or more differentiation factors that e.g. influence cell differentiation, cell dedifferentiation or cell transdifferentiation to e.g. allow the dCas9 fusion protein to modulate the expression of the one or more differentiation factors.

As used herein, the term “guide RNA (gRNA)” refers to a guide RNA which is a fusion protein between the gRNA guide sequence (crRNA) and the Cas9 recognition sequence (tracrRNA). It provides both targeting specificity and scaffolding or binding ability for Cas9 nuclease or nickase.

In some examples, the gRNA comprising a CRISPR RNA (crRNA) component/sequences and a transactivating CRISPR RNA (tracrRNA) component/sequences. In some examples, the gRNA comprising a stem-loop/hairpin structure, optionally a MS2 stem-loop/hairpin structure.

In some examples, at least a portion of the gRNA is capable of binding to the dCas9 fusion protein/CR ISPR/dCas9 synergistic activation mediators (CRISPR/dCas9-SAM) complex/dCas9 ribonucleoprotein complex (e.g. a complex comprising the dCas9 fusion protein).

In some examples, at least a portion of the guide RNA is capable of binding to the target site/target genomic locus. In some examples, at least a portion of the guide RNA is substantially complementary to (e.g. having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 99%, at least about 99%, or at least 100% identity with) the sequences of the target site/target genomic locus. In some examples, the target site/target genomic locus precede, optionally immediately precede, a 5′-NGG, 5′-NNGRRT, 5′-NNGRR(N), 5′-NNNNGATT, 5′-NNAGAAW, 5′-NAAAAC, 5′-NNGRRT, 5′-NNNACA and/or NNVRYM protospacer adjacent motif, wherein N=A or G or T or C, R=A or G, W=A or T, V=G or C or A, Y=C or T, and M=A or C).

In one aspect, there is provided a guide RNA (gRNA) to a target site that is or that is in proximity of the promoter region of one or more differentiation factors to modulate the expression of the one or more differentiation factors, wherein the gRNA is configured to guide a fusion protein selected from the group consisting of dCas9 fusion protein, CRISPR/dCas9 synergistic activation mediators (CRISPR/dCas9-SAM) complex, dCas9 ribonucleoprotein complex, dCas9-VP64, dCas9-VPR, dCas9-VP64, and MS2-P65-HSF1.

In some examples, at least a portion of the guide RNA is capable of binding to the target site/target genomic locus that is in an about −300 base pairs (bp) to about +5 bp window, an about −250 bp to about +3 bp window or an about −200 bp to about +1 bp window of the promoter region of one or more differentiation factors. In some examples, the one or more differentiation factors is selected from the group consisting of PAX6, MITF, OTX2, EPO, SCF, TPO, GM-CSF, G-CSF, and combinations thereof.

In various embodiments, at least a portion of the guide RNA is capable of binding to the target site/target genomic locus that is in an about −300 base pairs (bp) to about +5 bp window of the promoter region of one or more differentiation factors selected from the group consisting of PAX6, MITF, OTX2 and combinations thereof.

In various embodiments, at least a portion of the guide RNA is capable of binding to the target site/target genomic locus that is in an about −300 base pairs (bp) to about +5 bp window of the promoter region of one or more differentiation factors selected from the group consisting of , EPO, SCF, TPO, GM-CSF, G-CSF and combinations thereof.

In some examples, the gRNA is capable of guiding a dCas9 fusion protein/CRISPR/dCas9 synergistic activation mediators (CRISPR/dCas9-SAM) complex/dCas9 ribonucleoprotein complex (e.g. a complex comprising the dCas9 fusion protein) to a target site/target genomic locus that is/that is in proximity of the promoter region of PAX6, and inducing at least about 1-fold change, at least about 2-fold change, at least about 3-fold change, at least about 4-fold change, at least about 5-fold change, at least about 30-fold change, at least about 31-fold change, at least about 32-fold change, at least about 33-fold change, at least about 34-fold change, at least about 35-fold change, at least about 36-fold change, at least about 37-fold change, at least about 38-fold change, at least about 39-foldchange, at least about 40-fold change, at least about 41-fold change, at least about 42-fold change, at least about 43-fold change, at least about 44-fold change, at least about 45-fold change, at least about 46-fold change, at least about 47-fold change, at least about 48-fold change, at least about 49 fold-change, at least about 50-fold change, at least about 51-fold change, at least about 52-fold change, at least about 53-fold change, at least about 54-fold change, at least about 55-fold change, at least about 56-fold change, at least about 57-fold change, at least about 58-fold change, at least about 59 fold-change, at least about 60-fold change, at least about 100-fold change, at least about 200-fold change, at least about 300-fold change, at least about 1500-fold change, 1600-fold change, at least about 1700-fold change, at least about 1800-fold change, at least about 1900-fold change, at least about 2000-fold change, 2100-fold change, 2200-fold change, at least about 2300-fold change, at least about 2400-fold change, at least about 2500-fold change, at least about 2600-fold change, 2700-fold change, at least about 2800-fold change, at least about 2900-fold change, at least about 3000-fold change, at least about 150000-fold change, 160000-fold change, at least about 170000-fold change, at least about 180000-fold change, at least about 190000-fold change, at least about 200000-fold change, 210000-fold change, 220000-fold change, at least about 230000-fold change, at least about 240000-fold change, at least about 250000-fold change, at least about 260000-fold change, 270000-fold change, at least about 280000-fold change, at least about 290000-fold change or at least about 300000-fold change in the expression of PAX6 (e.g. a fold change in an amount of PAX6 mRNA after normalization by GAPDH and standardization to a control sample about 4 days post transfection/transduction, as measured by qRT-PCR analysis), optionally wherein the gRNA is capable of inducing a higher fold change in the expression of the PAX6 (+5a) isoform as compared to the PAX6 (-5a) isoform.

In some examples, the gRNA is capable of guiding a dCas9 fusion protein/CRISPR/dCas9 synergistic activation mediators (CRISPR/dCas9-SAM) complex/dCas9 ribonucleoprotein complex (e.g. a complex comprising the dCas9 fusion protein) to a target site/target genomic locus that is/that is in proximity of the promoter region of PAX6, and inducing at least about 1-fold increase, at least about 2-fold increase, at least about 3-fold increase, at least about 4-fold increase, at least about 5-fold increase, at least about 30-fold increase, at least about 31-fold increase, at least about 32-fold increase, at least about 33-fold increase, at least about 34-fold increase, at least about 35-fold increase, at least about 36-fold increase, at least about 37-fold increase, at least about 38-fold increase, at least about 39 fold-increase, at least about 40-fold increase, at least about 41-fold increase, at least about 42-fold increase, at least about 43-fold increase, at least about 44-fold increase, at least about 45-fold increase, at least about 46-fold increase, at least about 47-fold increase, at least about 48-fold increase, at least about 49 fold-increase, at least about 50-fold increase, at least about 51-fold increase, at least about 52-fold increase, at least about 53-fold increase, at least about 54-fold increase, at least about 55-fold increase, at least about 56-fold increase, at least about 57-fold increase, at least about 58-fold increase, at least about 59 fold-increase, at least about 60-fold increase, at least about 100-fold increase, at least about 200-fold increase, at least about 300-fold increase, at least about 1500-fold increase, 1600-fold increase, at least about 1700-fold increase, at least about 1800-fold increase, at least about 1900-fold increase, at least about 2000-fold increase, 2100-fold increase, 2200-fold increase, at least about 2300-fold increase, at least about 2400-fold increase, at least about 2500-fold increase, at least about 2600-fold increase, 2700-fold increase, at least about 2800-fold increase, at least about 2900-fold increase, at least about 3000-fold increase, at least about 150000-fold increase, 160000-fold increase, at least about 170000-fold increase, at least about 180000-fold increase, at least about 190000-fold increase, at least about 200000-fold increase, 210000-fold increase, 220000-fold increase, at least about 230000-fold increase, at least about 240000-fold increase, at least about 250000-fold increase, at least about 260000-fold increase, 270000-fold increase, at least about 280000-fold increase, at least about 290000-fold increase or at least about 300000-fold increase in the expression of PAX6 (e.g. a fold increase in an amount of PAX6 mRNA after normalization by GAPDH and standardization to a control sample about 4 days post transfection/transduction, as measured by qRT-PCR analysis), optionally wherein the gRNA is capable of inducing a higher increase in the expression of the PAX6 (+5a) isoform as compared to the PAX6 (−5a) isoform.

In some examples, the gRNA is capable of guiding a dCas9 fusion protein/CRISPR/dCas9 synergistic activation mediators (CRISPR/dCas9-SAM) complex/dCas9 ribonucleoprotein complex (e.g. a complex comprising the dCas9 fusion protein) to a target site/target genomic locus that is/that is in proximity of the promoter region of MITF, and inducing at least about 5-fold change, at least about 10-fold change, at least about 15-fold change, at least about 20-fold change, at least about 25-fold change, at least about 30-fold change, at least about 35-fold change, at least about 40-fold change, at least about 45 fold-change, at least about 50-fold change, at least about 55-fold change, at least about 60-fold change, at least about 65-fold change, at least about 70-fold change, at least about 75-fold change, at least about 80-fold change, at least about 85-fold change, at least about 90-fold change, at least about 95 fold-change, at least about 100-fold change, at least about 105-fold change, at least about 110-fold change, at least about 115-fold change, at least about 120-fold change, at least about 125-fold change, at least about 130-fold change, at least about 135-fold change, at least about 140-fold change, at least about 145 fold-change, at least about 150-fold change, at least about 155 fold-change, at least about 160-fold change, at least about 165-fold change, at least about 170-fold change, at least about 175 fold-change, at least about 180-fold change, at least about 185 fold-change, at least about 190-fold change, at least about 195 fold-change, or at least about 200-fold change in the expression of MITF (e.g. a fold change in an amount of MITF mRNA after normalization by GAPDH and standardization to a control sample about 4 days post transfection/transduction, as measured by qRT-PCR analysis).

In some examples, the gRNA is capable of guiding a dCas9 fusion protein/CRISPR/dCas9 synergistic activation mediators (CRISPR/dCas9-SAM) complex/dCas9 ribonucleoprotein complex (e.g. a complex comprising the dCas9 fusion protein) to a target site/target genomic locus that is/that is in proximity of the promoter region of MITF, and inducing at least about 5-fold increase, at least about 10-fold increase, at least about 15-fold increase, at least about 20-fold increase, at least about 25-fold increase, at least about 30-fold increase, at least about 35-fold increase, at least about 40-fold increase, at least about 45 fold-increase, at least about 50-fold increase, at least about 55-fold increase, at least about 60-fold increase, at least about 65-fold increase, at least about 70-fold increase, at least about 75-fold increase, at least about 80-fold increase, at least about 85-fold increase, at least about 90-fold increase, at least about 95 fold-increase, at least about 100-fold increase, at least about 105-fold increase, at least about 110-fold increase, at least about 115-fold increase, at least about 120-fold increase, at least about 125-fold increase, at least about 130-fold increase, at least about 135-fold increase, at least about 140-fold increase, at least about 145 fold-increase, at least about 150-fold increase, at least about 155 fold-increase, at least about 160-fold increase, at least about 165-fold increase, at least about 170-fold increase, at least about 175 fold-increase, at least about 180-fold increase, at least about 185 fold-increase, at least about 190-fold increase, at least about 195 fold-increase, or at least about 200-fold increase in the expression of MITF (e.g. a fold increase in an amount of MITF mRNA after normalization by GAPDH and standardization to a control sample about 4 days post transfection/transduction, as measured by qRT-PCR analysis).

In some examples, the gRNA is capable of guiding a dCas9 fusion protein/CRISPR/dCas9 synergistic activation mediators (CRISPR/dCas9-SAM) complex/dCas9 ribonucleoprotein complex (e.g. a complex comprising the dCas9 fusion protein) to a target site/target genomic locus that is/that is in proximity of the promoter region of OTX2, and inducing at least about 1-fold change, at least about 2-fold change, at least about 3-fold change, at least about 4-fold change, at least about 5-fold change, at least about 1000-fold change, at least about 2000-fold change, at least about 3000-fold change, at least about 4000-fold change, at least about 5000-fold change, at least about 30000-fold change, 31000-fold change, 32000-fold change, at least about 33000-fold change, at least about 34000-fold change, at least about 35000-fold change, at least about 36000-fold change, 37000-fold change, at least about 38000-fold change, at least about 39000-fold change or at least about 40000-fold change in the expression of OTX2 (e.g. a fold change in an amount of OTX2 mRNA after normalization by GAPDH and standardization to a control sample about 4 days post transfection/transduction, as measured by qRT-PCR analysis).

In some examples, the gRNA is capable of guiding a dCas9 fusion protein/CRISPR/dCas9 synergistic activation mediators (CRISPR/dCas9-SAM) complex/dCas9 ribonucleoprotein complex (e.g. a complex comprising the dCas9 fusion protein) to a target site/target genomic locus that is/that is in proximity of the promoter region of OTX2, and inducing at least about 1-fold increase, at least about 2-fold increase, at least about 3-fold increase, at least about 4-fold increase, at least about 5-fold increase, at least about 1000-fold increase, at least about 2000-fold increase, at least about 3000-fold increase, at least about 4000-fold change, at least about 5000-fold change, at least about 30000-fold increase, 31000-fold increase, 32000-fold increase, at least about 33000-fold increase, at least about 34000-fold increase, at least about 35000-fold increase, at least about 36000-fold increase, 37000-fold increase, at least about 38000-fold increase, at least about 39000-fold increase or at least about 40000-fold increase in the expression of OTX2 (e.g. a fold increase in an amount of OTX2 mRNA after normalization by GAPDH and standardization to a control sample about 4 days post transfection/transduction, as measured by qRT-PCR analysis).

In some examples, the gRNA is capable of guiding a dCas9 fusion protein/CRISPR/dCas9 synergistic activation mediators (CRISPR/dCas9-SAM) complex/dCas9 ribonucleoprotein complex (e.g. a complex comprising the dCas9 fusion protein) to a target site/target genomic locus that is/that is in proximity of the promoter region of EPO, and inducing at least about 1-fold change, at least about 2-fold change, at least about 3-fold change, at least about 4-fold change, at least about 5-fold change, at least about 10-fold change, at least about 15-fold change, at least about 20-fold change, at least about 25-fold change, at least about 30-fold change, at least about 31-fold change, at least about 32-fold change, at least about 33-fold change, at least about 34-fold change, at least about 35-fold change, at least about 36-fold change, at least about 37-fold change, at least about 38-fold change, at least about 39-fold change, at least about 40-fold change, at least about 41-fold change, at least about 42-fold change, at least about 43-fold change, at least about 44-fold change, at least about 45-fold change, at least about 46-fold change, at least about 47-fold change, at least about 48-fold change, at least about 49 fold-change, at least about 50-fold change, at least about 51-fold change, at least about 52-fold change, at least about 53-fold change, at least about 54-fold change, at least about 55-fold change, at least about 56-fold change, at least about 57-fold change, at least about 58-fold change, at least about 59 fold-change, at least about 60-fold change, at least about 100-fold change, at least about 200-fold change, at least about 300-fold change, at least about 1000-fold change, at least about 2000-fold change, at least about 3000-fold change, at least about 4000-fold change, at least about 5000-fold change, at least about 30000-fold change, 31000-fold change, 32000-fold change, at least about 33000-fold change, at least about 34000-fold change, at least about 35000-fold change, at least about 36000-fold change, 37000-fold change, at least about 38000-fold change, at least about 39000-fold change or at least about 40000-fold change in the expression of EPO (e.g. a fold change in an amount of EPO mRNA after normalization by GAPDH and standardization to a control sample about 3 or 4 days post transfection/transduction, as measured by qRT-PCR analysis).

In some examples, the gRNA is capable of guiding a dCas9 fusion protein/CRISPR/dCas9 synergistic activation mediators (CRISPR/dCas9-SAM) complex/dCas9 ribonucleoprotein complex (e.g. a complex comprising the dCas9 fusion protein) to a target site/target genomic locus that is/that is in proximity of the promoter region of SCF, and inducing at least about 1-fold change, at least about 2-fold change, at least about 3-fold change, at least about 4-fold change, at least about 5-fold change, at least about 10-fold change, at least about 15-fold change, at least about 20-fold change, at least about 25-fold change, at least about 30-fold change, at least about 31-fold change, at least about 32-fold change, at least about 33-fold change, at least about 34-fold change, at least about 35-fold change, at least about 36-fold change, at least about 37-fold change, at least about 38-fold change, at least about 39-fold change, at least about 40-fold change, at least about 41-fold change, at least about 42-fold change, at least about 43-fold change, at least about 44-fold change, at least about 45-fold change, at least about 46-fold change, at least about 47-fold change, at least about 48-fold change, at least about 49 fold-change, at least about 50-fold change, at least about 51-fold change, at least about 52-fold change, at least about 53-fold change, at least about 54-fold change, at least about 55-fold change, at least about 56-fold change, at least about 57-fold change, at least about 58-fold change, at least about 59 fold-change, at least about 60-fold change, at least about 100-fold change, at least about 200-fold change, at least about 300-fold change, at least about 1000-fold change, at least about 2000-fold change, at least about 3000-fold change, at least about 4000-fold change, at least about 5000-fold change, at least about 30000-fold change, 31000-fold change, 32000-fold change, at least about 33000-fold change, at least about 34000-fold change, at least about 35000-fold change, at least about 36000-fold change, 37000-fold change, at least about 38000-fold change, at least about 39000-fold change or at least about 40000-fold change in the expression of SCF (e.g. a fold change in an amount of SCF mRNA after normalization by GAPDH and standardization to a control sample about 3 or 4 days post transfection/transduction, as measured by qRT-PCR analysis).

In some examples, the gRNA is capable of guiding a dCas9 fusion protein/CRISPR/dCas9 synergistic activation mediators (CRISPR/dCas9-SAM) complex/dCas9 ribonucleoprotein complex (e.g. a complex comprising the dCas9 fusion protein) to a target site/target genomic locus that is/that is in proximity of the promoter region of TPO, and inducing at least about 0.1-fold change, at least about 0.2-fold change, at least about 0.3-fold change, at least about 0.4-fold change, at least about 0.5-fold change, at least about 0.6-fold change, at least about 0.7-fold change, at least about 0.8-fold change, at least about 0.9-fold change, at least about 1-fold change, at least about 1.1-fold change, at least about 1.2-fold change, at least about 1.3-fold change, at least about 1.4-fold change, at least about 1.5-fold change, at least about 1.6-fold change, at least about 1.7-fold change, at least about 1.8-fold change, at least about 1.9-fold change, at least about 2-fold change, at least about 2.1-fold change, at least about 2.2-fold change, at least about 2.3-fold change, at least about 2.4-fold change, at least about 2.5-fold change, at least about 2.6-fold change, at least about 2.7-fold change, at least about 2.8-fold change, at least about 2.9-fold change, at least about 3-fold change, at least about 3.5-fold change, at least about 4-fold change, at least about 4.5-fold change, at least about 5-fold change, at least about 5.5-fold change, at least about 10-fold change, at least about 15-fold change, at least about 20-fold change, at least about 25-fold change, at least about 30-fold change, at least about 31-fold change, at least about 32-fold change, at least about 33-fold change, at least about 34-fold change, at least about 35-fold change, at least about 36-fold change, at least about 37-fold change, at least about 38-fold change, at least about 39-fold change, at least about 40-fold change, at least about 41-fold change, at least about 42-fold change, at least about 43-fold change, at least about 44-fold change, at least about 45-fold change, at least about 46-fold change, at least about 47-fold change, at least about 48-fold change, at least about 49 fold-change, at least about 50-fold change, at least about 51-fold change, at least about 52-fold change, at least about 53-fold change, at least about 54-fold change, at least about 55-fold change, at least about 56-fold change, at least about 57-fold change, at least about 58-fold change, at least about 59 fold-change, at least about 60-fold change, at least about 100-fold change, at least about 200-fold change, at least about 300-fold change, at least about 1000-fold change, at least about 2000-fold change, at least about 3000-fold change, at least about 4000-fold change, at least about 5000-fold change, at least about 30000-fold change, 31000-fold change, 32000-fold change, at least about 33000-fold change, at least about 34000-fold change, at least about 35000-fold change, at least about 36000-fold change, 37000-fold change, at least about 38000-fold change, at least about 39000-fold change or at least about 40000-fold change in the expression of TPO (e.g. a fold change in an amount of TPO mRNA after normalization by GAPDH and standardization to a control sample about 3 or 4 days post transfection/transduction, as measured by qRT-PCR analysis).

In some examples, the gRNA is capable of guiding a dCas9 fusion protein/CRISPR/dCas9 synergistic activation mediators (CRISPR/dCas9-SAM) complex/dCas9 ribonucleoprotein complex (e.g. a complex comprising the dCas9 fusion protein) to a target site/target genomic locus that is/that is in proximity of the promoter region of GM-CSF, and inducing at least about 0.1-fold change, at least about 0.2-fold change, at least about 0.3-fold change, at least about 0.4-fold change, at least about 0.5-fold change, at least about 0.6-fold change, at least about 0.7-fold change, at least about 0.8-fold change, at least about 0.9-fold change, at least about 1-fold change, at least about 1.1-fold change, at least about 1.2-fold change, at least about 1.3-fold change, at least about 1.4-fold change, at least about 1.5-fold change, at least about 1.6-fold change, at least about 1.7-fold change, at least about 1.8-fold change, at least about 1.9-fold change, at least about 2-fold change, at least about 2.1-fold change, at least about 2.2-fold change, at least about 2.3-fold change, at least about 2.4-fold change, at least about 2.5-fold change, at least about 2.6-fold change, at least about 2.7-fold change, at least about 2.8-fold change, at least about 2.9-fold change, at least about 3-fold change, at least about 3.5-fold change, at least about 4-fold change, at least about 4.5-fold change, at least about 5-fold change, at least about 5.5-fold change, at least about 10-fold change, at least about 15-fold change, at least about 20-fold change, at least about 25-fold change, at least about 30-fold change, at least about 31-fold change, at least about 32-fold change, at least about 33-fold change, at least about 34-fold change, at least about 35-fold change, at least about 36-fold change, at least about 37-fold change, at least about 38-fold change, at least about 39-fold change, at least about 40-fold change, at least about 41-fold change, at least about 42-fold change, at least about 43-fold change, at least about 44-fold change, at least about 45-fold change, at least about 46-fold change, at least about 47-fold change, at least about 48-fold change, at least about 49 fold-change, at least about 50-fold change, at least about 51-fold change, at least about 52-fold change, at least about 53-fold change, at least about 54-fold change, at least about 55-fold change, at least about 56-fold change, at least about 57-fold change, at least about 58-fold change, at least about 59 fold-change, at least about 60-fold change, at least about 100-fold change, at least about 200-fold change, at least about 300-fold change, at least about 1000-fold change, at least about 2000-fold change, at least about 3000-fold change, at least about 4000-fold change, at least about 5000-fold change, at least about 30000-fold change, 31000-fold change, 32000-fold change, at least about 33000-fold change, at least about 34000-fold change, at least about 35000-fold change, at least about 36000-fold change, 37000-fold change, at least about 38000-fold change, at least about 39000-fold change or at least about 40000-fold change in the expression of GM-CSF (e.g. a fold change in an amount of GM-CSF mRNA after normalization by GAPDH and standardization to a control sample about 3 or 4 days post transfection/transduction, as measured by qRT-PCR analysis).

In some examples, the gRNA is capable of guiding a dCas9 fusion protein/CRISPR/dCas9 synergistic activation mediators (CRISPR/dCas9-SAM) complex/dCas9 ribonucleoprotein complex (e.g. a complex comprising the dCas9 fusion protein) to a target site/target genomic locus that is/that is in proximity of the promoter region of G-CSF, and inducing at least about 1-fold change, at least about 2-fold change, at least about 3-fold change, at least about 4-fold change, at least about 5-fold change, at least about 1000-fold change, at least about 2000-fold change, at least about 3000-fold change, at least about 4000-fold change, at least about 5000-fold change, at least about 30000-fold change, 31000-fold change, 32000-fold change, at least about 33000-fold change, at least about 34000-fold change, at least about 35000-fold change, at least about 36000-fold change, 37000-fold change, at least about 38000-fold change, at least about 39000-fold change or at least about 40000-fold change in the expression of G-CSF (e.g. a fold change in an amount of G-CSF mRNA after normalization by GAPDH and standardization to a control sample about 3 or 4 days post transfection/transduction, as measured by qRT-PCR analysis).

In some examples, the gRNA is capable of guiding a dCas9 fusion protein/CRISPR/dCas9 synergistic activation mediators (CRISPR/dCas9-SAM) complex/dCas9 ribonucleoprotein complex (e.g. a complex comprising the dCas9 fusion protein) to a target site/target genomic locus that is/that is in proximity of the promoter region of EPO, and inducing at least about 1-fold increase, at least about 2-fold increase, at least about 3-fold increase, at least about 4-fold increase, at least about 5-fold increase, at least about 30-fold increase, at least about 31-fold increase, at least about 32-fold increase, at least about 33-fold increase, at least about 34-fold increase, at least about 35-fold increase, at least about 36-fold increase, at least about 37-fold increase, at least about 38-fold increase, at least about 39 fold-increase, at least about 40-fold increase, at least about 41-fold increase, at least about 42-fold increase, at least about 43-fold increase, at least about 44-fold increase, at least about 45-fold increase, at least about 46-fold increase, at least about 47-fold increase, at least about 48-fold increase, at least about 49 fold-increase, at least about 50-fold increase, at least about 51-fold increase, at least about 52-fold increase, at least about 53-fold increase, at least about 54-fold increase, at least about 55-fold increase, at least about 56-fold increase, at least about 57-fold increase, at least about 58-fold increase, at least about 59 fold-increase, at least about 60-fold increase, at least about 100-fold increase, at least about 200-fold increase, at least about 300-fold increase, at least about 1500-fold increase, 1600-fold increase, at least about 1700-fold increase, at least about 1800-fold increase, at least about 1900-fold increase, at least about 2000-fold increase, 2100-fold increase, 2200-fold increase, at least about 2300-fold increase, at least about 2400-fold increase, at least about 2500-fold increase, at least about 2600-fold increase, 2700-fold increase, at least about 2800-fold increase, at least about 2900-fold increase, at least about 3000-fold increase, at least about 150000-fold increase, 160000-fold increase, at least about 170000-fold increase, at least about 180000-fold increase, at least about 190000-fold increase, at least about 200000-fold increase, 210000-fold increase, 220000-fold increase, at least about 230000-fold increase, at least about 240000-fold increase, at least about 250000-fold increase, at least about 260000-fold increase, 270000-fold increase, at least about 280000-fold increase, at least about 290000-fold increase or at least about 300000-fold increase in the expression of EPO (e.g. a fold increase in an amount of EPO mRNA after normalization by GAPDH and standardization to a control sample about 3 or 4 days post transfection/transduction, as measured by qRT-PCR analysis).

In some examples, the gRNA is capable of guiding a dCas9 fusion protein/CRISPR/dCas9 synergistic activation mediators (CRISPR/dCas9-SAM) complex/dCas9 ribonucleoprotein complex (e.g. a complex comprising the dCas9 fusion protein) to a target site/target genomic locus that is/that is in proximity of the promoter region of SCF, and inducing at least about 1-fold increase, at least about 2-fold increase, at least about 3-fold increase, at least about 4-fold increase, at least about 5-fold increase, at least about 30-fold increase, at least about 31-fold increase, at least about 32-fold increase, at least about 33-fold increase, at least about 34-fold increase, at least about 35-fold increase, at least about 36-fold increase, at least about 37-fold increase, at least about 38-fold increase, at least about 39 fold-increase, at least about 40-fold increase, at least about 41-fold increase, at least about 42-fold increase, at least about 43-fold increase, at least about 44-fold increase, at least about 45-fold increase, at least about 46-fold increase, at least about 47-fold increase, at least about 48-fold increase, at least about 49 fold-increase, at least about 50-fold increase, at least about 51-fold increase, at least about 52-fold increase, at least about 53-fold increase, at least about 54-fold increase, at least about 55-fold increase, at least about 56-fold increase, at least about 57-fold increase, at least about 58-fold increase, at least about 59 fold-increase, at least about 60-fold increase, at least about 100-fold increase, at least about 200-fold increase, at least about 300-fold increase, at least about 1500-fold increase, 1600-fold increase, at least about 1700-fold increase, at least about 1800-fold increase, at least about 1900-fold increase, at least about 2000-fold increase, 2100-fold increase, 2200-fold increase, at least about 2300-fold increase, at least about 2400-fold increase, at least about 2500-fold increase, at least about 2600-fold increase, 2700-fold increase, at least about 2800-fold increase, at least about 2900-fold increase, at least about 3000-fold increase, at least about 150000-fold increase, 160000-fold increase, at least about 170000-fold increase, at least about 180000-fold increase, at least about 190000-fold increase, at least about 200000-fold increase, 210000-fold increase, 220000-fold increase, at least about 230000-fold increase, at least about 240000-fold increase, at least about 250000-fold increase, at least about 260000-fold increase, 270000-fold increase, at least about 280000-fold increase, at least about 290000-fold increase or at least about 300000-fold increase in the expression of SCF (e.g. a fold increase in an amount of SCF mRNA after normalization by GAPDH and standardization to a control sample about 3 or 4 days post transfection/transduction, as measured by qRT-PCR analysis).

In some examples, the gRNA is capable of guiding a dCas9 fusion protein/CRISPR/dCas9 synergistic activation mediators (CRISPR/dCas9-SAM) complex/dCas9 ribonucleoprotein complex (e.g. a complex comprising the dCas9 fusion protein) to a target site/target genomic locus that is/that is in proximity of the promoter region of TPO, and inducing at least about 0.1-fold increase, at least about 0.2-fold increase, at least about 0.3-fold increase, at least about 0.4-fold increase, at least about 0.5-fold increase, at least about 0.6-fold increase, at least about 0.7-fold increase, at least about 0.8-fold increase, at least about 0.9-fold increase, at least about 1-fold increase, at least about 1.1-fold increase, at least about 1.2-fold increase, at least about 1.3-fold increase, at least about 1.4-fold increase, at least about 1.5-fold increase, at least 1.6-fold increase, at least 1.7-fold increase, at least 1.8-fold increase, at least 1.9-fold increase, at least about 2-fold increase, at least about 2.1-fold increase, at least about 2.2-fold increase, at least about 2.3-fold increase, at least about 2.4-fold increase, at least about 2.5-fold increase, at least about 2.6-fold increase, at least about 2.7-fold increase, at least about 2.8-fold increase, at least about 2.9-fold increase, at least about 3-fold increase, at least about 3.5-fold increase, at least about 4-fold increase, at least about 4.5-fold increase, at least about 5-fold increase, at least about 10-fold increase, at least about 15-fold increase, at least about 20-fold increase, at least about 25-fold increase, at least about 30-fold increase, at least about 31-fold increase, at least about 32-fold increase, at least about 33-fold increase, at least about 34-fold increase, at least about 35-fold increase, at least about 36-fold increase, at least about 37-fold increase, at least about 38-fold increase, at least about 39 fold-increase, at least about 40-fold increase, at least about 41-fold increase, at least about 42-fold increase, at least about 43-fold increase, at least about 44-fold increase, at least about 45-fold increase, at least about 46-fold increase, at least about 47-fold increase, at least about 48-fold increase, at least about 49 fold-increase, at least about 50-fold increase, at least about 51-fold increase, at least about 52-fold increase, at least about 53-fold increase, at least about 54-fold increase, at least about 55-fold increase, at least about 56-fold increase, at least about 57-fold increase, at least about 58-fold increase, at least about 59 fold-increase, at least about 60-fold increase, at least about 100-fold increase, at least about 200-fold increase, at least about 300-fold increase, at least about 1500-fold increase, 1600-fold increase, at least about 1700-fold increase, at least about 1800-fold increase, at least about 1900-fold increase, at least about 2000-fold increase, 2100-fold increase, 2200-fold increase, at least about 2300-fold increase, at least about 2400-fold increase, at least about 2500-fold increase, at least about 2600-fold increase, 2700-fold increase, at least about 2800-fold increase, at least about 2900-fold increase, at least about 3000-fold increase, at least about 150000-fold increase, 160000-fold increase, at least about 170000-fold increase, at least about 180000-fold increase, at least about 190000-fold increase, at least about 200000-fold increase, 210000-fold increase, 220000-fold increase, at least about 230000-fold increase, at least about 240000-fold increase, at least about 250000-fold increase, at least about 260000-fold increase, 270000-fold increase, at least about 280000-fold increase, at least about 290000-fold increase or at least about 300000-fold increase in the expression of TPO (e.g. a fold increase in an amount of TPO mRNA after normalization by GAPDH and standardization to a control sample about 3 or 4 days post transfection/transduction, as measured by qRT-PCR analysis).

In some examples, the gRNA is capable of guiding a dCas9 fusion protein/CRISPR/dCas9 synergistic activation mediators (CRISPR/dCas9-SAM) complex/dCas9 ribonucleoprotein complex (e.g. a complex comprising the dCas9 fusion protein) to a target site/target genomic locus that is/that is in proximity of the promoter region of GM-CSF, and inducing at least about 0.1-fold increase, at least about 0.2-fold increase, at least about 0.3-fold increase, at least about 0.4-fold increase, at least about 0.5-fold increase, at least about 0.6-fold increase, at least about 0.7-fold increase, at least about 0.8-fold increase, at least about 0.9-fold increase, at least about 1-fold increase, at least about 1.1-fold increase, at least about 1.2-fold increase, at least about 1.3-fold increase, at least about 1.4-fold increase, at least about 1.5-fold increase, at least 1.6-fold increase, at least 1.7-fold increase, at least 1.8-fold increase, at least 1.9-fold increase, at least about 2-fold increase, at least about 2.1-fold increase, at least about 2.2-fold increase, at least about 2.3-fold increase, at least about 2.4-fold increase, at least about 2.5-fold increase, at least about 2.6-fold increase, at least about 2.7-fold increase, at least about 2.8-fold increase, at least about 2.9-fold increase, at least about 3-fold increase, at least about 3.5-fold increase, at least about 4-fold increase, at least about 4.5-fold increase, at least about 5-fold increase, at least about 10-fold increase, at least about 15-fold increase, at least about 20-fold increase, at least about 25-fold increase, at least about 30-fold increase, at least about 31-fold increase, at least about 32-fold increase, at least about 33-fold increase, at least about 34-fold increase, at least about 35-fold increase, at least about 36-fold increase, at least about 37-fold increase, at least about 38-fold increase, at least about 39 fold-increase, at least about 40-fold increase, at least about 41-fold increase, at least about 42-fold increase, at least about 43-fold increase, at least about 44-fold increase, at least about 45-fold increase, at least about 46-fold increase, at least about 47-fold increase, at least about 48-fold increase, at least about 49 fold-increase, at least about 50-fold increase, at least about 51-fold increase, at least about 52-fold increase, at least about 53-fold increase, at least about 54-fold increase, at least about 55-fold increase, at least about 56-fold increase, at least about 57-fold increase, at least about 58-fold increase, at least about 59 fold-increase, at least about 60-fold increase, at least about 100-fold increase, at least about 200-fold increase, at least about 300-fold increase, at least about 1500-fold increase, 1600-fold increase, at least about 1700-fold increase, at least about 1800-fold increase, at least about 1900-fold increase, at least about 2000-fold increase, 2100-fold increase, 2200-fold increase, at least about 2300-fold increase, at least about 2400-fold increase, at least about 2500-fold increase, at least about 2600-fold increase, 2700-fold increase, at least about 2800-fold increase, at least about 2900-fold increase, at least about 3000-fold increase, at least about 150000-fold increase, 160000-fold increase, at least about 170000-fold increase, at least about 180000-fold increase, at least about 190000-fold increase, at least about 200000-fold increase, 210000-fold increase, 220000-fold increase, at least about 230000-fold increase, at least about 240000-fold increase, at least about 250000-fold increase, at least about 260000-fold increase, 270000-fold increase, at least about 280000-fold increase, at least about 290000-fold increase or at least about 300000-fold increase in the expression of GM-CSF (e.g. a fold increase in an amount of GM-CSF mRNA after normalization by GAPDH and standardization to a control sample about 3 or 4 days post transfection/transduction, as measured by qRT-PCR analysis).

In some examples, the gRNA is capable of guiding a dCas9 fusion protein/CRISPR/dCas9 synergistic activation mediators (CRISPR/dCas9-SAM) complex/dCas9 ribonucleoprotein complex (e.g. a complex comprising the dCas9 fusion protein) to a target site/target genomic locus that is/that is in proximity of the promoter region of G-CSF, and inducing at least about 0.1-fold increase, at least about 0.2-fold increase, at least about 0.3-fold increase, at least about 0.4-fold increase, at least about 0.5-fold increase, at least about 0.6-fold increase, at least about 0.7-fold increase, at least about 0.8-fold increase, at least about 0.9-fold increase, at least about 1-fold increase, at least about 1.1-fold increase, at least about 1.2-fold increase, at least about 1.3-fold increase, at least about 1.4-fold increase, at least about 1.5-fold increase, at least 1.6-fold increase, at least 1.7-fold increase, at least 1.8-fold increase, at least 1.9-fold increase, at least about 2-fold increase, at least about 2.1-fold increase, at least about 2.2-fold increase, at least about 2.3-fold increase, at least about 2.4-fold increase, at least about 2.5-fold increase, at least about 2.6-fold increase, at least about 2.7-fold increase, at least about 2.8-fold increase, at least about 2.9-fold increase, at least about 3-fold increase, at least about 3.5-fold increase, at least about 4-fold increase, at least about 4.5-fold increase, at least about 5-fold increase, at least about 10-fold increase, at least about 15-fold increase, at least about 20-fold increase, at least about 25-fold increase, at least about 30-fold increase, at least about 31-fold increase, at least about 32-fold increase, at least about 33-fold increase, at least about 34-fold increase, at least about 35-fold increase, at least about 36-fold increase, at least about 37-fold increase, at least about 38-fold increase, at least about 39 fold-increase, at least about 40-fold increase, at least about 41-fold increase, at least about 42-fold increase, at least about 43-fold increase, at least about 44-fold increase, at least about 45-fold increase, at least about 46-fold increase, at least about 47-fold increase, at least about 48-fold increase, at least about 49 fold-increase, at least about 50-fold increase, at least about 51-fold increase, at least about 52-fold increase, at least about 53-fold increase, at least about 54-fold increase, at least about 55-fold increase, at least about 56-fold increase, at least about 57-fold increase, at least about 58-fold increase, at least about 59 fold-increase, at least about 60-fold increase, at least about 100-fold increase, at least about 200-fold increase, at least about 300-fold increase, at least about 1500-fold increase, 1600-fold increase, at least about 1700-fold increase, at least about 1800-fold increase, at least about 1900-fold increase, at least about 2000-fold increase, 2100-fold increase, 2200-fold increase, at least about 2300-fold increase, at least about 2400-fold increase, at least about 2500-fold increase, at least about 2600-fold increase, 2700-fold increase, at least about 2800-fold increase, at least about 2900-fold increase, at least about 3000-fold increase, at least about 150000-fold increase, 160000-fold increase, at least about 170000-fold increase, at least about 180000-fold increase, at least about 190000-fold increase, at least about 200000-fold increase, 210000-fold increase, 220000-fold increase, at least about 230000-fold increase, at least about 240000-fold increase, at least about 250000-fold increase, at least about 260000-fold increase, 270000-fold increase, at least about 280000-fold increase, at least about 290000-fold increase or at least about 300000-fold increase in the expression of G-CSF (e.g. a fold increase in an amount of G-CSF mRNA after normalization by GAPDH and standardization to a control sample about 3 or 4 days post transfection/transduction, as measured by qRT-PCR analysis).

In some examples, the gRNA has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least 100% identity with a sequence selected from Table 1 or Table 4 below:

TABLE 1

| | | | | | SEQ |
|---|---|---|---|---|---|
| | | | | | ID |
| Name | Position | Strand | Sequence | PAM | NO. |
| PAX6_4 | 31811163 | 1 | AATGTGTGTGTGCCGGCGCC | CGG | 1 |
| PAX6_3 | 31811122 | 1 | GCCAGCACACCTATGCTGAT | TGG | 2 |
| PAX6_5 | 31811191 | −1 | GCTTCGCTAATGGGCCAGTG | AGG | 3 |
| PAX6_1 | 31811054 | 1 | ACAATAAAATGGGCTGTCAG | CGG | 4 |
| PAX6_2 | 31811082 | 1 | GAGTGAGAGATAAAGAGTGT | GGG | 5 |
| MITF_1 | 69739390 | 1 | CGGGCCGAACTACAGATCCC | AGG | 6 |
| MITF_2 | 69739276 | 1 | CCAAACAGGAGTTGCACTAG | CGG | 7 |
| MITF_4 | 69739338 | 1 | AGCTGTAGTTTTCGTGGGAG | CGG | 8 |
| MITF_3 | 69739291 | −1 | GCGGGGGAGAGGCAACGTGG | TGG | 9 |
| MITF_5 | 69739214 | 1 | CTGTACCCTTGAAGCAAGTG | GGG | 10 |
| OTX2_2 | 56810650 | −1 | GAACATTCTGGTAATGTCGG | AGG | 11 |
| OTX2_1 | 56810495 | −1 | GCGTCAAAAAGTTGCCAGAG | AGG | 12 |
| OTX2_4 | 56810615 | 1 | AACAGGCCGCTGCTGCACGG | GGG | 13 |
| OTX2_5 | 56810559 | 1 | GATTGACACATCTAAGCCAG | AGG | 14 |
| OTX2_3 | 56810590 | 1 | TAAAAACACACAACAGGGGG | AGG | 15 |

TABLE 4
sgRNA sequence for:

| | | | | SEQ |
|---|---|---|---|---|
| Gene | TSS | | Top/ | ID |
| Name | Distance | Guide Sequence | Bottom | NO: |
| EPO_g1 | 35 | GGGGTGGCCCAGGGACTCTG | b | 76 |
| EPO_g2 | 126 | TGTGCGTGAGGGGTCGCCAG | b | 77 |
| EPO_g3 | 158 | GCCCCTGCTCTGACCCCGGG | t | 78 |
| EPO_g4 | 116 | GGAGAGGCTGTGTGCGTGAG | b | 79 |
| SCF_g1 | 25 | GAACTGTATAAAAGCGCCGG | t | 80 |
| SCF_g2 | 159 | CCTAATCTGCCAAACTTCTG | t | 81 |
| SCF_g3 | 51 | GAGGCGTGTCCGGAGCAGGC | t | 82 |
| SCF_g4 | 90 | GGTAGGCGAGAAGCAGGCAA | b | 83 |
| SCF_g5 | 69 | TCCTTCCCTTCCGGAGCCCG | b | 84 |
| TPO_g1 | 79 | GAGCCACCAGACACTGGTGA | b | 85 |
| TPO_g2 | 198 | CCCTATCCAAATCTTCTCCG | t | 86 |
| TPO_g3 | 57 | ACTTCTGCCCAATCAGAGAA | b | 87 |
| TPO_g4 | 106 | AAGAGAAGGCGTCACTTCCG | t | 88 |
| TPO_g5 | 32 | AGCAGGTCATACGCCTGCCT | b | 89 |
| GM- | 16 | AAGAGCTCTTAAATACACAG | b | 90 |
| CSF_g1 | | | | |
| GM- | 50 | GTGACCACAAAATGCCAGGG | b | 91 |
| CSF_g2 | | | | |
| GM- | 73 | CGGGGGAACTACCTGAACTG | b | 92 |
| CSF_g3 | | | | |
| GM- | 104 | GGCCCTTATCAGCCACACAT | b | 93 |
| CSF_g4 | | | | |
| GM- | 117 | AGGCTCACCGTTCCCATGTG | t | 94 |
| CSF_g5 | | | | |
| G-CSF_g1 | 39 | GTGTCCAAGACAATGCAGGG | b | 95 |
| G-CSF_g2 | 116 | GGGCAAGGCGACGTCAAAGG | t | 96 |
| G-CSF_g3 | 80 | GCGAAAGTTTTGTGAAATTG | b | 97 |
| G-CSF_g4 | 120 | GGGGGGCAAGGCGACGTCAA | t | 98 |
| G-CSF_g5 | 22 | CACCAAATTTGCATAAATCC | b | 99 |

In various embodiments, the gRNA has at least about 80% identity with a sequence selected the group consisting of SEQ ID NO: 1 (AATGTGTGTGTGCCGGCGCC), SEQ ID NO: 2 (GCCAGCACACCTATGCTGAT), SEQ ID NO: 3 (GCTTCGCTAATGGGCCAGTG), SEQ ID NO: 4 (ACAATAAAATGGGCTGTCAG), SEQ ID NO: 5 (GAGTGAGAGATAAAGAGTGT), SEQ ID NO: 6 (CGGGCCGAACTACAGATCCC), SEQ ID NO: 7 (CCAAACAGGAGTTGCACTAG), SEQ ID NO: 8 (AGCTGTAGTTTTCGTGGGAG), SEQ ID NO: 9 (GCGGGGGAGAGGCAACGTGG), SEQ ID NO: 10 (CTGTACCCTTGAAGCAAGTG), SEQ ID NO: 11 (GAACATTCTGGTAATGTCGG), SEQ ID NO: 12 (GCGTCAAAAAGTTGCCAGAG), SEQ ID NO: 13 (AACAGGCCGCTGCTGCACGG), SEQ ID NO: 14 (GATTGACACATCTAAGCCAG), SEQ ID NO: 15 (TAAAAACACACAACAGGGGG), SEQ ID NO: 76 (GGGGTGGCCCAGGGACTCTG), SEQ ID NO: 77 (TGTGCGTGAGGGGTCGCCAG), SEQ ID NO: 78 (GCCCCTGCTCTGACCCCGGG), SEQ ID NO: 79 (GGAGAGGCTGTGTGCGTGAG), SEQ ID NO: 80 (GAACTGTATAAAAGCGCCGG), SEQ ID NO: 81 (CCTAATCTGCCAAACTTCTG), SEQ ID NO: 82 (GAGGCGTGTCCGGAGCAGGC), SEQ ID NO: 83 (GGTAGGCGAGAAGCAGGCAA), SEQ ID NO: 84 (TCCTTCCCTTCCGGAGCCCG), SEQ ID NO: 85 (GAGCCACCAGACACTGGTGA), SEQ ID NO: 86 (CCCTATCCAAATCTTCTCCG), SEQ ID NO: 87 (ACTTCTGCCCAATCAGAGAA), SEQ ID NO: 88 (AAGAGAAGGCGTCACTTCCG), SEQ ID NO: 89 (AGCAGGTCATACGCCTGCCT), SEQ ID NO: 90 (AAGAGCTCTTAAATACACAG), SEQ ID NO: 91 (GTGACCACAAAATGCCAGGG), SEQ ID NO: 92 (CGGGGGAACTACCTGAACTG), SEQ ID NO: 93 (GGCCCTTATCAGCCACACAT), SEQ ID NO: 94 (AGGCTCACCGTTCCCATGTG), SEQ ID NO: 95 (GTGTCCAAGACAATGCAGGG), SEQ ID NO: 96 (GGGCAAGGCGACGTCAAAGG), SEQ ID NO: 97 (GCGAAAGTTTTGTGAAATTG), SEQ ID NO: 98 (GGGGGGCAAGGCGACGTCAA), and SEQ ID NO: 99 (CACCAAATTTGCATAAATCC).

In various embodiments, the gRNA has about 15 bp to about 25 bp. In some examples, the gRNA has about 20 bp.

In various embodiments, the gRNA is a single/short gRNA (sgRNA). As used herein, a “single or short guide RNA (sgRNA)” refers to single guide RNA used in conjunction with CRISPR associated systems (Cas). sgRNAs are a fusion of crRNA and tracrRNA and may contain nucleotides of sequences complementary to the desired target site.

Also disclosed is a set of gRNA comprising at least two of the gRNA as described herein. In various examples, the set of gRNA may include, but is not limited to, a gRNA that is specific to a target site that is/that is in proximity of the promoter region of PAX6, a gRNA that is specific to a target site that is/that is in proximity of the promoter region of MITF and a gRNA that is specific to a target site that is/that is in proximity of the promoter region of OTX2.

In some examples, the set of gRNA as described herein, when bound/associated with one or more dCas9 fusion protein/CRISPR/dCas9 synergistic activation mediators (CRISPR/dCas9-SAM) complex/dCas9 ribonucleoprotein complex (e.g. a complex comprising the dCas9 fusion protein), is capable of inducing at least about 40-fold change, at least about 45-foldchange, at least about 50-fold change, at least about 55-fold change, at least about 60-fold change, at least about 65-fold change, at least about 70-fold change, at least about 75-fold change or at least about 80-fold change in the expression of PAX6 and MITF (e.g. a fold increase in an amount of PAX6 and MITF mRNA about 4 days post transfection/transduction, after normalization between 0 to 100 with GAPDH and standardization with a control sample) in a cell, optionally wherein the set of gRNA is capable of inducing substantially similar fold changes in the expression of PAX6 and MITF.

In some examples, the set of gRNA as described herein, when bound/associated with one or more dCas9 fusion protein/CRISPR/dCas9 synergistic activation mediators (CRISPR/dCas9-SAM) complex/dCas9 ribonucleoprotein complex (e.g. a complex comprising the dCas9 fusion protein), is capable of inducing at least about 40-fold change, at least about 45-foldchange, at least about 50-fold change, at least about 55-fold change, at least about 60-fold change, at least about 65-fold change, at least about 70-fold change, at least about 75-fold change or at least about 80-fold change in the expression of PAX6 and OTX2 (e.g. a fold increase in an amount of PAX6 and OTX2 mRNA about 4 days post transfection/transduction, after normalization between 0 to 100 with GAPDH and standardization with a control sample) in a cell, optionally wherein the set of gRNA is capable of inducing substantially similar fold changes in the expression of PAX6 and OTX2.

In some examples, the set of gRNA as described herein, when bound/associated with one or more dCas9 fusion protein/CRISPR/dCas9 synergistic activation mediators (CRISPR/dCas9-SAM) complex/dCas9 ribonucleoprotein complex (e.g. a complex comprising the dCas9 fusion protein), is capable of inducing at least about 40-fold change, at least about 45 fold-change, at least about 50-fold change, at least about 55-fold change, at least about 60-fold change, at least about 65-fold change, at least about 70-fold change, at least about 75-fold change, at least about 80-fold change, at least about 85-fold change or at least about 90-fold change in the expression of MITF and OTX2 (e.g. a fold increase in an amount of MITF and OTX2 mRNA about 4 days post transfection/transduction, after normalization between 0 to 100 with GAPDH and standardization with a control sample) in a cell, optionally wherein the set of gRNA is capable of inducing substantially similar fold changes in the expression of MITF and OTX2.

In some examples, the set of gRNA as described herein, when bound/associated with one or more dCas9 fusion protein/CRISPR/dCas9 synergistic activation mediators (CRISPR/dCas9-SAM) complex/dCas9 ribonucleoprotein complex (e.g. a complex comprising the dCas9 fusion protein), is capable of inducing at least about 20-fold change, at least about 25 fold-change, at least about 30-fold change, at least about 35-fold change, at least about 40-fold change, at least about 45 fold-change, at least about 50-fold change, at least about 55-fold change, at least about 60-fold change, at least about 65-fold change, at least about 70-fold change, at least about 75-fold change or at least about 80-fold change in the expression of PAX6, MITF and OTX2 (e.g. a fold increase in an amount of PAX6, MITF and OTX2 mRNA about 4 days post transfection/transduction, after normalization between 0 to 100 with GAPDH and standardization with a control sample) in a cell, optionally wherein the set of gRNA is capable of inducing substantially similar fold changes in the expression of PAX6, MITF and OTX2.

In some examples, the set comprises at least two gRNA may include, but is not limited to:

a gRNA at least about 80%, at least about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 99%, at least about 99%, or at least 100% identity with SEQ ID NO: 5;

a gRNA at least about 80%, at least about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 99%, at least about 99%, or at least 100% identity with SEQ ID NO: 9; and

a gRNA at least about 80%, at least about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 99%, at least about 99%, or at least 100% identity with SEQ ID NO: 13.

Also disclosed is an oligonucleotide/primer, optionally an oligonucleotide/primer, for cloning a gRNA of any of the preceding AS, the oligonucleotide/primer having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 99%, at least about 99%, or at least 100% identity with a sequence selected from Table 2.

In another aspect, there is provided an oligonucleotide/primer for cloning a gRNA of any of claims 25 to 30, the oligonucleotide/primer having at least about 80% with a sequence selected from Table 2 below:

TABLE 2

| | | SEQ ID |
|---|---|---|
| Name | Sequence | NO. |
| Pax6_1_Fwd | CACCGACAATAAAATGGGCTGTCAG | 16 |
| Pax6_1_Rev | AAACCTGACAGCCCATTTTATTGTC | 17 |
| Pax6_2_Fwd | CACCGGAGTGAGAGATAAAGAGTGT | 18 |
| Pax6_2_Rev | AAACACACTCTTTATCTCTCACTCC | 19 |
| Pax6_3_Fwd | CACCGGCCAGCACACCTATGCTGAT | 20 |
| Pax6_3_Rev | AAACATCAGCATAGGTGTGCTGGCC | 21 |
| Pax6_4_Fwd | CACCGAATGTGTGTGTGCCGGCGCC | 22 |
| Pax6_4_Rev | AAACGGCGCCGGCACACACACATTC | 23 |
| Pax6_5_Fwd | CACCGGCTTCGCTAATGGGCCAGTG | 24 |
| Pax6_5_Rev | AAACCACTGGCCCATTAGCGAAGCC | 25 |
| MITF_1_Fwd | CACCGCGGGCCGAACTACAGATCCC | 26 |
| MITF_1_Rev | AAACGGGATCTGTAGTTCGGCCCGC | 27 |
| MITF_2_Fwd | CACCGCCAAACAGGAGTTGCACTAG | 28 |
| MITF_2_Rev | AAACCTAGTGCAACTCCTGTTTGGC | 29 |
| MITF_3_Fwd | CACCGGCGGGGGAGAGGCAACGTGG | 30 |
| MITF_3_Rev | AAACCCACGTTGCCTCTCCCCCGCC | 31 |
| MITF_4_Fwd | CACCGAGCTGTAGTTTTCGTGGGAG | 32 |
| MITF_4_Rev | AAACCTCCCACGAAAACTACAGCTC | 33 |
| MITF_5_Fwd | CACCGCTGTACCCTTGAAGCAAGTG | 34 |
| MITF_5_Rev | AAACCACTTGCTTCAAGGGTACAGC | 35 |
| OTX2_1_Fwd | CACCGGCGTCAAAAAGTTGCCAGAG | 36 |
| OTX2_1_Rev | AAACCTCTGGCAACTTTTTGACGCC | 37 |
| OTX2_2_Fwd | CACCGGAACATTCTGGTAATGTCGG | 38 |
| OTX2_2_Rev | AAACCCGACATTACCAGAATGTTCC | 39 |
| OTX2_3_Fwd | CACCGTAAAAACACACAACAGGGGG | 40 |
| OTX2_3_Rev | AAACCCCCCTGTTGTGTGTTTTTAC | 41 |
| OTX2_4_Fwd | CACCGAACAGGCCGCTGCTGCACGG | 42 |
| OTX2_4_Rev | AAACCCGTGCAGCAGCGGCCTGTTC | 43 |
| OTX2_5_Fwd | CACCGGATTGACACATCTAAGCCAG | 44 |
| OTX2_5_Rev | AAACCTGGCTTAGATGTGTCAATCC | 45 |

Also disclosed is an oligonucleotide/primer, optionally an oligonucleotide/primer, for analyzing gene expression of a cell/cell population/sheet of any of the preceding AS, the oligonucleotide/primer having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 99%, at least about 99%, or at least 100% identity with a sequence selected from Table 3 below:

TABLE 3

| | | SEQ | | SEQ |
|---|---|---|---|---|
| | | ID | | ID |
| | Forward | NO. | Reverse | NO. |
| Oct3/4 | CAGTGCCCGAAACCCACAC | 46 | GGAGACCCAGCAGCCTCAAA | 47 |
| Lhx2 | CGTCCGTCTTAACTTCTGTGC | 48 | AGGTTGGTAAGAGTCGTTTGT | 49 |
| Rax | TCCCAGGAGGCTTGGAGACCC | 50 | CTCCCCAAGTCCTGAGCGTGC | 51 |
| Otx2 | AGTTCCGAGAGCCATAGAAGG | 52 | TAAGCAGATTGGTTTGTCCAT | 53 |
| Pax6(+5a) | CTCGGTGGTGTCTTTGTCAAC | 54 | ACTTTTGCATCTGCATGGGTC | 55 |
| Mitf | CCCAGTTCATGCAACAGAGAG | 56 | GCAGAGGGAAGGGTGGTG | 57 |
| Tyrosinase | GTGTAGCCTTCTTCCAACTCAG | 58 | GTTCCTCATTACCAAATAGCATCC | 59 |
| Tyrp1 | GATTCCACTCTAATAAGCCCAAA | 60 | TTCCAAGCACTGAGCGACAT | 61 |
| Tyrp2 | CTCAGACCAACTTGGCTACAGCTA | 62 | CAGCACAAAAAGACCAACCAAA | 63 |
| pmel17 | TGATGGCTGTGGTCCTTGC | 64 | CAGTGACTGCTGCTATGTGG | 65 |
| PEDF | TATCACCTTAACCAGCCTTTCATC | 66 | GGGTCCAGAATCTTGCCAATG | 67 |
| Best1 | TAGAACCATCAGCGCCGTC | 68 | TGAGTGTAGTGTGTATGTTGG | 69 |
| Cralbp | CACGCTGCCCAAGTATGATG | 70 | CCAGGACAGTTGAGGAGAGG | 71 |
| RPE65 | CCTGATTCATACCCATCAGAACCC | 72 | CACCACACTCAGAACTACACCATC | 73 |
| GAPDH | AGCAAGAGCACAAGAGGAAGAG | 74 | GAGCACAGGGTACTTTATTGATGG | 75 |

Also disclosed is a composition comprising: a dCas9 fusion protein, the dCas9 fusion protein comprising dCas9 and an effector; a gRNA, optionally a sgRNA, wherein the gRNA is capable of guiding the dCas9 fusion protein to a target site that is/that is in proximity of the promoter region of one or more differentiation factors to allow the dCas9 fusion protein to modulate the expression of the one or more differentiation factors; and optionally an activator module comprising a RNA-binding protein capable of binding to the gRNA, further optionally wherein the RNA-binding protein comprises MS2 coat protein (MCP).

Also disclosed is a composition/system comprising: a CRISPR/dCas9 synergistic activation mediators (CRISPR/dCas9-SAM) complex/dCas9 ribonucleoprotein complex (e.g. a complex comprising the dCas9 fusion protein)/dCas9-VP64/dCas9-VPR/dCas9-VP64 and MS2-P65-HSF1; and a gRNA, optionally sgRNA, wherein the gRNA is capable of guiding the dCas9 fusion protein to a target site that is/that is in proximity of the promoter region of one or more differentiation factors e.g. a differentiation factor that influences cell differentiation, cell dedifferentiation, cell reprogramming and/or cell transdifferentiation e.g. to allow the dCas9 fusion protein to modulate the expression of the one or more differentiation factors.

Also disclosed is a kit comprising for altering a differentiation status of a cell, the method comprising: a nucleic acid transcribing a gRNA, optionally a sgRNA, that is capable of guiding a dCas9 fusion protein to a target site that is/that is in proximity of the promoter region of the one or more differentiation factors e.g. a differentiation factor that influences cell differentiation, cell dedifferentiation, cell reprogramming and/or cell transdifferentiation e.g. to allow the dCas9 fusion protein to modulate the expression of the one or more differentiation factors.

In another aspect, there is provided a kit comprising reagents for altering a differentiation status of a cell, the kit comprising: a nucleic acid transcribing a gRNA, optionally a sgRNA, that is capable of guiding a dCas9 fusion protein to a target site that is/that is in proximity of the promoter region of the one or more differentiation factors to allow the dCas9 fusion protein to modulate the expression of the one or more differentiation factors.

In various embodiments, the kit further comprising one or more of the following:

a) a second or further nucleic acid transcribing a second or further gRNA, optionally a sgRNA, that is capable of guiding a dCas9 fusion protein to a target site that is/that is in proximity of the promoter region of the one or more differentiation factors e.g. a differentiation factor that influences cell differentiation, cell dedifferentiation, cell reprogramming and/or cell transdifferentiation e.g. to allow the dCas9 fusion protein to modulate the expression of the one or more differentiation factors;

b) a nucleic acid encoding a dCas9 fusion protein, optionally a dCas9-VP64 fusion protein and/or a dCas9-VPR fusion protein;

c) a nucleic acid encoding an activator module comprising a RNA-binding protein capable of binding to the gRNA, optionally wherein the RNA-binding protein comprises MS2 coat protein (MCP), further optionally wherein the nucleic acid encodes MCP, HSF1 and/or p65;

d) CRISPR/dCas9 synergistic activation mediators (CRISPR/dCas9-SAM) complex/dCas9 ribonucleoprotein complex (e.g. a complex comprising the dCas9 fusion protein)/dCas9-VP64/dCas9-VPR/dCas9-VP64 and MS2-P65-HSF1;

e) one or more oligonucleotide/primer having at least about 80%, at least about at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 99%, at least about 99%, or at least 100% identity with a sequence selected from Tables 2 and 3;

f) a viral vector, a virus packaging plasmid and/or a virus expression vector;

g) a nucleic acid construct/expression construct/expression vector/plasmid/viral vector/recombinant construct as described herein;

h) one or more probes, capture agents, dyes, labels, nucleotides, salts, buffering agents, various additives, PCR enhancers and combinations thereof; and

i) instructions for use.

Also disclosed is a method of treating a disease, the method comprising transplanting the RPE cell/RPE cell line/RPE cell population/RPE sheet as described herein to a patient in need thereof. In one aspect, there is provided a method of treating a disease, the method comprising transplanting the differentiated/altered cell as described herein to a patient in need thereof.

In various embodiments, the disease is an eye disease/disorder, optionally wherein the eye disease/disorder is selected from the group consisting of macular degeneration, acute macular degeneration (AMD), atrophic age-related macular degeneration (atrophic AMD), dry age-related macular degeneration (Dry-type AMD), retinitis pigmentosa (RP), Stargardt's disease, and myopia.

Also disclosed in the cell/cell line/stem cell/human cell/specialized cell/engineered cell/RPE cell/RPE cell line/RPE cell population/RPE sheet, the nucleic acid construct/expression construct/expression vector/plasmid/viral vector/recombinant construct, the gRNA, the set of gRNA, the oligonucleotide/primer, the composition/system or the kit as described herein for use in stem cell therapy, regenerative medicine, reversing vision loss and/or treating eye/retinal diseases.

Also disclosed is a method, a product, a cell/cell line/stem cell/human cell/specialized cell/engineered cell/RPE cell/RPE cell line/RPE cell population/RPE sheet, a nucleic acid construct/expression construct/expression vector/plasmid/viral vector/recombinant construct, a gRNA, a set of gRNA, a oligonucleotide/primer, a composition/system or a kit as described herein.

Also disclosed is a cell/cell line/human cell/specialized cell/engineered cell/RPE cell/RPE cell line/RPE cell population/RPE sheet produced by the method as described herein, wherein the cell/cell line/human cell/specialized cell/engineered cell/RPE cell/RPE cell line/RPE cell population/RPE sheet at about Day 28 (of CRISPR/dCas9-SAM activated differentiation) comprises one or more of the following characteristics as compared to a commercial human RPE cell/cell population (from Lonza) at about Day 21:
a) substantially similar expression (mRNA level) of one or more genes selected from the group consisting of: Oct4-, OTX2, μMEL17, RPE65, LHX2 and RAX;
b) increased expression (mRNA level) of one or more genes selected from the group consisting of: Pax6, MITF and Tyrp2; and
c) decreased/reduced expression (mRNA level) of one or more genes selected from the group consisting of: Tyr, Tyrp1, CRALBP, BEST1 and PEDF.

Also disclosed is a method, a product, a cell/cell line/stem cell/human cell/specialized cell/engineered cell/RPE cell/RPE cell line/RPE cell population/RPE sheet, a nucleic acid construct/expression construct/expression vector/plasmid/viral vector/recombinant construct, a gRNA, a set of gRNA, a oligonucleotide/primer, a composition/system or a kit as described herein, wherein dCas12a is used in place of dCas9.

In some examples, the method, the product, the cell/cell line/stem cell/human cell/specialized cell/engineered cell/RPE cell/RPE cell line/RPE cell population/RPE sheet, the nucleic acid construct/expression construct/expression vector/plasmid/viral vector/recombinant construct, the gRNA, the set of gRNA, the oligonucleotide/primer, the composition/system or the kit as described herein, wherein the dCas12a is/is derived from/is modified from a Cas12a protein selected from the group consisting of: Acidaminococcus sp. (AsCpf1) Cas12a and Lachnospiraceae bacterium (LbCpf1) Cas12a.

In some examples, the method, the product, the cell/cell line/stem cell/human cell/specialized cell/engineered cell/RPE cell/RPE cell line/RPE cell population/RPE sheet, the nucleic acid construct/expression construct/expression vector/plasmid/viral vector/recombinant construct, the gRNA, the set of gRNA, the oligonucleotide/primer, the composition/system or the kit as described herein, wherein the target site/target genomic locus of dCas12a/dCas12a fusion protein precede, optionally immediately precede, a 5′-TTTN protospacer adjacent motif, wherein N=A or G or T or C.

Also disclosed is a method of altering a cell e.g. a gene expression of the cell, the method comprising: modulating the expression of/activating one or more growth factors/cytokines, optionally extracellular growth factors/cytokines, in the cell with a dCas9/dCas12a fusion protein, the dCas9/dCas12a fusion protein comprising dCas9/dCas12a and an effector.

Also disclosed is a method of altering a cell e.g. a gene expression of the cell, the method comprising: modulating the expression of/activating one or more matrix proteins in the cell with a dCas9/dCas12a fusion protein, the dCas9/dCas12a fusion protein comprising dCas9/dCas12a and an effector.

Also disclosed is a method of producing viral vectors, the method comprising: modulating the expression of/activating one or more viral genes in a host cell with a dCas9/dCas12a fusion protein, the dCas9/dCas12a fusion protein comprising dCas9/dCas12a and an effector.

Also disclosed is a method of altering a cell e.g. a gene expression of the cell, the method comprising: modulating the expression of/activating one or more non-transcription factors in the cell with a dCas9/dCas12a fusion protein, the dCas9/dCas12a fusion protein comprising dCas9/dCas12a and an effector, optionally wherein the cell comprises a beta cell and further optionally wherein the one or more non-transcription factors comprises insulin.

In some examples, the method further comprising one or more of the features described hereinbefore.

Also disclosed is a cell/cell line/cell population/altered cell/viral vector produced by the method as described herein, optionally wherein the cell has a higher expression of the one or more growth factors/cytokines or one or more matrix proteins as compared to a reference/control/wild-type cell.

The term “micro” as used herein is to be interpreted broadly to include dimensions from about 1 micron to about 1000 microns.

The term “nano” as used herein is to be interpreted broadly to include dimensions less than about 1000 nm.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications related to structural, electrical and optical changes may be made without deviating from the scope of the invention. Example embodiments are not necessarily mutually exclusive as some may be combined with one or more embodiments to form new exemplary embodiments.

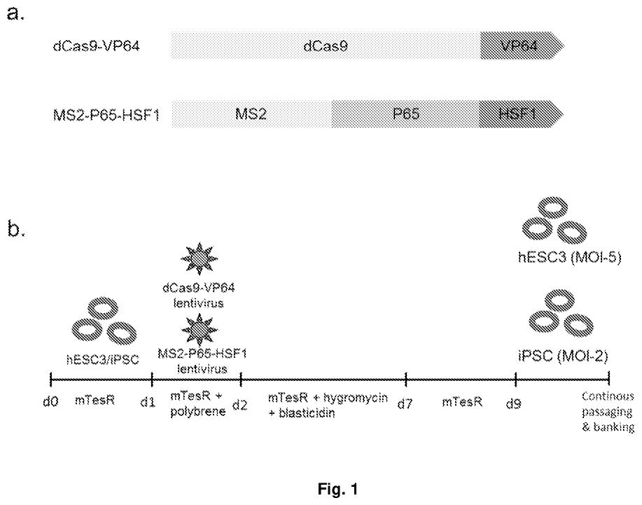

FIG. 1. CRISPR/dCas9-SAM expressing stable cell generation. (a) Schematic representation of CRISPR/dCas9-SAM structures. (b) Schematic diagram of the experimental procedure used for generating CRISPR/dCas9-SAM expressing stable human pluripotent cells.

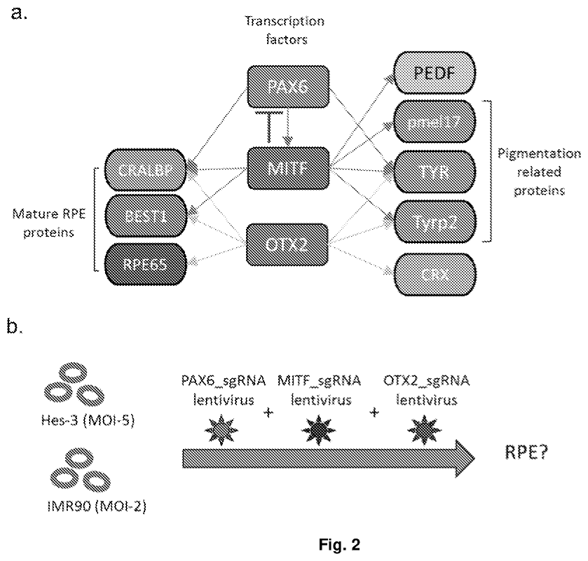

FIG. 2. Directed differentiation approach from pluripotent stem cells to RPEs. (a) Schematic diagram of RPE transcription factors activation network of important RPE specific genes. (b) Schematic diagram of the hypothesis of the present disclosure, by delivering guide RNA sequences specific to activate the three main transcription factors (PAX6, MITF and OTX2) simultaneously in pluripotent stem cells expressing CRISPR/dCas9-SAM may differentiate directly into RPE cells.

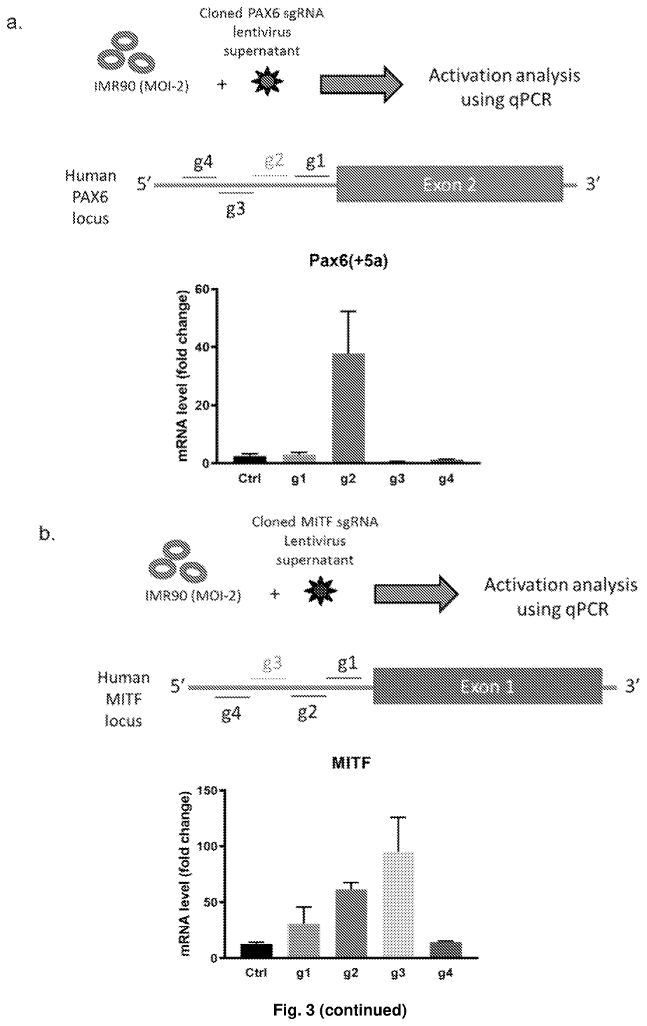

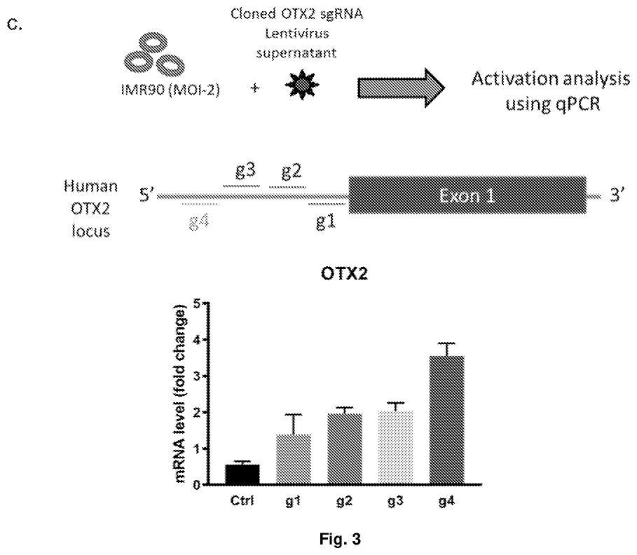

FIG. 3. Functional screening of designed guide RNAs to endogenously activate (a) PAX6, (b) MITF and (c) OTX2 in CRISPR/dCas9-SAM pluripotent stem cells. CRISPR/dCas9-SAM pluripotent stem cells were transduced with indicated sgRNA lentivirus supernatants for each gene of interest. qRT-PCR analysis of mRNA expression levels were measured 4 days post transduction. The mRNA expression levels were normalized by GAPDH and then standardized to that in the sample of sgControl. Values shown are the mean±SE of n=3.

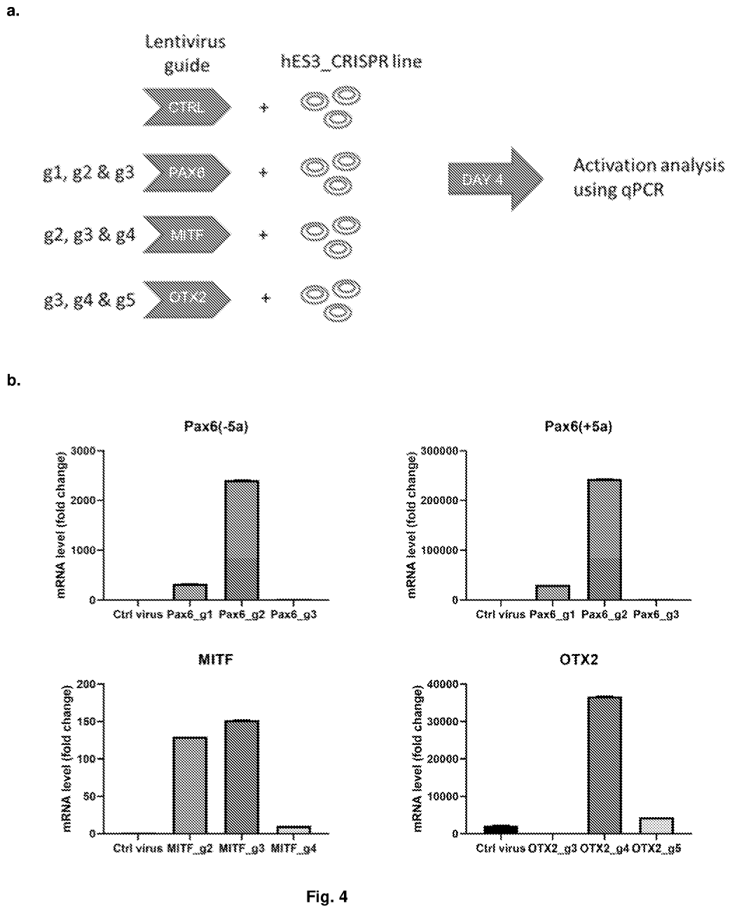

FIG. 4. Endogenous gene activation by using concentrated lentivirus supernatants individually in CRISPR/dCas9-SAM pluripotent stem cells. (a) Schematic diagram of the transduction procedure using concentrated individual top three guides based on our previous gRNA screening. (b) qRT-PCR analysis of mRNA expression levels were measured 4 days post transduction. The mRNA expression levels were normalized by GAPDH and then standardized to that in the sample of sgControl. Values shown are the mean±SE of n=3.

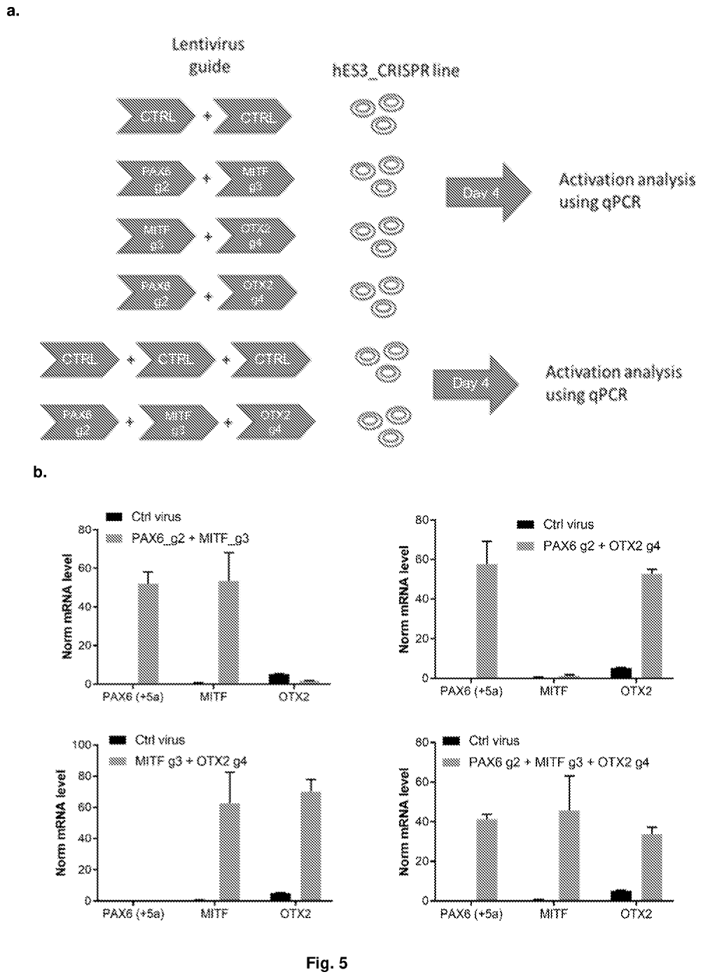

FIG. 5. Multiplex gene activation using concentrated lentivirus supernatants in CRISPR/dCas9-SAM pluripotent stem cells. (a) Schematic diagram of the transduction procedure for multiplex gene activation using concentrated top guide RNA for each gene. (b) qRT-PCR analysis of mRNA expression levels were measured 4 days post transduction. The mRNA expression levels were normalized by GAPDH and then standardized to that in the sample of sgControl. Values shown are the mean±SE of n=3.

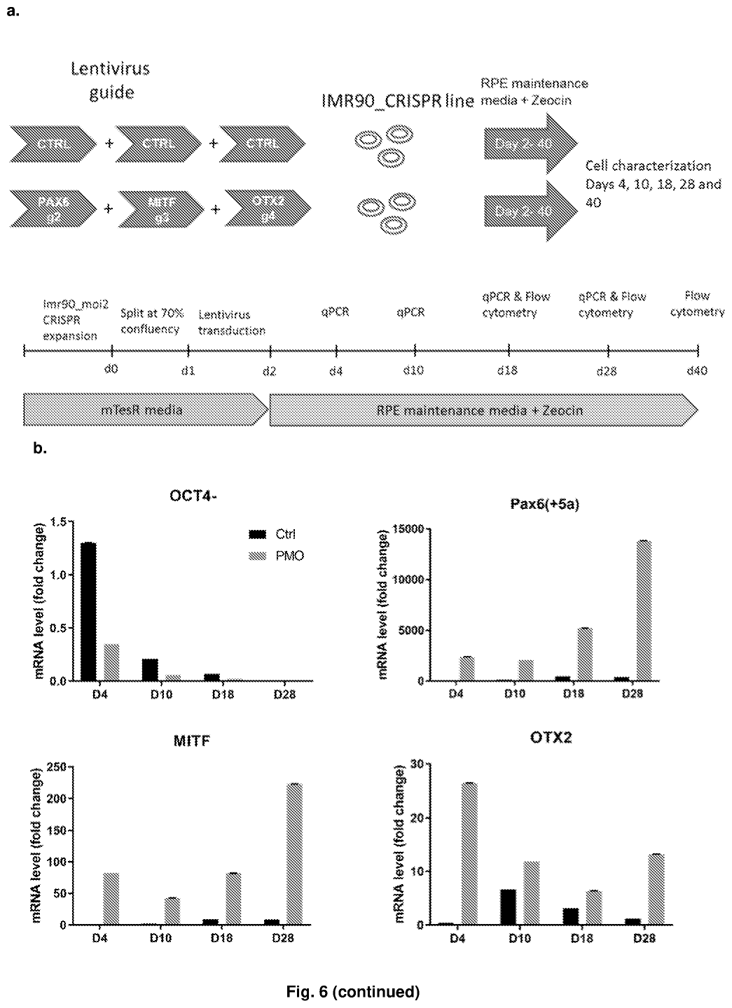

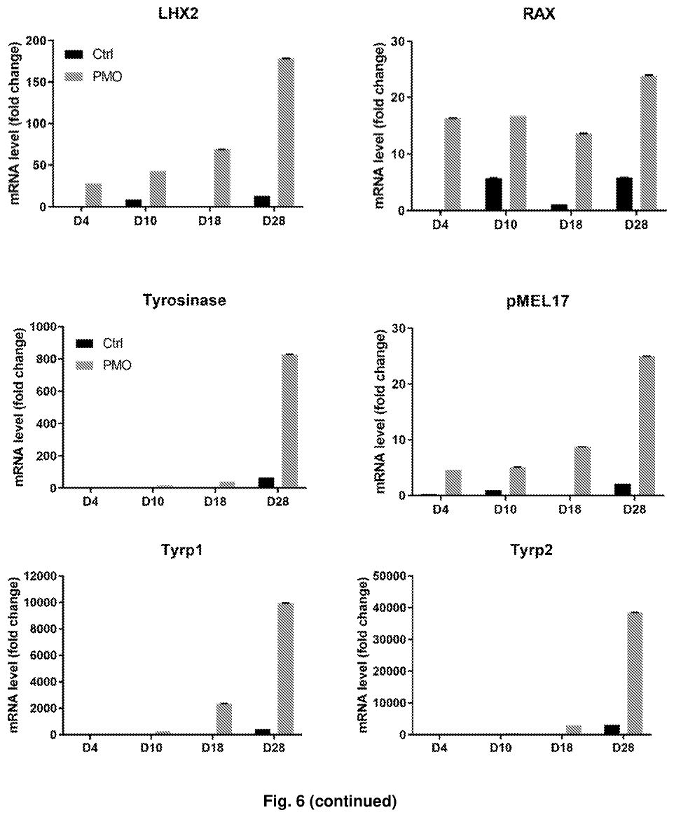

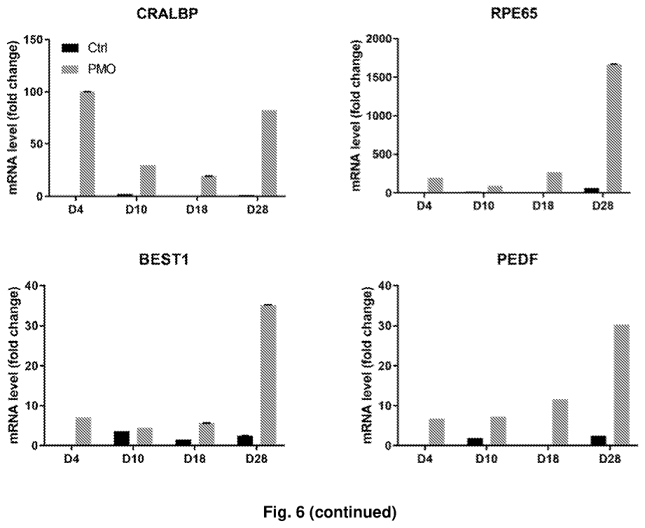

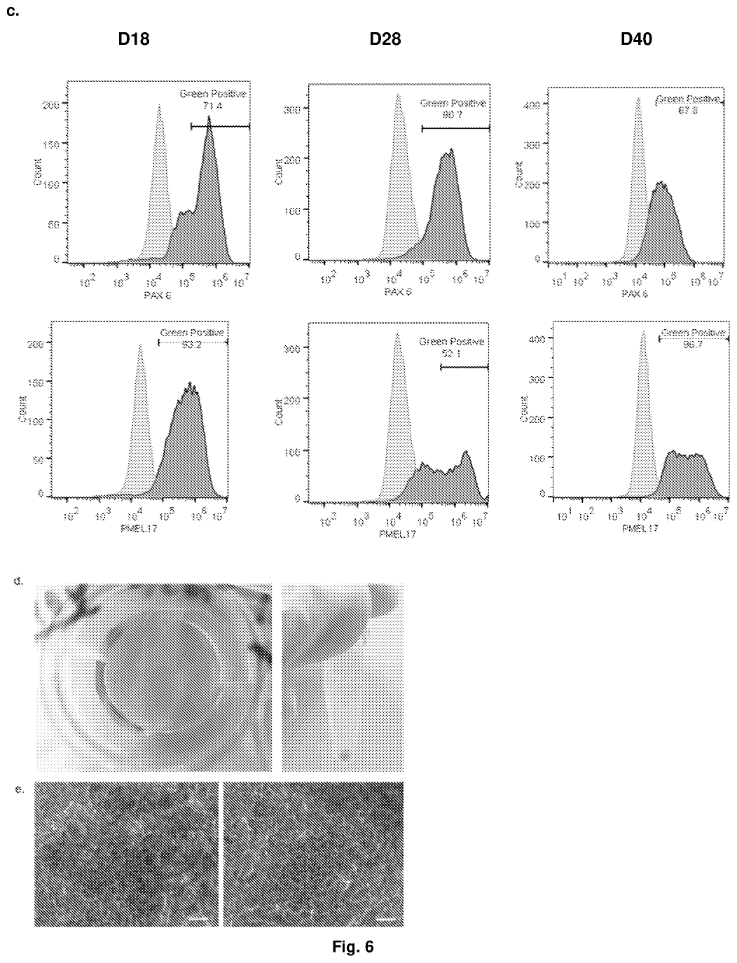

FIG. 6. hiPSC RPE differentiation using CRISPR/dCas9-SAM mediated multiplex endogenous gene activation. (a) Schematic of hiPSC differentiation into RPE using CRISPR/dCas9-mediated multiplex gene activation followed by culture in RPEM. (b) qRT-PCR analysis of pluripotency (Oct4), activated transcription factors (PAX6 (+5a), MITF and OTX2), early eye field genes (LHX2 and RAX), RPE specific pigmentation genes (Tyrosinase, pMEL17, TYRP1 and TYRP2) and RPE specific mature genes (CRALBP, RPE65, BEST1 and PEDF). (c) Flow cytometry histograms of PAX6 and pMEL17 at day 18, 24 and 40. (d & e) Morphology of differentiating hiPSC at day40 in RPEM and RPE cells appear as distinct pigmented foci, with (d) showing macroscopic and (b) showing microscopic images. (Scale bar: 200 μm)

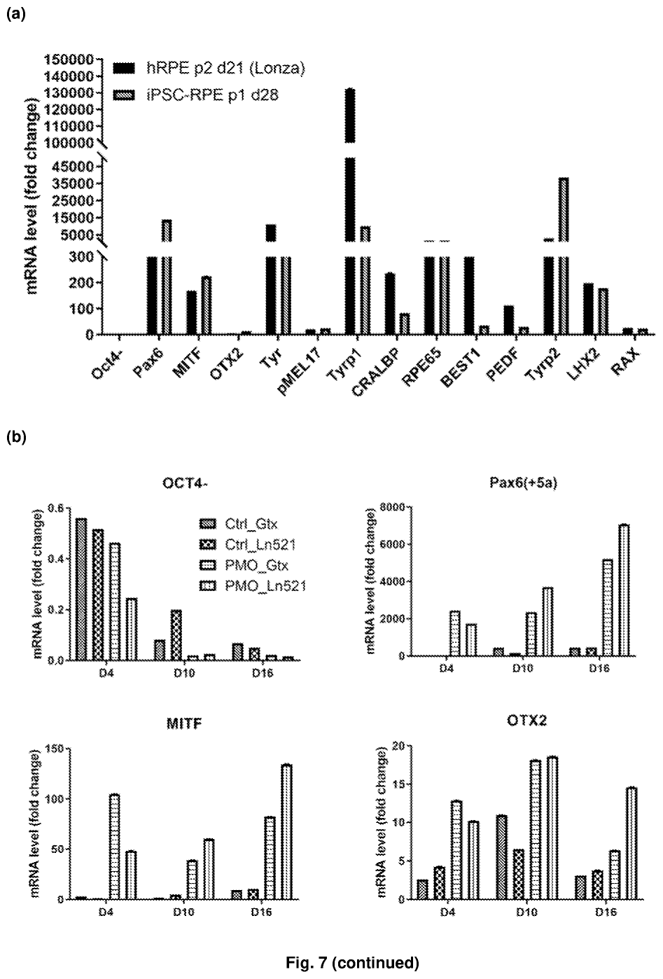

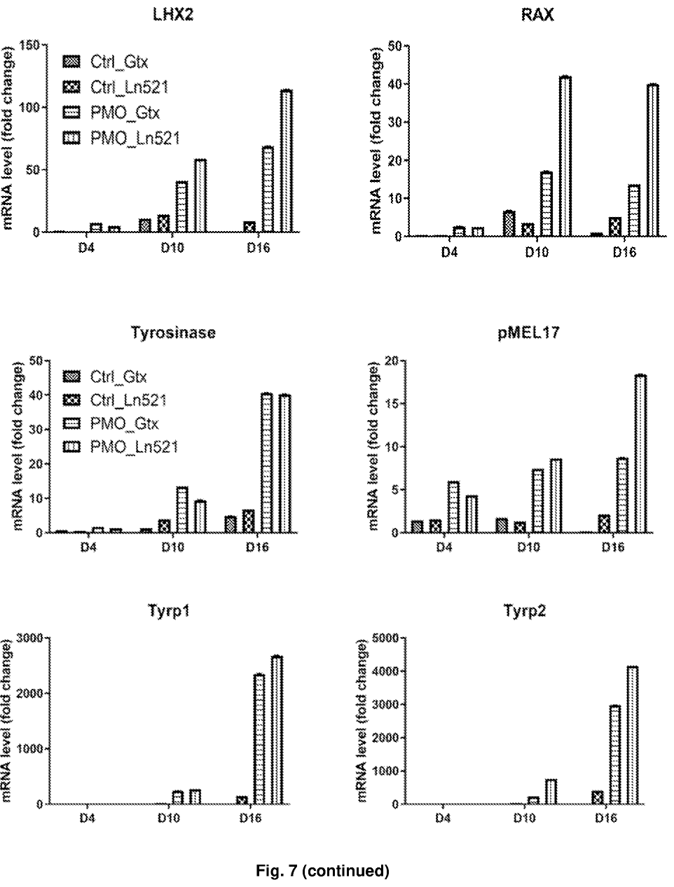

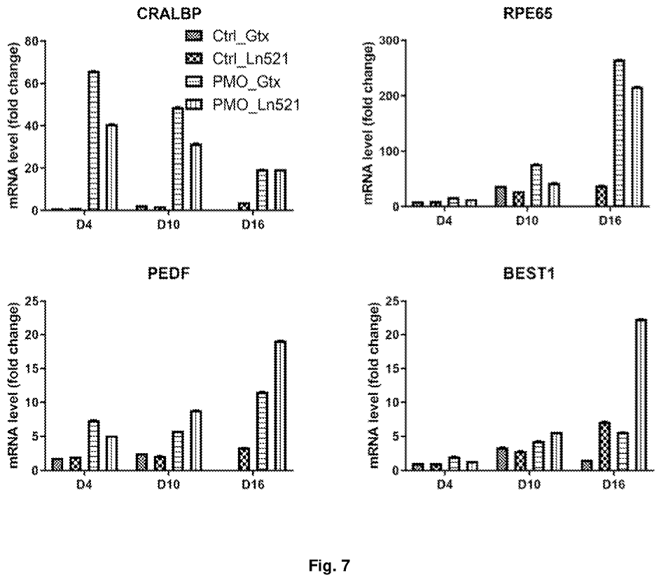

FIG. 7 Characterization of cells during hiPSC RPE differentiation using CRISPR/dCas9-SAM mediated multiplex endogenous gene activation. (a) Comparison of RPE specific gene expression from hRPE (Lonza) and day 28 of hiPSC-CRISPR/dCas9-SAM activated RPE differentiation. (b) Comparison of hiPSC-CRISPR/dCas9-SAM activated RPE differentiation on Matrigel (Gtx) and Laminin 521 (Ln521) following days 4, 10 and 16 of gene activation

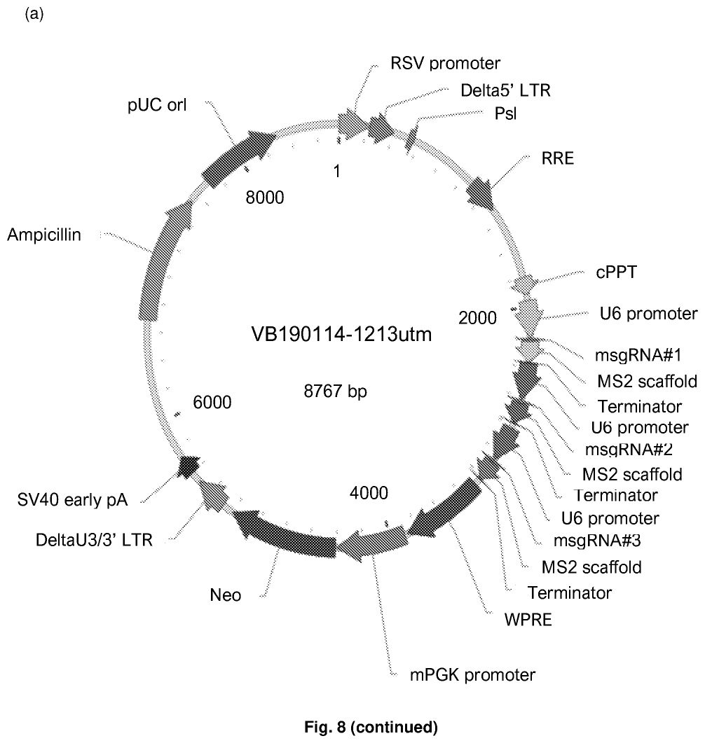

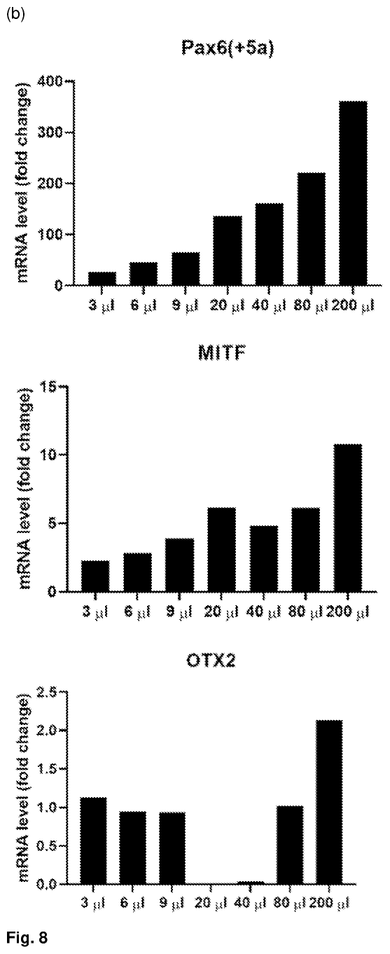

FIG. 8. Lentiviral RPE triple sgRNA vector design and characterization. (a) Lentiviral plasmid encoded with PAX6, MITF and OTX2 sgRNA sequences. Each sgRNA with MS2 scaffold is placed under the control of U6 promoter followed by a terminator sequence. (b) Gene expression data from iPSC CRISPR-SAM cells transduced with triple guide lentivirus at different concentration. The error bars represent the standard error of the mean. Abbreviations: PAX6 (Paired box protein), OTX2 (Orthodenticle homeobox 2), MITF (Melanocyte Inducing Transcription Factor).

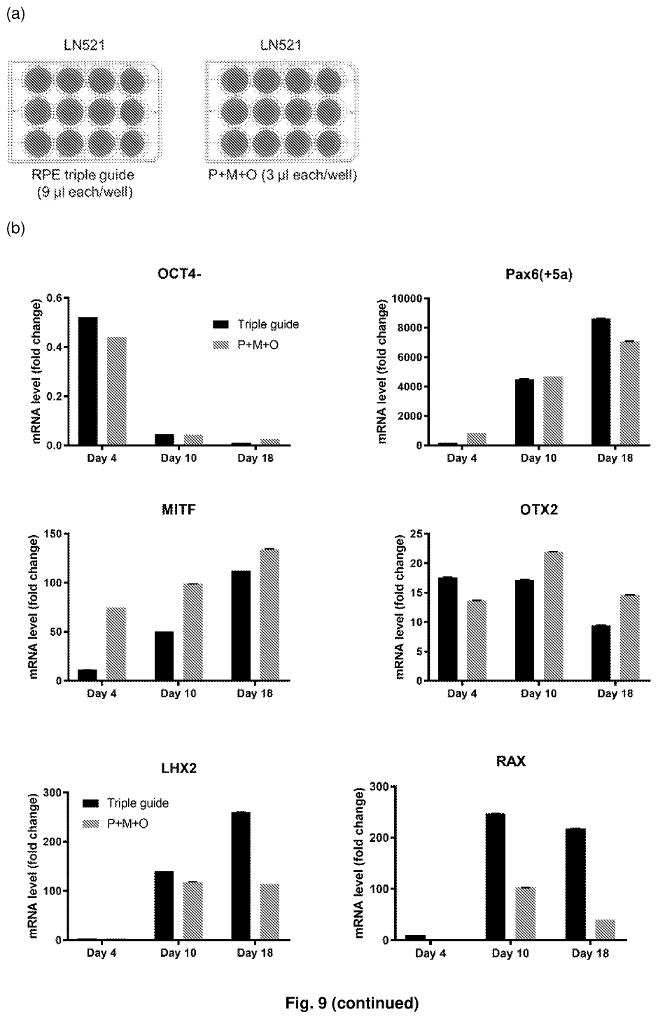

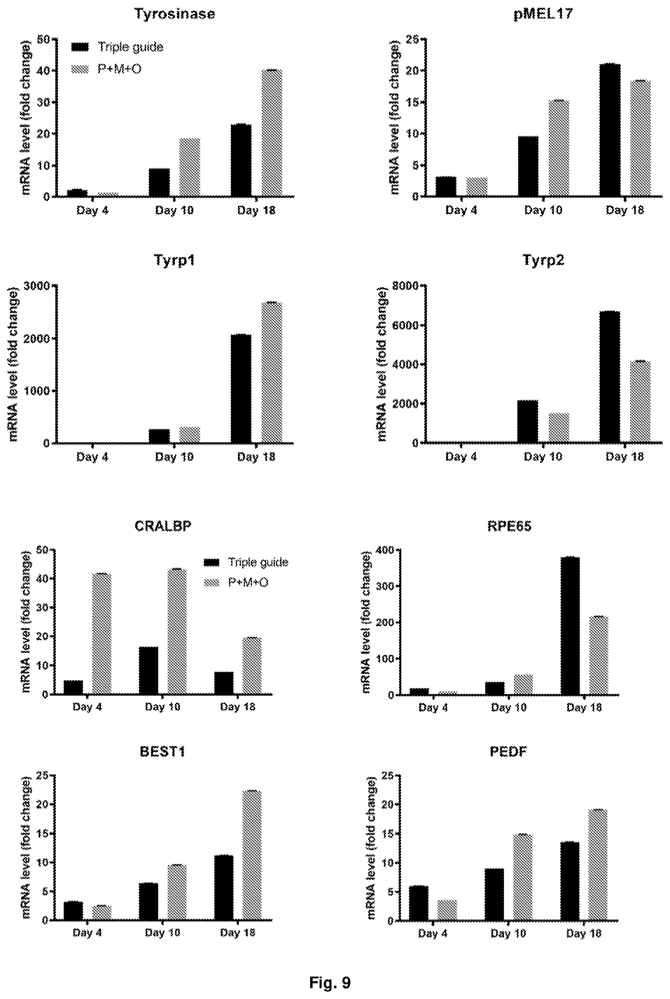

FIG. 9 Characterization of hiPSC RPE differentiation using triple guide lentivirus and the mix of individual, Pax6 (P), MITF (M) and OTX2 (0) lentiviruses. (a) Schematic representation of the experiment design. (b) Progression of gene expression profile of pluripotency marker (Oct4-), RPE differentiation early and mature markers. iPSC-CRISPR SAM cells were plated as single cells (as before) on day 0, qPCR data on days 4, 10 and 18. The error bars represent the standard error of the mean. Abbreviations: Oct-4 (Octamer-binding transcription factor 4), PAX6 (Paired box protein), OTX2 (Orthodenticle homeobox 2), MITF (Melanocyte Inducing Transcription Factor), pMEL17 (Melanocyte protein), TyrP1 (Tyrosinase Related Protein-1), CRALBP (Retinaldehyde-binding protein-1), RPE65 (Retinoid isomerohydrolase), BEST1 (Bestrophin-1), PEDF (Pigment epithelium-derived factor), TyrP2 (Tyrosinase Related Protein-2), LHX2 (LIM Homeobox 2), RAX (Retinal homeobox protein).

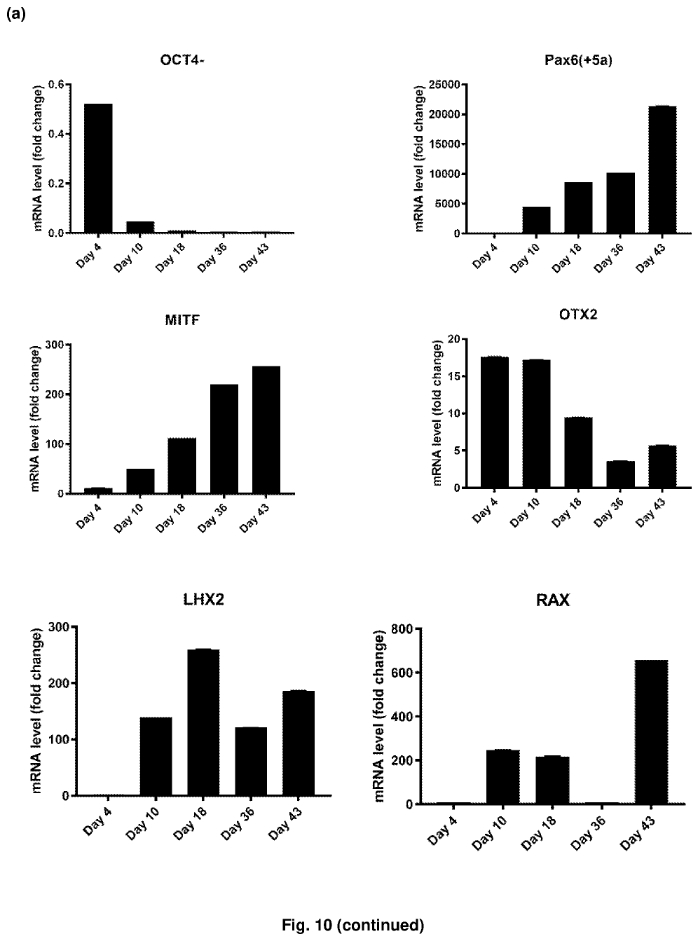

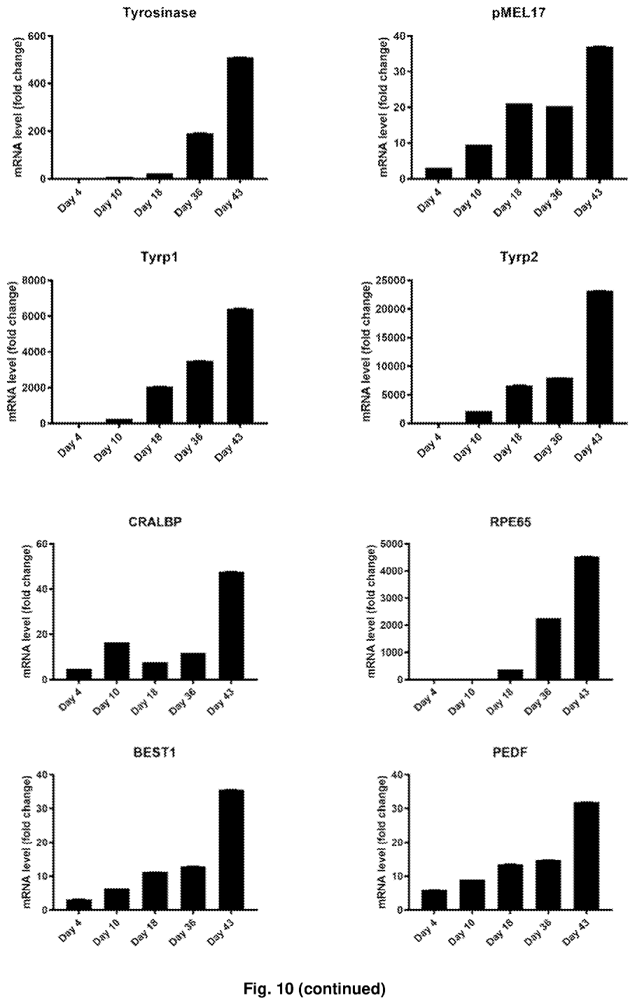

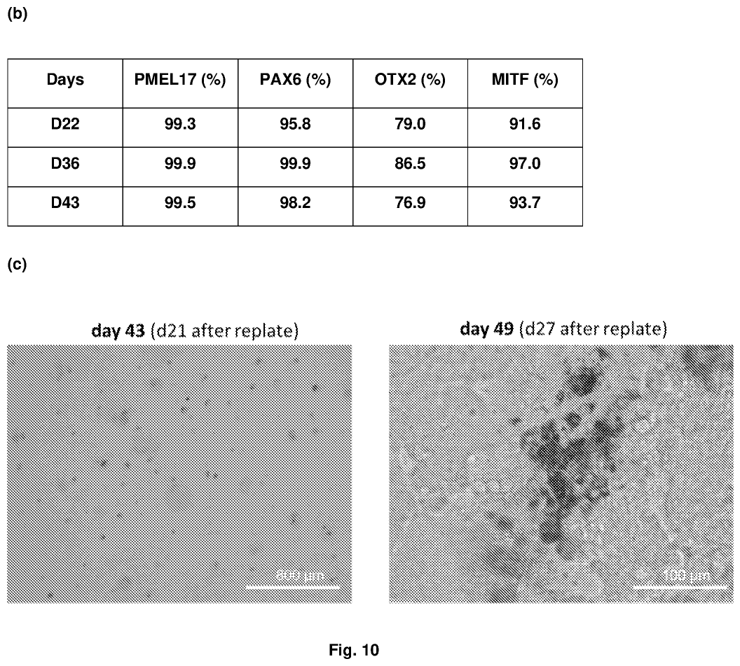

FIG. 10 Characterization of hiPSC RPE differentiation using triple guide lentivirus. (a) Time-course of gene expression progression of RPE markers. The error bars represent the standard error of the mean. (b) FACS analysis of the expression of RPE specific markers (% positive cells) during differentiation. (c) Pigmentation and cobble stone morphology of RPE cells are shown as phase contrast images. Abbreviations: Oct-4 (Octamer-binding transcription factor 4), PAX6 (Paired box protein), OTX2 (Orthodenticle homeobox 2), MITF (Melanocyte Inducing Transcription Factor), pMEL17 (Melanocyte protein), TyrP1 (Tyrosinase Related Protein-1), CRALBP (Retinaldehyde-binding protein-1), RPE65 (Retinoid isomerohydrolase), BEST1 (Bestrophin-1), PEDF (Pigment epithelium-derived factor), TyrP2 (Tyrosinase Related Protein-2), LHX2 (LIM Homeobox 2), RAX (Retinal homeobox protein), ZO-1 (Zonula occludens-1).

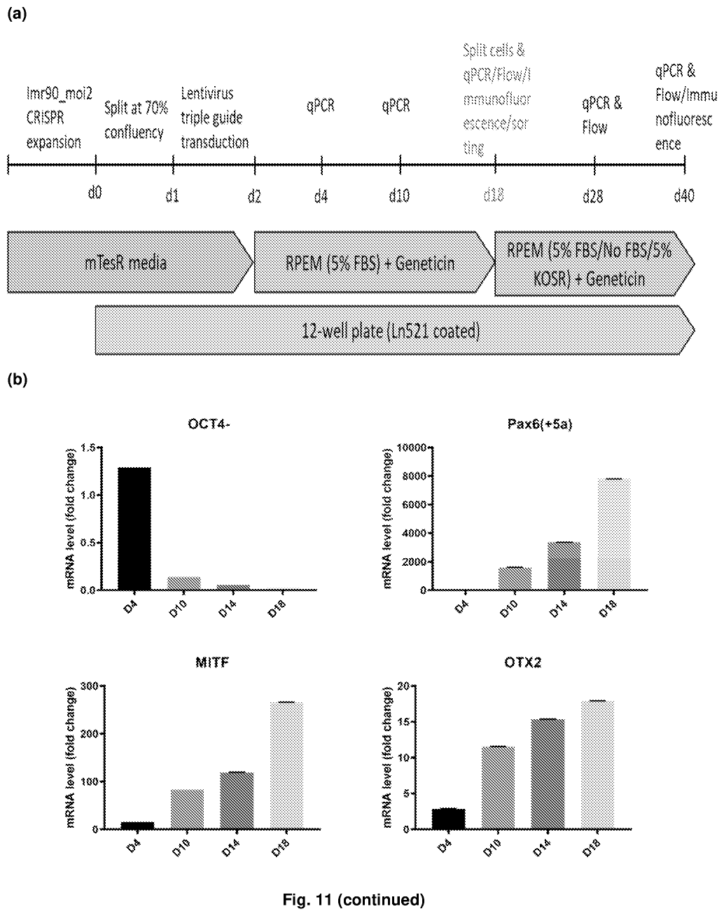

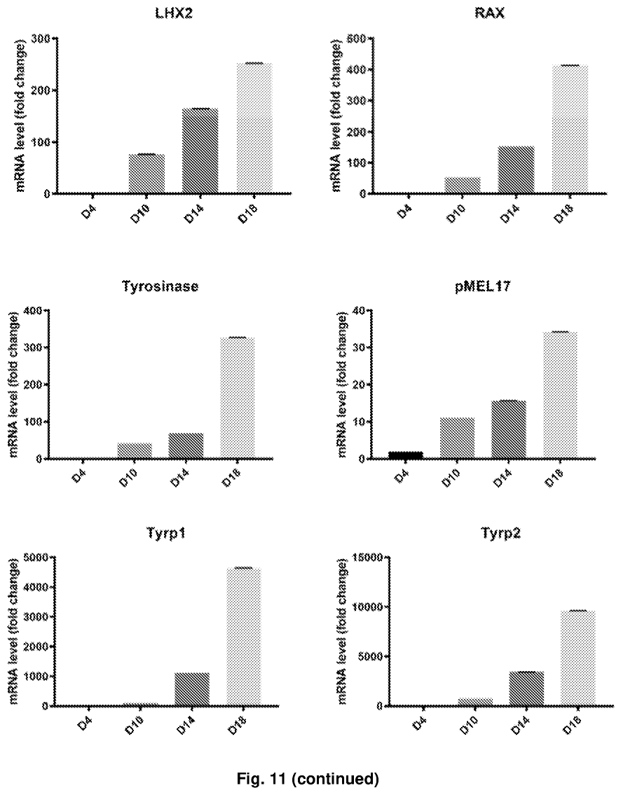

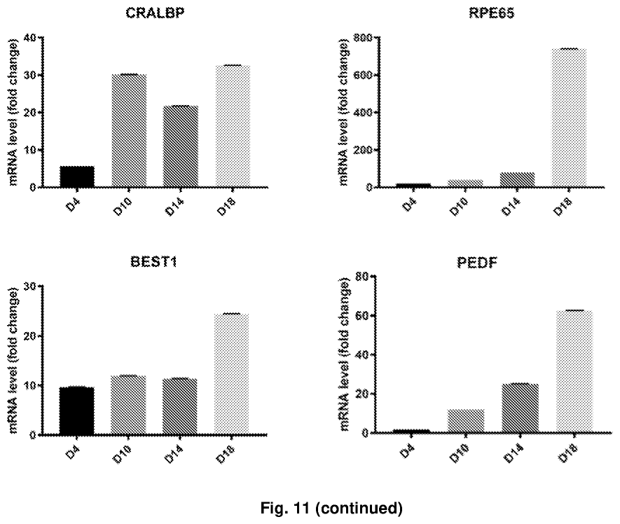

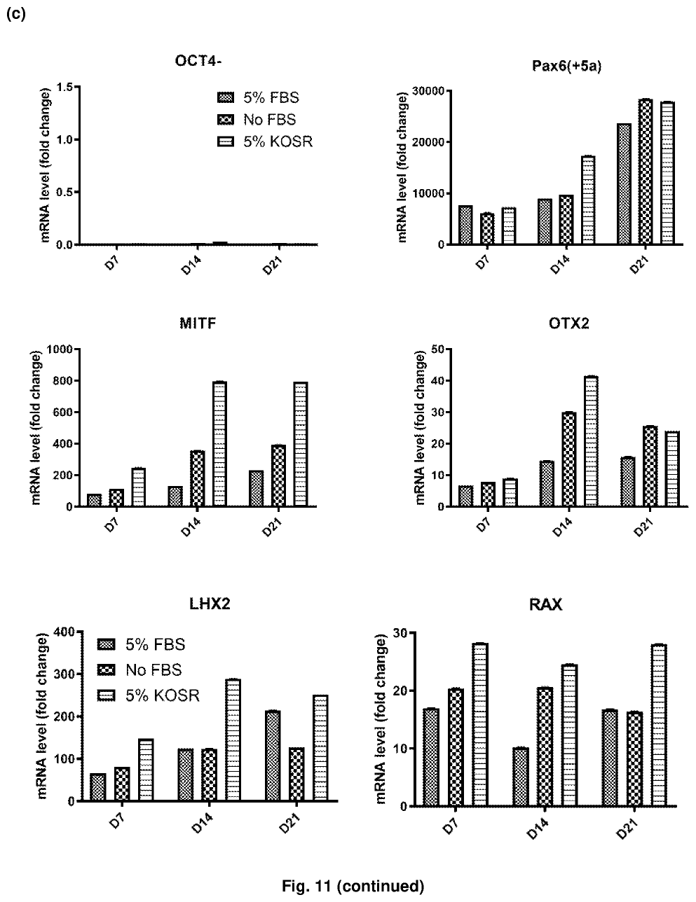

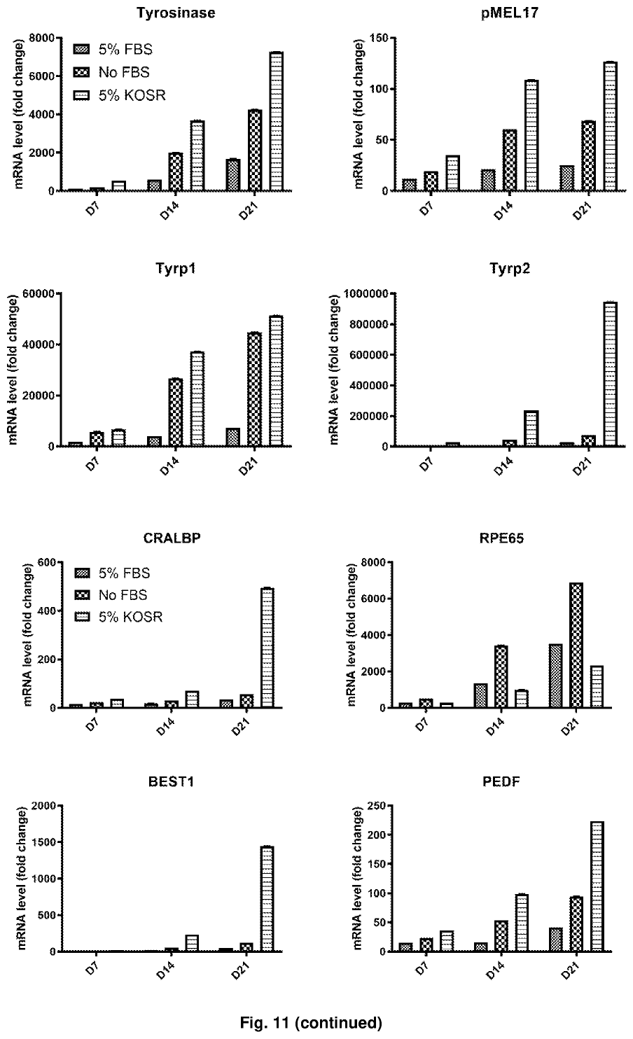

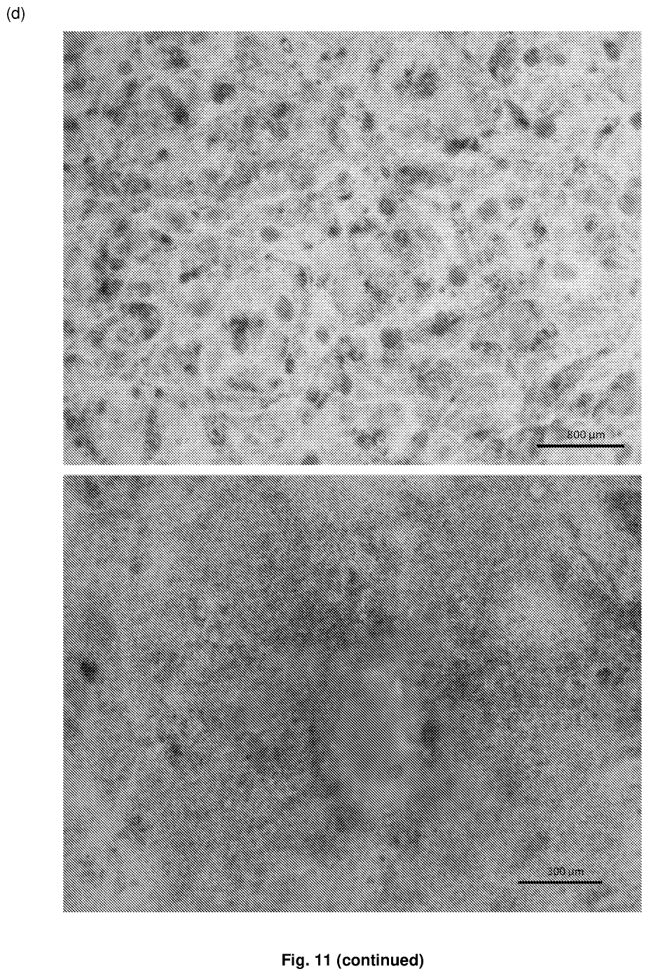

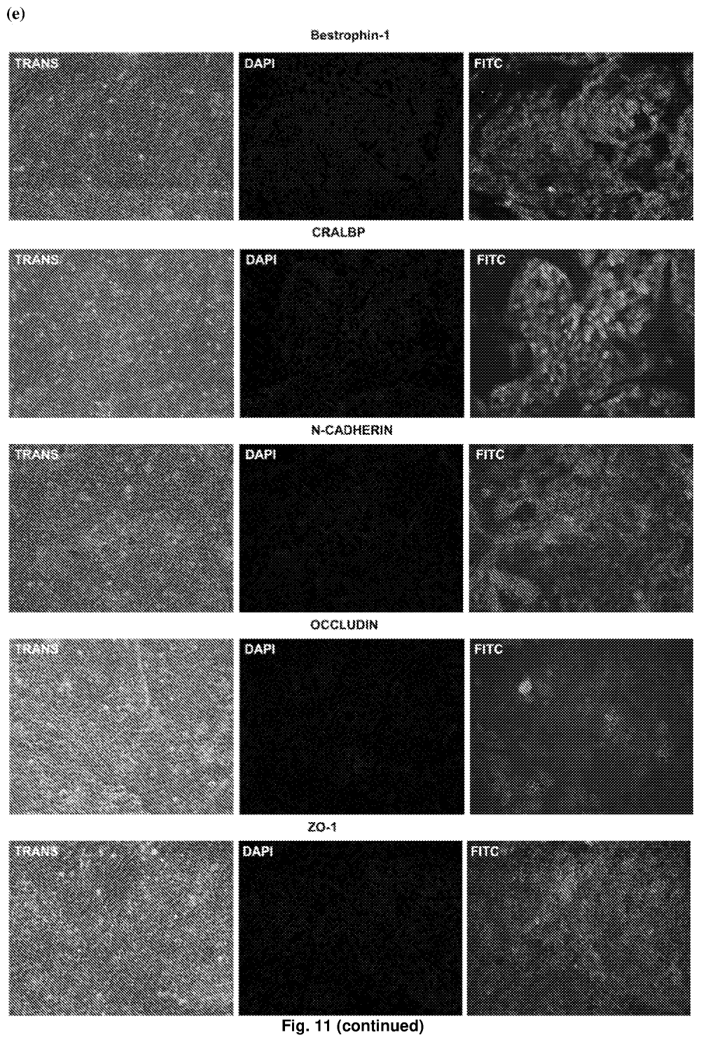

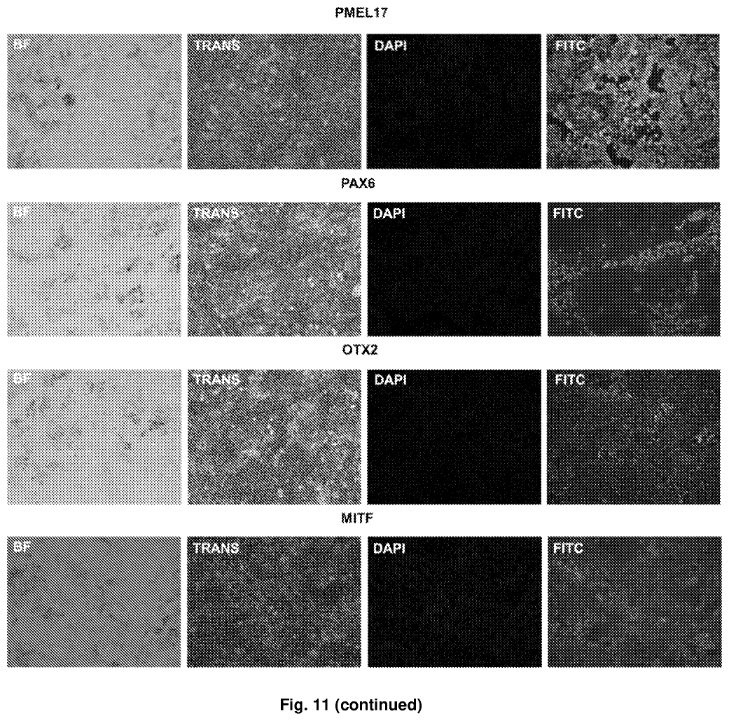

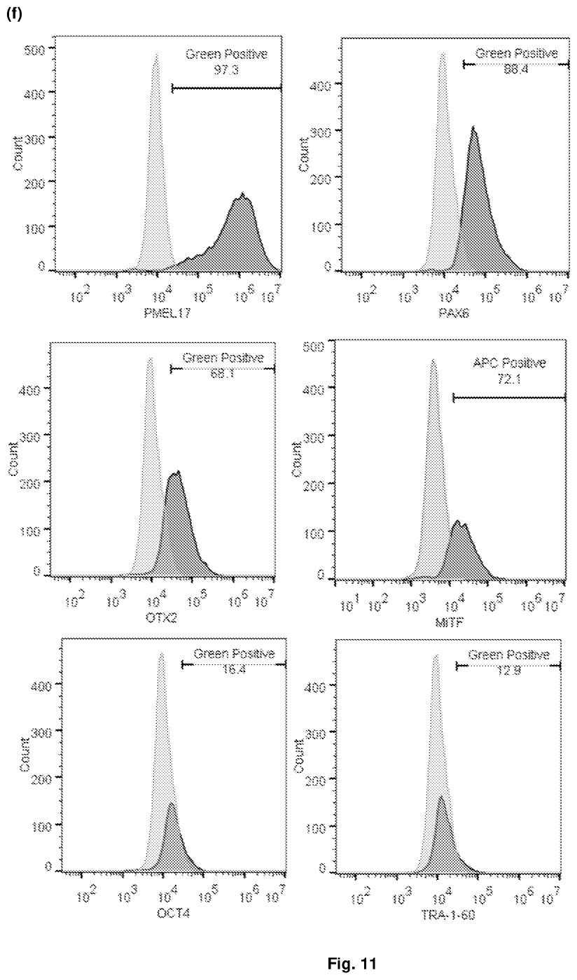

FIG. 11 Characterization of iPSC-dCas9 SAM cells differentiation into RPE cells using triple sgRNA lentivirus. (a) Schematic representation of the protocol and timeline for RPE differentiation. On day 18, the RPE progenitor cells were split and tested individually under three different conditions (5% FBS, No FBS and 5% KOSR). qPCR analysis comparing the time-course of eye field, early and mature RPE genes during RPE differentiation (b) days 4-18 after RPE triple virus transduction & (c) days 7-21 after the cells were split on day 18 under three different media conditions. The error bars represent the standard error of the mean. (d) Phase contrast images of iPSC-dCas9 SAM cells differentiated into RPE cells (pigmentation and cobble stone morphology) 21 days (p1) after replating on Dayl8 in 5% KOSR media. (e) Immunofluorescence images of mature RPE-specific tight junction markers (Bestrophin-1, CRALBP, N-Cadherin, Occludin & ZO-1), pigmentation marker (PMEL17) and CRISPR-activated RPE transcription factors (Pax6, Mitf & Otx2) from cells grown in 5% KOSR. Scale bar=100 μm. (f) Representation of flow cytometry histograms for RPE pigmentation marker (PMEL17), CRISPR-activated TFs (Pax6, Otx2, MITF) and pluripotency markers (Oct4- and TRA-1-60) at day 40 in 5% KOSR. Abbreviations: Oct-4 (Octamer-binding transcription factor 4), PAX6 (Paired box protein), OTX2 (Orthodenticle homeobox 2), MITF (Melanocyte Inducing Transcription Factor), pMEL17 (Melanocyte protein), TyrP1 (Tyrosinase Related Protein-1), CRALBP (Retinaldehyde-binding protein-1), RPE65 (Retinoid isomerohydrolase), BEST1 (Bestrophin-1), PEDF (Pigment epithelium-derived factor), TyrP2 (Tyrosinase Related Protein-2), LHX2 (LIM Homeobox 2), RAX (Retinal homeobox protein), ZO-1 (Zonula occludens-1).

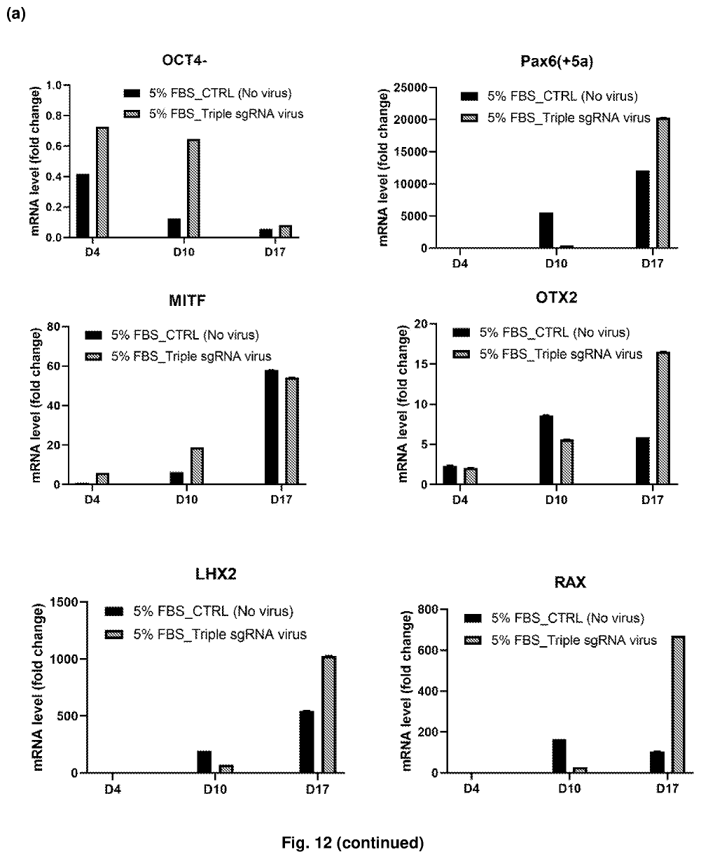

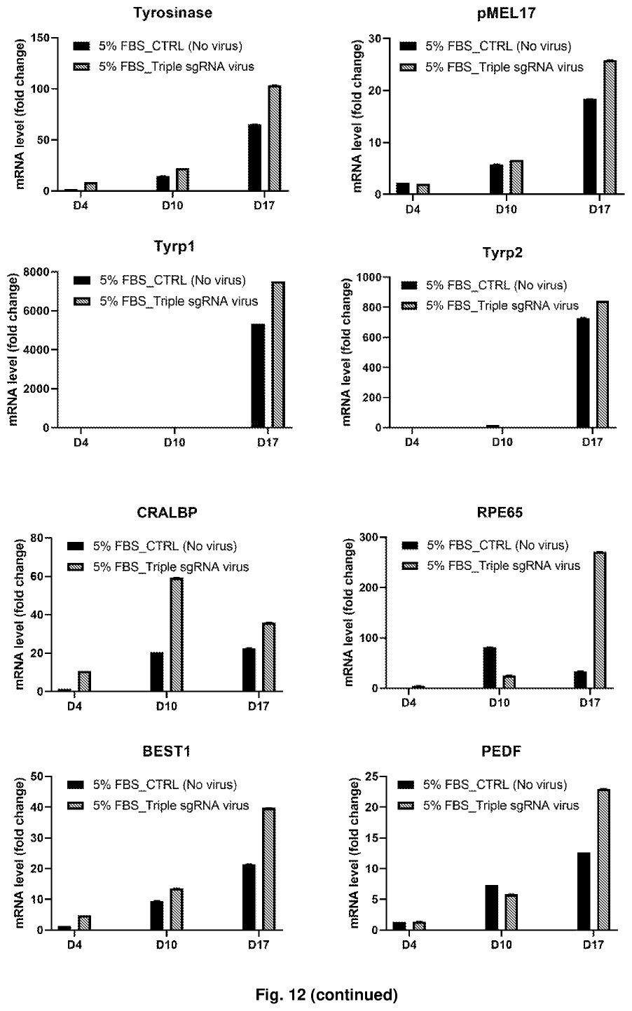

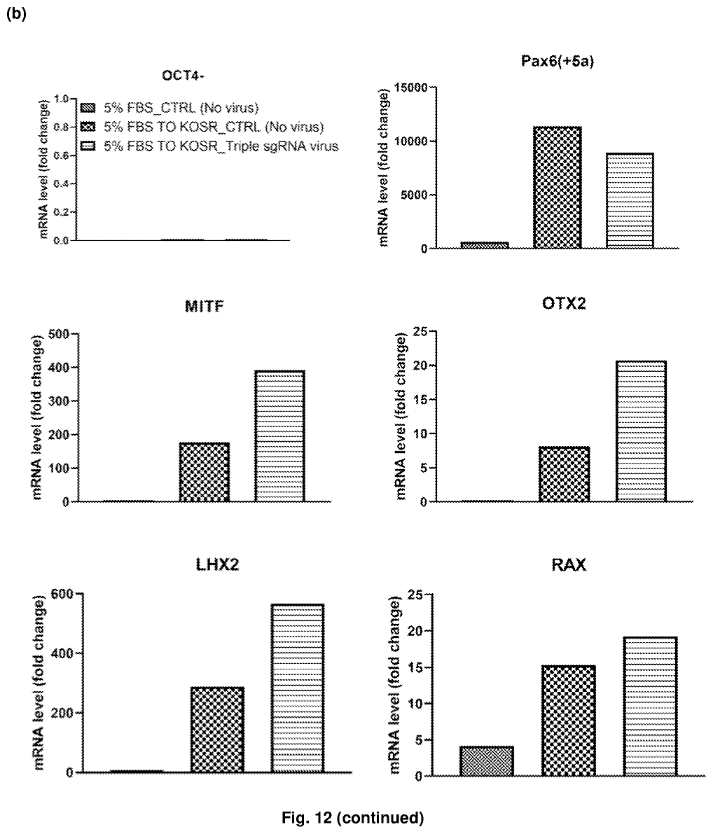

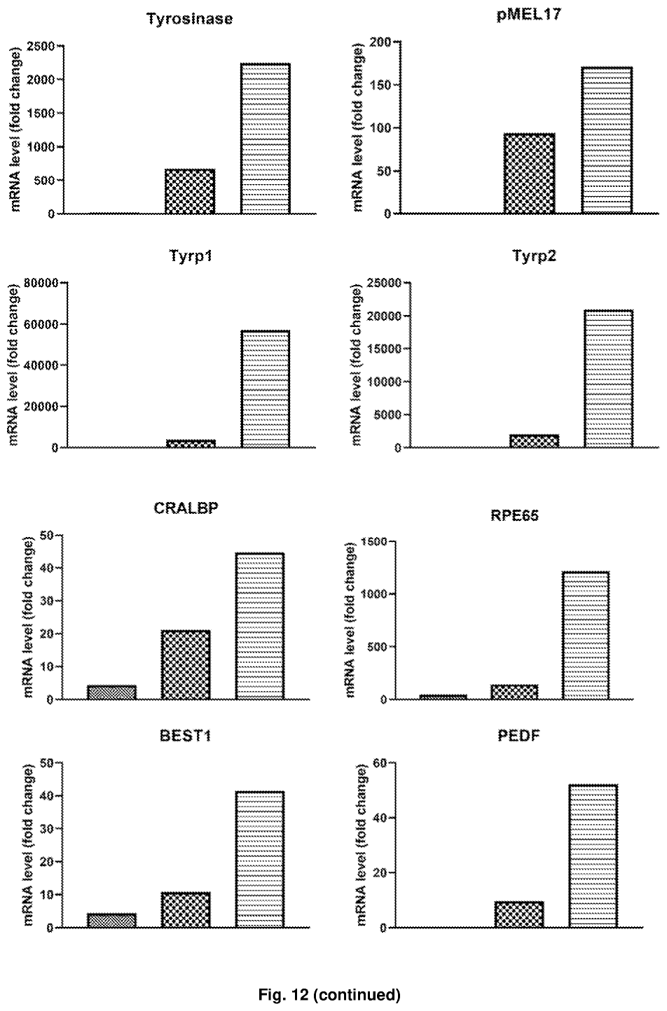

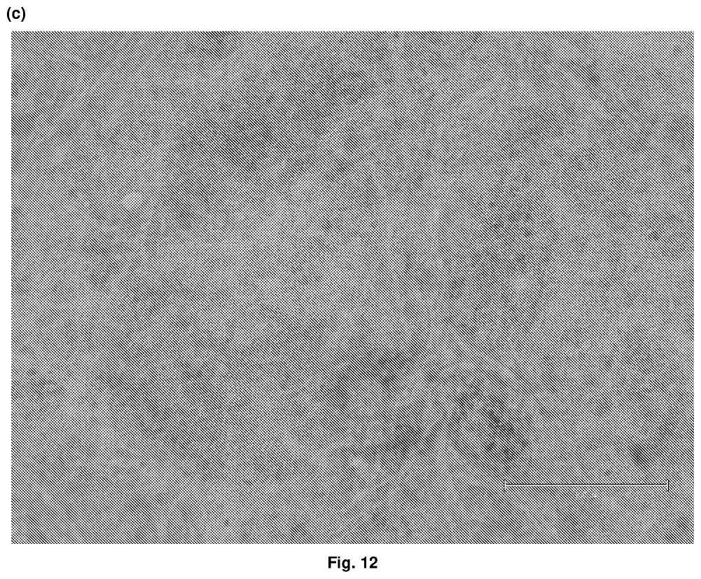

FIG. 12 Characterization of hESC-dCas9 SAM cells differentiation into RPE cells using triple sgRNA lentivirus. The protocol for iPSC-dCas9 SAM cells shown in FIG. 4 was validated using hESC-dCas9 SAM cells. qPCR analysis comparing the time-course of eye field, early and mature RPE genes during RPE differentiation (a) days 4-17 after RPE triple virus transduction & (b) day 11 after the cells were split on day 18 in 5% KOSR. For the control, cells (without virus transduction) were maintained in 5% FBS and after day 17 the cells were split and maintained in 5% FBS (5% FBS_ctrl) and 5% KOSR (5% FBS to 5% KOSR_ctrl). The error bars represent the standard error of the mean. (c) Phase contrast images of hESC-dCas9 SAM cells differentiated into RPE cells (pigmentation and cobble stone morphology) 21 days (p1) after replating on Dayl7. Abbreviations: Oct-4 (Octamer-binding transcription factor 4), PAX6 (Paired box protein), OTX2 (Orthodenticle homeobox 2), MITF (Melanocyte Inducing Transcription Factor), pMEL17 (Melanocyte protein), TyrP1 (Tyrosinase Related Protein-1), CRALBP (Retinaldehyde-binding protein-1), RPE65 (Retinoid isomerohydrolase), BEST1 (Bestrophin-1), PEDF (Pigment epithelium-derived factor), TyrP2 (Tyrosinase Related Protein-2), LHX2 (LIM Homeobox 2), RAX (Retinal homeobox protein), ZO-1 (Zonula occludens-1).

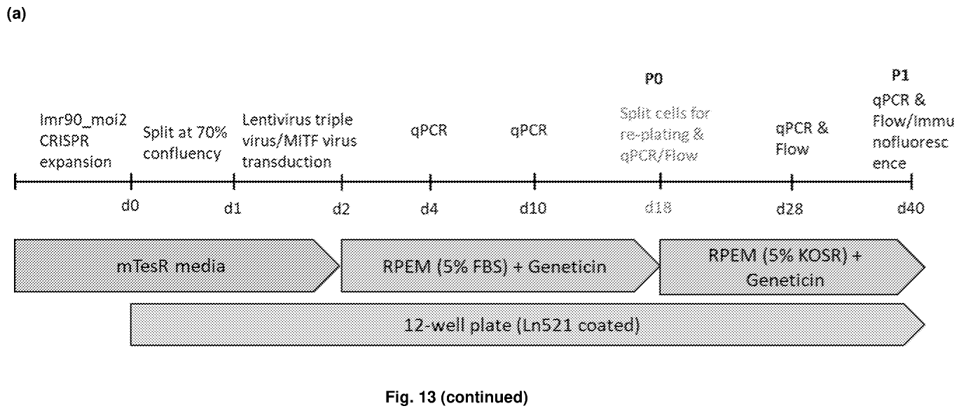

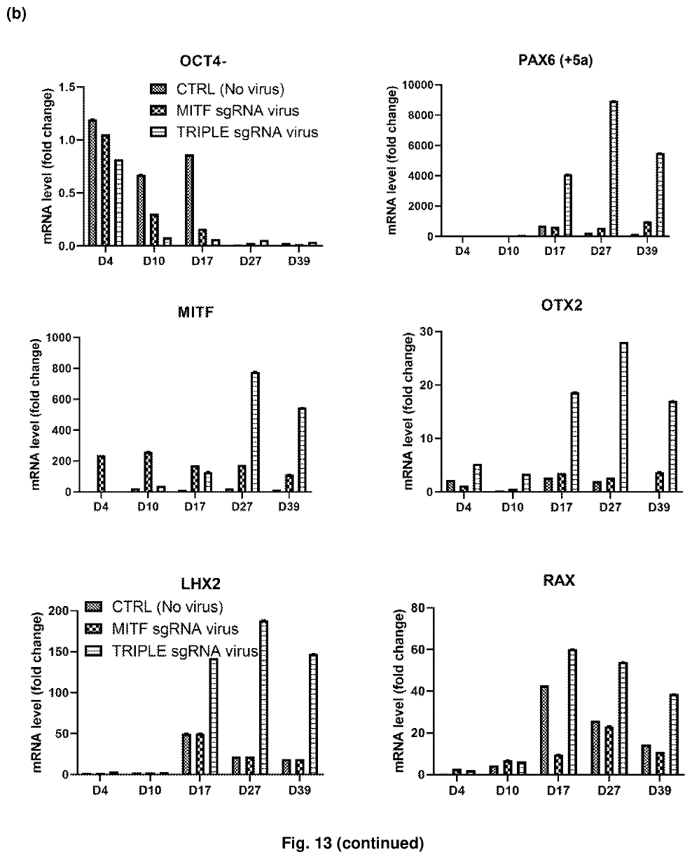

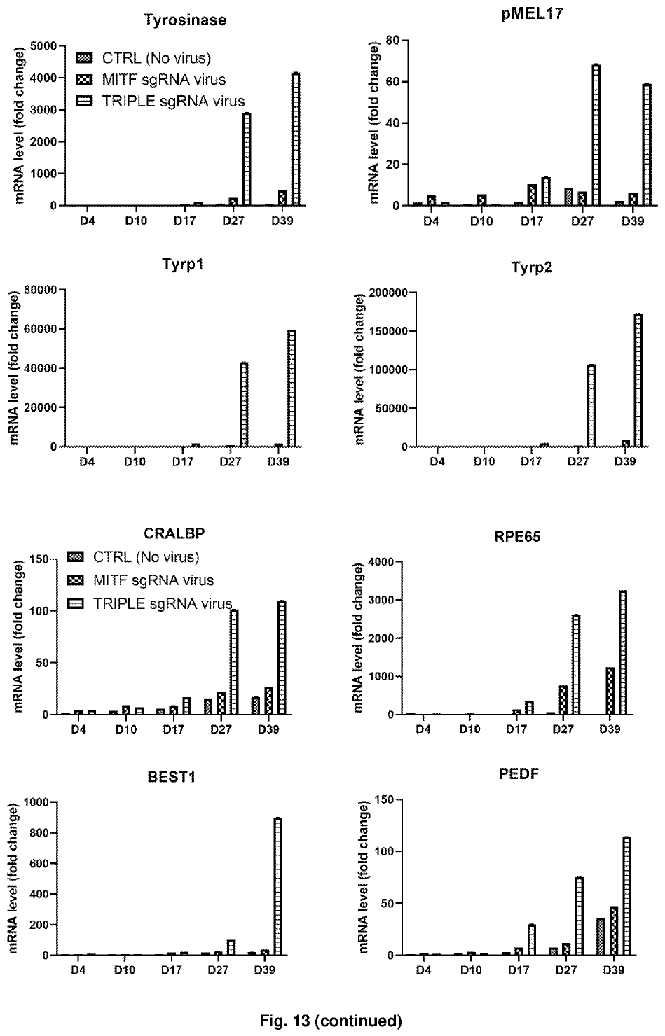

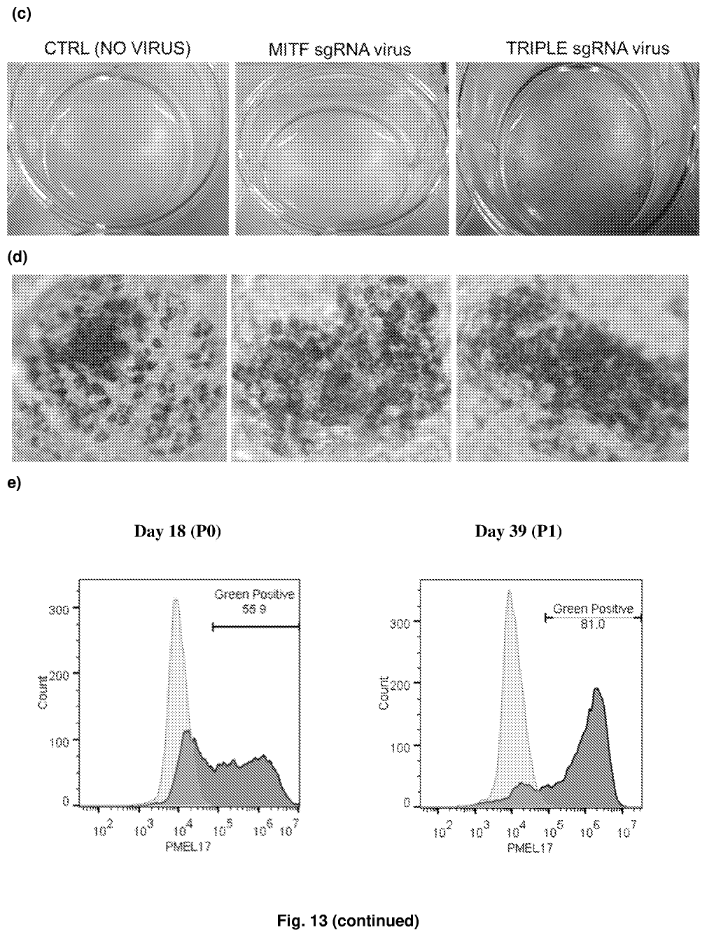

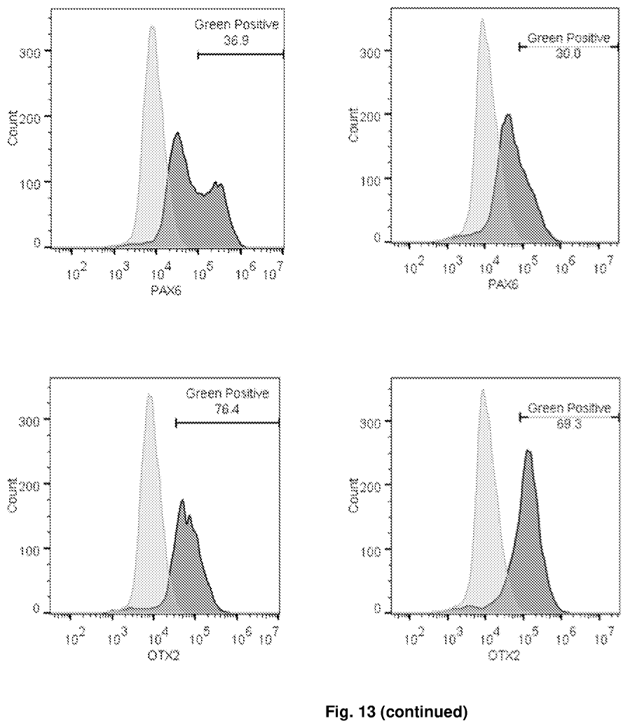

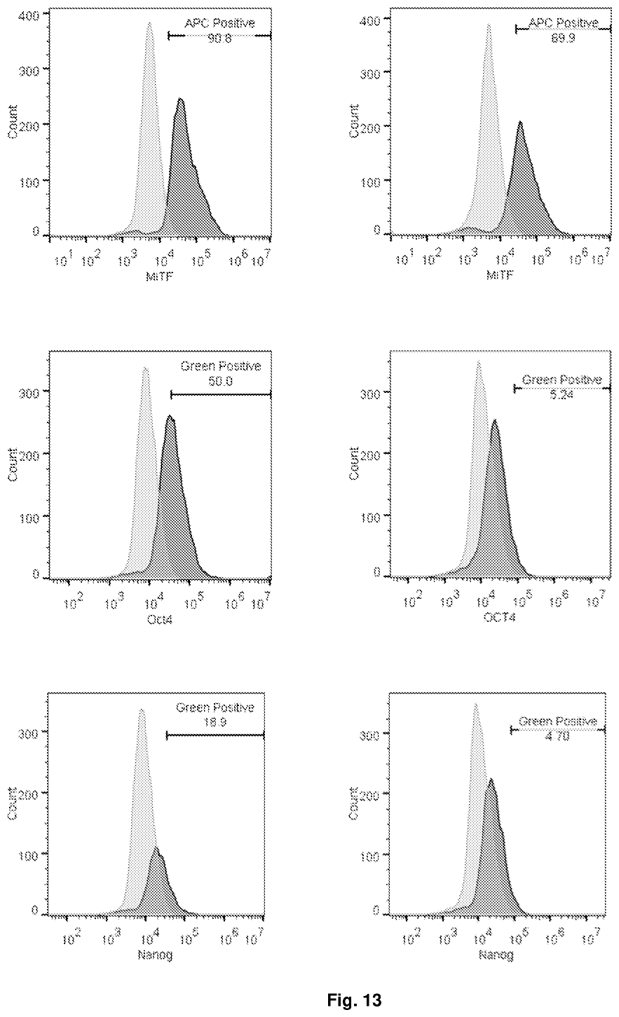

FIG. 13 Characterization of iPSC-dCas9 SAM cells differentiation into RPE using triple sgRNA lentivirus or MITF sgRNA lentivirus only. (a) Schematic representation of the protocol used for this study. (b) Time-course qPCR analysis showing progression of RPE differentiation from triple sgRNA lentivirus transduced cells, wherein the control (no virus) and MITF sgRNA lentivirus transduced cells have very low RPE markers expression. The error bars represent the standard error of the mean. Note triple sgRNA transduced cells shows higher RPE marker expression as compared to MITF sgRNA only and control cells. (c) Morphology of iPSC-dCas9 SAM cells after 39 days of differentiation. Note the absence of pigmented clusters in the control and MITF sgRNA lentivirus transduced wells. (d) Phase contrast microscopic images of iPSC-dCas9 SAM cells differentiated into RPE using triple sgRNA guide displaying the pigmented and cobblestone morphology on day 39. Scale bar: 100 μm. (e) Representative flow cytometry histograms of RPE and pluripotency markers expression on day 18 (P0) and day 39 (P1) cells transduced with triple sgRNA lentivirus. Abbreviations: Oct-4 (Octamer-binding transcription factor 4), PAX6 (Paired box protein), OTX2 (Orthodenticle homeobox 2), MITF (Melanocyte Inducing Transcription Factor), pMEL17 (Melanocyte protein), TyrP1 (Tyrosinase Related Protein-1), CRALBP (Retinaldehyde-binding protein-1), RPE65 (Retinoid isomerohydrolase), BEST1 (Bestrophin-1), PEDF (Pigment epithelium-derived factor), TyrP2 (Tyrosinase Related Protein-2), LHX2 (LIM Homeobox 2), RAX (Retinal homeobox protein).

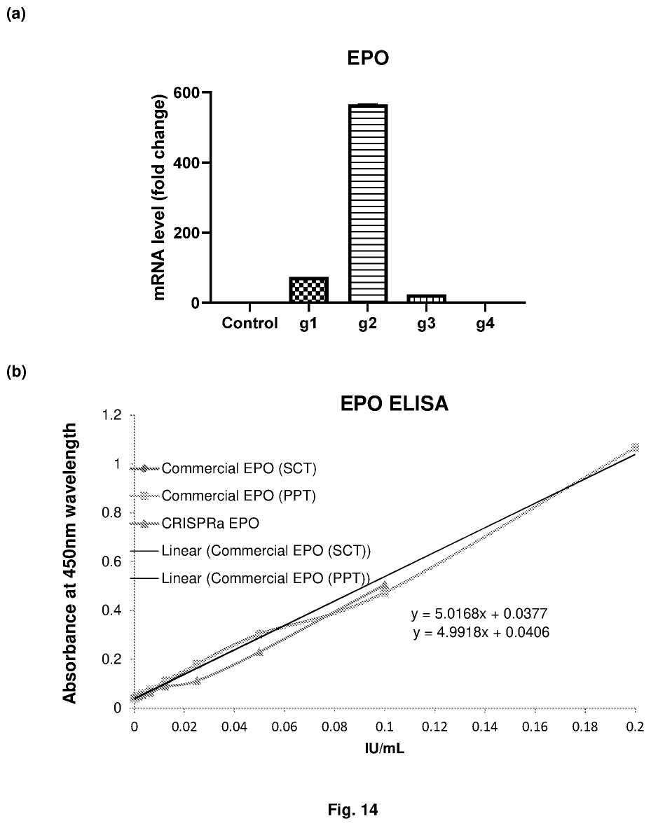

FIG. 14 Endogenous activation of erythropoietin (EPO) growth factor in human iPSC-dCas9 SAM cells. (a) qPCR data collected after 4 days of EPO sgRNAs lentivirus (unconcentrated) transduction. (b) EPO ELISA was carried out using two commercially available sources to quantify the CRISPRa EPO. For this, the spent mTesR medium of cells transduced with EPO_g2 were collected on days 3 and 4, the pooled medium was concentrated using Amicon Ultra-15 centrifugal filter (10 KDa cutoff membrane) since the EPO MW is 21 KDa. The retentate was used for quantification using ELISA. According to the standards, the EPO secreted in the medium is in the range of 47-51 IU/mL of EPO.

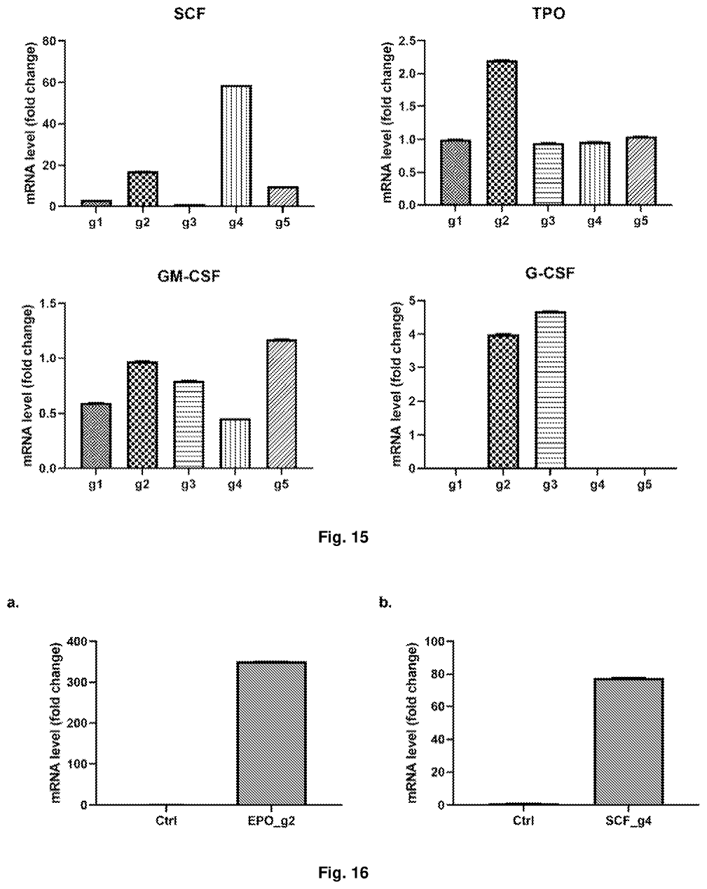

FIG. 15 Endogenous activation of growth factors in human iPSC-dCas9 SAM cells. qPCR data analysis of stem cell factor (SCF), thrombopoietin (TPO), granulocyte-macrophage colony-stimulating factor (GM-CSF) and granulocyte-colony stimulating factor (G-CSF).

FIG. 16 Endogenous activation of factors in HEK293_CRISPR dCas9 SAM cells. qPCR data analysis of (a) erythropoietin (EPO) and (b) stem cell factor (SCF) genes.

### EXAMPLES

Materials and Method

Establishment of Stable Cell Line:

To establish stably expressing CRISPR/dCas9-SAM expressing pluripotent stem cells, iPSC and hESC3 cells were plated on GelTrex-coated 96-well tissue culture plate at approximately 2.4×10⁴ cells in 110 μL of mTesR1 medium with 10 μM ROCK inhibitor. After 24 h, cells were transduced with the lentiviral vectors (FIG. 1a), dCas9-VP64 (Addgene: 61425) and MS2-p65-HSF1 (Addgene: 61426) together at multiplicity of infection (MOI=2) with 8 μg/mL polybrene (hexadimethrine bromide. 24 h after transduction, the culture medium was replaced with mTesR1 containing the selection antibiotics (Hygromycin B, 50 μg/mL and Blasticidin S, 4 μg/mL). The mTesR1 medium with antibiotics was replaced every day for 4-7 days, until there are no viable cells in the no-virus control. iPSC-CRISPR dCas9 SAM and hESC-CRISPR dCas9 SAM cells, were passaged an additional two days with mTesR1 medium without antibiotics and were subsequently passaged and banked accordingly.

Guide RNA Design and Plasmid Construction:

Guide RNAs were designed and assembled as described by Konermann et al³. For each gene, 5 sgRNA target sites spread across the proximal promoter between −200 bp to +1 bp window were selected. The sgRNA sequences are listed in Table 1. Briefly, the lentiviral vectors with different sgRNA sequences for each gene were generated by oligo cloning using the BsmBI site of lenti sgRNA(MS2)_zeo backbone (Addgene: 61427). Primers were supplied by Integrated DNA Technologies, IDT (Singapore) and sequences were verified through Axil Scientific Pte Ltd. (1st BASE, Singapore). The primer sequences are listed in Table 2.

TABLE 1
List of sgRNA sequences designed for this study.

| | | | | | Specificity | Efficiency | |
|---|---|---|---|---|---|---|---|
| Name | Position | Strand | Sequence | PAM | Score | Score | Distance |
| PAX6_4 | 31811163 | 1 | AATGTGTGTGTGCCGGCGCC | CGG | 85.71532 | 45.53779 | 130 |
| PAX6_3 | 31811122 | 1 | GCCAGCACACCTATGCTGAT | TGG | 80.41661 | 57.03529 | 89 |
| PAX6_5 | 31811191 | −1 | GCTTCGCTAATGGGCCAGTG | AGG | 74.83166 | 67.60208 | 172 |
| PAX6_1 | 31811054 | 1 | ACAATAAAATGGGCTGTCAG | CGG | 59.43153 | 66.63383 | 21 |
| PAX6_2 | 31811082 | 1 | GAGTGAGAGATAAAGAGTGT | GGG | 51.10384 | 64.62191 | 49 |
| MITF_1 | 69739390 | 1 | CGGGCCGAACTACAGATCCC | AGG | 87.69096 | 59.49774 | 42 |
| MITF_2 | 69739276 | 1 | CCAAACAGGAGTTGCACTAG | CGG | 83.95155 | 58.15318 | 94 |
| MITF_4 | 69739338 | 1 | AGCTGTAGTTTTCGTGGGAG | CGG | 77.456 | 53.51254 | 156 |
| MITF_3 | 69739291 | −1 | GCGGGGGAGAGGCAACGTGG | TGG | 69.70525 | 62.74833 | 127 |
| MITF_5 | 69739214 | 1 | CTGTACCCTTGAAGCAAGTG | GGG | 66.46342 | 65.68941 | 218 |
| OTX2_2 | 56810650 | −1 | GAACATTCTGGTAATGTCGG | AGG | 88.91548 | 64.17876 | 65 |
| OTX2_1 | 56810495 | −1 | GCGTCAAAAAGTTGCCAGAG | AGG | 76.19507 | 66.72045 | 15 |
| OTX2_4 | 56810615 | 1 | AACAGGCCGCTGCTGCACGG | GGG | 71.60974 | 62.94672 | 121 |
| OTX2_5 | 56810559 | 1 | GATTGACACATCTAAGCCAG | AGG | 69.85061 | 64.23791 | 170 |
| OTX2_3 | 56810590 | 1 | TAAAAACACACAACAGGGGG | AGG | 64.48448 | 55.39254 | 96 |

TABLE 2
List of Primer sequences used for sgRNA
cloning protocol.

| Seq | | |
|---|---|---|
| ID No. | Name | Sequence |
| 16 | Pax6_1_Fwd | CACCGACAATAAAATGGGCTGTCAG |
| 17 | Pax6_1_Rev | AAACCTGACAGCCCATTTTATTGTC |
| 18 | Pax6_2_Fwd | CACCGGAGTGAGAGATAAAGAGTGT |
| 19 | Pax6_2_Rev | AAACACACTCTTTATCTCTCACTCC |
| 20 | Pax6_3_Fwd | CACCGGCCAGCACACCTATGCTGAT |
| 21 | Pax6_3_Rev | AAACATCAGCATAGGTGTGCTGGCC |
| 22 | Pax6_4_Fwd | CACCGAATGTGTGTGTGCCGGCGCC |
| 23 | Pax6_4_Rev | AAACGGCGCCGGCACACACACATTC |
| 24 | Pax6_5_Fwd | CACCGGCTTCGCTAATGGGCCAGTG |
| 25 | Pax6_5_Rev | AAACCACTGGCCCATTAGCGAAGCC |
| 26 | MITF_1_Fwd | CACCGCGGGCCGAACTACAGATCCC |
| 27 | MITF_1_Rev | AAACGGGATCTGTAGTTCGGCCCGC |
| 28 | MITF_2_Fwd | CACCGCCAAACAGGAGTTGCACTAG |
| 29 | MITF_2_Rev | AAACCTAGTGCAACTCCTGTTTGGC |
| 30 | MITF_3_Fwd | CACCGGCGGGGGAGAGGCAACGTGG |
| 31 | MITF_3_Rev | AAACCCACGTTGCCTCTCCCCCGCC |
| 32 | MITF_4_Fwd | CACCGAGCTGTAGTTTTCGTGGGAG |
| 33 | MITF_4_Rev | AAACCTCCCACGAAAACTACAGCTC |
| 34 | MITF_5_Fwd | CACCGCTGTACCCTTGAAGCAAGTG |
| 35 | MITF_5_Rev | AAACCACTTGCTTCAAGGGTACAGC |
| 36 | OTX2_1_Fwd | CACCGGCGTCAAAAAGTTGCCAGAG |
| 37 | OTX2_1_Rev | AAACCTCTGGCAACTTTTTGACGCC |
| 38 | OTX2_2_Fwd | CACCGGAACATTCTGGTAATGTCGG |
| 39 | OTX2_2_Rev | AAACCCGACATTACCAGAATGTTCC |
| 40 | OTX2_3_Fwd | CACCGTAAAAACACACAACAGGGGG |
| 41 | OTX2_3_Rev | AAACCCCCCTGTTGTGTGTTTTTAC |
| 42 | OTX2_4_Fwd | CACCGAACAGGCCGCTGCTGCACGG |
| 43 | OTX2_4_Rev | AAACCCGTGCAGCAGCGGCCTGTTC |
| 44 | OTX2_5_Fwd | CACCGGATTGACACATCTAAGCCAG |
| 45 | OTX2_5_Rev | AAACCTGGCTTAGATGTGTCAATCC |

Lentivirus Production:

HEK293T cells were cultured in D10 medium at 37° C. with 5% CO₂ and was maintained according to the manufacturer's recommendation. D10 recipe: Dulbecco's modified Eagle's medium (DMEM), Fetal bovine serum, heat-inactivated (10%), Penicillin G (100 units/mL) and Streptomycin (100 μg/mL). Cells were seeded into T175 flasks 20-24 h at a density of 1.8×10⁷ cells per flask in a total volume of 37 mL of D10 medium. Transfection was carried out using Lipofectamine 3000 reagent according to manufacturer's recommendation. Briefly, Lipofectamine 3000 reagent, lentivirus packaging plasmids (pMD2.G (8 μg)+pMDLg/pRRE (8 μg)+pRSV-Rev (8 μg)) and lenti expression vector (15 μg) with P3000 reagent were diluted in Opti-MEMTM I medium and were incubated for 10 min in room temperature. The solution mix with 50% of the A10 media was added directly to the cells and after 4h the medium was replaced with fresh pre-warmed D10 medium. Virus supernatant was harvested twice at 48 h and 72 h post transfection, and then filtered with a 0.45 μm PVDF filter (Millipore).

sgRNA Screening for Endogenous Gene Activation:

iPSC-CRISPR dCas9 SAM were plated at approximately 1×10⁵ cells/well in GelTrex-coated 12-well plate containing 1 ml of mTesR1 medium with 10 μM ROCK inhibitor. After 24 h, media was replaced with 0.5 mL of fresh mTesR1 media and 0.5 mL of top four sgRNA lentivirus supernatants of each gene target were added independently in different wells with 8 μg/mL polybrene. Fresh mTesR1 media with selection antibiotic, Zeocin (10 μg/ml) was replaced 24 h after transduction with daily media replenishments. Four days after transduction, cells total RNA samples were extracted for quantitative PCR analysis using Direct-zol RNA Miniprep kit (Zymo Research, CA).

sgRNA Lentivirus Concentration:

Top three performing guides were chosen for each gene and the concentration of the viral supernatants was carried out using Lenti-X-concentrator (Clontech) as per manufacturer's protocol. Briefly, for each guide viral supernatants from three T175 flasks were pooled and then filtered with a 0.45 μm PVDF filter (Millipore). To one volume of Lenti-X-concentrator, 3 volumes of clarified lentivirus supernatant was added and incubated at 4° C. for 30-45 min. The sample were centrifuged at 1500×g for 45 min at 4° C. The pellet was dissolved in 2.5 ml of sterile PBS and was stored at −80° C. in single-use aliquots.

Endogenous Gene Activation Using Concentrated sgRNA Lentivirus:

iPSC-CRISPR dCas9 SAM were plated at approximately 2×10⁴ cells/well in GelTrex-coated 12-well plate containing 1 ml of mTesR1 medium with 10 μM ROCK inhibitor. After 24 h, media was replaced with 1 mL of fresh mTesR1 and 3 μl of crude concentrated sgRNA lentivirus of each gene target were added independently in different wells with 8 μg/mL polybrene. For simultaneous activation of three genes, the ratio of sgRNAs targeting each gene was 1:1:1. Fresh mTesR1 media with selection antibiotic, Zeocin (10 μg/ml) was replaced 24 h after transduction with daily media replenishments. Four days after transduction, cells total RNA samples were extracted for quantitative PCR analysis using Direct-zol RNA Miniprep kit (Zymo Research, CA).

RPE Induction Using Gene Activation:

iPSC-CRISPR dCas9 SAM were plated at approximately 1×10⁵ cells/well in GelTrex-coated 12-well plate containing 1 ml of mTesR1 medium with 10 μM ROCK inhibitor. After 24 h, media was replaced with 1 mL of fresh mTesR1 and 3 μl of crude concentrated top performing sgRNA lentivirus of each gene target were added together with 8 μg/mL polybrene. 24 h after transduction, the medium was changed to RPE maintenance medium (RPEM) for 4-6 weeks with medium change twice a week. Samples were collected at different time points for quantitative PCR and flow cytometry analysis.

Quantitative Real-Time Polymerase Chain Reaction:

cDNA was synthesized from 1 μg of RNA using the Maxima First Strand cDNA Synthesis Kit (ThermoFisher). Quantitative real-time polymerase chain reaction (qPCR) was carried out using QuantStudio 3 Real-Time PCR System (ThermoFisher). The samples were run in biological triplicates and expression levels normalized using the geometric mean of the “housekeeping” gene: glyceraldehyde phosphate dehydrogenase (GAPDH). The primer sequences are listed in Table 3.

TABLE 3
Primers for gene expression analysis used in this study.

| | Forward | Reverse | Ref. |
|---|---|---|---|
| Oct3/4 | CAGTGCCCGAAACCCACAC | GGAGACCCAGCAGCCTCAAA | 4 |
| Lhx2 | CGTCCGTCTTAACTTCTGTGC | AGGTTGGTAAGAGTCGTTTGT | |
| Rax | TCCCAGGAGGCTTGGAGACCC | CTCCCCAAGTCCTGAGCGTGC | |
| Otx2 | ACTTCCGAGAGCCATAGAAGG | TAAGCAGATTGGTTTGTCCAT | |
| Pax6(+5a) | CTCGGTGGTGTCTTTGTCAAC | ACTTTTGCATCTGCATGGGTC | 5 |
| Mitf | CCCAGTTCATGCAACAGAGAG | GCAGAGGGAAGGGTGGTG | |
| Tyrosinase | GTGTAGCCTTCTTCCAACTCAG | GTTCCTCATTACCAAATAGCATCC | |
| Tyrp1 | GATTCCACTCTAATAAGCCCAAA | TTCCAAGCACTGAGCGACAT | |
| Tyrp2 | CTCAGACCAACTTGGCTACAGCTA | CAGCACAAAAAGACCAACCAAA | |
| pmel17 | TGATGGCTGTGGTCCTTGC | CAGTGACTGCTGCTATGTGG | |
| PEDF | TATCACCTTAACCAGCCTTTCATC | GGGTCCAGAATCTTGCCAATG | |
| Best1 | TAGAACCATCAGCGCCGTC | TGAGTGTAGTGTGTATGTTGG | |
| Cralbp | CACGCTGCCCAAGTATGATG | CCAGGACAGTTGAGGAGAGG | |
| RPE65 | CCTGATTCATACCCATCAGAACCC | CACCACACTCAGAACTACACCATC | |
| GAPDH | AGCAAGAGCACAAGAGGAAGAG | GAGCACAGGGTACTTTATTGATGG | 6 |

Flow Cytometry:

The samples were fixed in 4% paraformaldehyde and permeabilized with 0.1% Triton X-100. The samples (1×10⁵ cells) were incubated with primary (pmell7 (DAKO, DKO.M063429), Pax6 (DSHB), Mitf (Abcam, ab122982) and Otx2 (Merck, SAB5300043)) or isotype control antibodies at 1:100 concentration for 30 minutes at room temperature. Primary and isotype control were labeled with fluorophore conjugated secondary antibodies and control cells were incubated with only the secondary antibody for 30 minutes at room temperature. The labeled samples were run on NovoCyte 2000 flow cytometer (ACEA, Biosciences, Inc.). Data analysis was performed using FlowJo V10 software. The positive percentage was based on a background level set at 1% positive expression in samples labeled with isotype control antibodies.

Triple sgRNA Design

In order to improve the RPE induction efficiency in iPSC-CRISPR dCas9 SAM cells, a single vector encoding all three Pax6, Mitf and Otx2 sgRNA sequences was designed. Each of the sgRNA sequence with the MS2 scaffold expression is driven using the U6 promoter upstream of the sgRNA sequence (FIG. 8). The lentiviral vector construction service was provided by Vector Biolabs, USA. The design of the lentiviral vector backbone was carried out using their custom web-based lentiviral vector design tool. The custom built triple sgRNA encoded lentiviral vector was used to produce concentrated lentiviral particles as described earlier.

The lentiviral vector backbone encodes geneticin as an antibiotic selection marker. A minimum inhibitory concentration (MIC) assay for geneticin using iPSC-CRISPR dCas9 SAM cells was carried out and the MIC of geneticin was found to be 100 μg/mL.

Endogenous Gene Activation Study Using Triple sgRNA Lentivirus

iPSC-CRISPR dCas9 SAM were plated at approximately 1×10⁵ cells/well in GelTrex-coated 12-well plate containing 1 mL of mTesR1 medium with 10 μM ROCK inhibitor. After 24 h, media was replaced with 1 mL of fresh mTesR1 and different concentrations of crude concentrated triple sgRNA lentivirus was added together with 8 μg/mL polybrene. Fresh mTesR1 media with selection antibiotic, Geneticin (100 μg/ml) was replaced 24 h after transduction with daily media replenishments. Four days after transduction, cells total RNA samples were extracted for quantitative PCR analysis using Direct-zol RNA Miniprep kit (Zymo Research, CA).

RPE Induction Using Triple sgRNA Lentivirus

iPSC-CRISPR dCas9 SAM/hESC-CRISPR dCas9 SAM were plated at approximately 1×10⁵ cells/well in Laminin 521-coated 12-well plate containing 1 mL of mTesR1 medium with 10 μM ROCK inhibitor. After 24 h, media was replaced with 1 mL of fresh mTesR1 and 9 μL of crude concentrated triple sgRNA lentivirus was added together with 8 μg/mL polybrene. 24 h after transduction, the medium was changed to RPE maintenance medium (RPEM) for 2 weeks with medium change twice a week. On day 17/18 after transduction, the cells (RPE progenitors) were passaged and re-plated at 4×10⁵ cells/well in Laminin 521-coated 12-well plate containing RPE maintenance medium (RPEM) for 3 weeks with medium change twice a week. Samples were collected at different time points for quantitative PCR and flow cytometry analysis.

Immunofluorescence Assay

Cells were fixed with 4% paraformaldehyde in PBS and were blocked and permeabilized for 30 min in 10% goat serum and 2 ml of 0.1% Triton X-100 in PBS, and then incubated overnight at 4° C. with the following primary antibodies at 1:100 concentration: mouse anti-Occludin (Thermo Fisher, 331500), mouse anti-ZO-1 (Thermo Fisher, 339100), mouse anti-BEST1 (Abcam, ab2182), mouse anti-N-Cadherin (Novus Biologicals, NBP1-48309), mouse anti-CRALBP (Abcam, ab15051), mouse anti-MITF (abcam, ab3201), anti-PAX6 (DSHB), anti-OTX2 (Merck, SAB5300043) and mouse anti-PMEL17 (DAKO, DKO.M063429). Cells were then incubated for 1 h at room temperature with the corresponding secondary antibody conjugated to Alexa-488 (Invitrogen) and counterstained with Hoechst 33342 (Invitrogen). Images were taken at room temperature with an epifluorescent microscope (Nikon Eclipse TE).

### Results

In order to overcome the above-mentioned problems, the present inventors came up with a hypothesis that endogenous activation of key transcription factors such as PAX6, MITF and OTX2 using CRISPR-dCas9/SAM will be enough to differentiate pluripotent stem cells into mature RPE tissue based on the knowledge search in the literature (FIG. 2). For this, sgRNA sequences (FIG. 3 and Table 2) were designed upstream of the target genes and top performing guide sequence were evaluated based on the maximal fold change achieved. After the initial screening, the present disclosure concentrated on the top performing candidates of sgRNA lentiviruses and demonstrated higher gene expression level (FIG. 4). Next, each of the top performing sgRNA lentiviruses of three target genes were added and the inventors were able to show multiplex activation of those genes in a single cell (FIG. 5). It was further showed that multiplexed endogenous activation of three genes and subsequent culture of cells in RPEM media resulted in pigmented, cobbled shaped foci of RPE cells at day 40 with all the RPE marker genes progressively upregulated over time. Importantly, the purity of the RPE population based on the PMEL17 expression is more than 96%. By this proposed method, a simple, robust and cost-effective protocol for RPE generation from pluripotent stem cells was achieved.

The RPE specific markers expression between day 21 hRPE (Lonza) and day 28 p1 of hiPSC-CRISPR/dCas9-SAM activated RPE cells were compared. The results demonstrated that iPSC-CRISPR activated RPE cells on day 28 gene expression patterns are similar to that of day 21 commercial human primary RPE cells (hRPE, Lonza) grown on laminin-coated 12-well plate (FIG. 7a). Specifically, the early eye gene markers Pax6, Mitf, Otx2, Lhx2 and Rax expression were similar to the hRPE cells. However, expression of pigmentation genes (Tyr and TyrP1) and mature markers (CRALBP, BEST1 and PEDF) expression were markedly higher in hRPE cells compared to the CRISPR activated RPE cells (FIG. 7a).

In order to improve the efficiency of RPE differentiation, the differentiation and expansion of iPSC-CRISPR activated RPE cells were tested on Laminin-521 (Ln521) coated 12-well plates. The efficiency of RPE marker expression of iPSC-CRISPR activated RPE cells were compared in both geltrax and Ln521 coated plates. The results show that Ln521 efficiently supports RPE differentiation (FIG. 7b) with robust expression of early eye-field genes (Pax6, Mitf, Otx2, Lhx2 and Rax), pigmentation genes (pmell7 & Tyrp2) and mature RPE markers (PEDF and BEST1). Based on this data, Ln521 coating was used for further studies.

Based on these studies (FIGS. 6 and 7), it is quite evident that activating the transcription factors (Pax6, Mitf and Otx2) in hiPSC cells can commit the cells towards RPE lineage. However, the uniformity and efficiency of RPE generation is hindered because of the addition of three individual sgRNAs in a cocktail possibly because different cells might receive the combination of different sgRNA dosage and fraction.

In order to overcome this, all three sgRNAs were incorporated in a single lentiviral vector as shown in FIG. 8a. This would allow each cell to activate all three transcription factors in unison. To identify the optimal concentration of triple sgRNA lentiviral infection, the iPSC-CRISPR dCas9 SAM cells were transduced with the triple sgRNA lentivirus at different concentrations. It was found that transduction using 9 μL of concentrated triple sgRNA virus yielded optimal expression of all three transcription factors (FIG. 8b). Next, to validate the hypothesis that triple sgRNA lentivirus has better RPE induction efficiency compared to the mix of individual lentiviruses (P+M+O), the experiment as shown in schematic FIG. 9a was carried out. It was observed that the expression of most of the RPE markers were slower with triple sgRNA transduced cells compared to P+M+O transduced cells on days 4, 10 and 18 (FIG. 9b). After replating the triple sgRNA cells on day 18, for the subsequent days, an increased expression of the activated transcription factors Pax6 and Mitf was observed, but the otx2 levels reduced as reported earlier (Ref. 11) (FIG. 10a). Pluripotency gene (Oct4-) decreased rapidly while the pigmentation genes (pMEL17, Tyr, TyrP1 and TyrP2) and mature markers (CRALBP, BEST1, RPE65 and PEDF) kept increasing throughout the period (FIG. 10a). Further examination of the cells in flow cytometry reveals similar phenomenon of high expression levels of pMEL17, Pax6 and Mitf, while the Otx2 protein expression levels were expressed at relatively consistent levels throughout the differentiation (FIG. 10b). More importantly, the morphology of pigmented clusters of cells in the 12-well plate was uniformly distributed throughout the well and the RPE signature cobblestone morphology was also present (FIG. 10c). These results confirms that the hypothesis of triple sgRNA design markedly improved RPE differentiation efficiency.

Next, in order to further optimize the protocol, different serum-free conditions were tested for the RPE maintenance media. The schematic of the differentiation protocol is shown in FIG. 11a. For this, the same protocol was maintained until day 18 and it was observed that the iPSC-CRISPR dCas9 SAM cells progressed gradually into RPE progenitor cells as observed with the increased expression of RPE-specific genes as observed earlier (FIG. 11b). After replating the RPE progenitor cells on day 18, the cells were maintained under two different serum-free formulations such as RPE maintenance media with, No FBS (0% FBS) or 5% KOSR. As a control, 5% FBS containing RPE was used as maintenance media. From this study, it is observed that 5% KOSR showed consistently higher RPE signature gene expression and the cells progressively matured into colonies comprising of a monolayer of pigmented polygonal cells and these pigmented cells within these colonies formed a cobble stone like sheets (FIGS. 11c and d). Immunostaining of cells enriched at day 21 of passage 1 iPSC-CRISPR dCas9 SAM derived RPE cells with mature tight junction markers ZO-1, N-Occludin, N-Cadherin and Bestrophin 1 showed that the cells were connected by tight junctions, properties which are highly characteristic of native RPE cells in vivo (FIG. 11e). Further immunostaining and flow cytometry analysis of activated transcription factors (Pax6, Mitf, Otx2) and pigmentation gene (pMEL17) showed higher and uniform expression, while pluripotency marker proteins (Oct4- and Tra-1-60) expression were low (FIGS. 11e and f). The optimized protocol disclosed herein was validated with hESC-CRISPR dCas9 SAM cells using triple sgRNA lentivirus and RPEM with 5% KOSR after replating the cells on day 18 and similar results was observed and RPE characteristics were as seen with iPSC CRIPSR dCas9-SAM cells (FIG. 12). This shows the robustness and reproducibility of this protocol with different pluripotent cells.

To further validate that activation of at least one (or all three) important to induce RPE generation, the RPE induction efficiency of triple sgRNA was compared with Mitf sgRNA only. Non-transduced cells was used as a control (i.e. No virus) as shown in FIG. 13a. It is evident from the data that, only triple sgRNA transduced cells generated RPE cells with characteristic RPE signature gene expression (FIG. 13b), pigmented cell clusters (FIG. 13c), typical cobble stone morphology (FIG. 13d) and protein expression (FIG. 13e). Overall, the present disclosure demonstrated that RPE cells can be rapidly and efficiently generated by activating only three transcription factors, Pax6, Mitf and Otx2 in pluripotent cells without the need for costly growth factors and small molecules.

Here, as a proof-of-concept the present study has demonstrated the activation of growth factors and cytokines (EPO, SCF, TPO, GM-CSF and G-CSF) using iPSC-CRISPR dCas9 SAM cells. For this, four different sgRNAs were designed for each of the growth factors/cytokines and constructed lentiviral vectors as mentioned previously (Table 4). For testing the activity of EPO expression, the lentiviruses of four different sgRNAs were produced and screened for the best performing guide in activating EPO expression. The iPSC-CRISPR dCas9 SAM cells were transduced with the unconcentrated lentivirus supernatant of the four sgRNAs individually and tested for their EPO gene expression using qPCR analysis on day 4 cells after transduction. It was found that g2 gave higher EPO gene expression as compared to the non-transduced control cells (FIG. 14a). Further, the spent media of the cells were collected from two wells of a 12-well plate on days 3 and 4 transduced with EPO_g2 and was stored in −20° C. Next, an Amicon Ultra-15 centrifugal filter was used with 10KDa cut-off membrane (EPO molecular weight: 21 KDa) to concentrate the EPO protein and the retentate washed with 1× PBS was used for ELISA assay. The collected data showed that EPO secreted from the iPSC-CRISPR dCas9 SAM cells transduced with EPO_g2 was detected by the commercial ELISA kit and the concentration was in the range of 47-51 IU/mL of EPO.

Similarly, the best performing sgRNA sequences for activating SCF, TPO, GM-CSF and G-CSF in iPSC-CRISPR dCas9 cells were screened (FIG. 15). The collected data showed, relatively lower expression of TPO, GM-CSF and G-CSF compared to SCF or EPO.

In the present study, EPO_g2 and SCF_g4 lentiviruses were concentrated according to previously described method. Transduction and selection of HEK-CRISPR dCas9 SAM cells were also shown. In the present disclosure, as shown in FIG. 16, the inventors have also stably transduced the EPO_g2 and SCF_g4 lentiviruses in HEK-CRISPR dCas9 SAM cells.

### REFERENCES

1. Li, L.; Hu, S.; Chen, X., Non-viral delivery systems for CRISPR/Cas9-based genome editing: Challenges and opportunities. Biomaterials 2018, 171, 207-218.

2. Glass, Z.; Lee, M.; Li, Y.; Xu, Q., Engineering the Delivery System for CRISPR-Based Genome Editing. Trends Biotechnol 2018, 36 (2), 173-185.

3. Konermann, S.; Brigham, M. D.; Trevino, A. E.; Joung, J.; Abudayyeh, O. O.; Barcena, C.; Hsu, P. D.; Habib, N.; Gootenberg, J. S.; Nishimasu, H.; Nureki, O.; Zhang, F., Genome-scale transcriptional activation by an engineered CRISPR-Cas9 complex. Nature 2015, 517 (7536), 583-588.

4. Yu, P.; Pan, G.; Yu, J.; Thomson, J. A., FGF2 sustains NANOG and switches the outcome of BMP4-induced human embryonic stem cell differentiation. Cell Stem Cell 2011, 8 (3), 326-34.

5. Meyer, J. S.; Shearer, R. L.; Capowski, E. E.; Wright, L. S.; Wallace, K. A.; McMillan, E. L.; Zhang, S. C.; Gamm, D. M., Modeling early retinal development with human embryonic and induced pluripotent stem cells. Proc Natl Acad Sci U S A 2009, 106 (39), 16698-703.

6. Radeke, M. J.; Peterson, K. E.; Johnson, L. V.; Anderson, D. H., Disease susceptibility of the human macula: differential gene transcription in the retinal pigmented epithelium/choroid. Exp Eye Res 2007, 85 (3), 366-80.

7. Amram, B.; Cohen-Tayar, Y.; David, A.; Ashery-Padan, R., The retinal pigmented epithelium—from basic developmental biology research to translational approaches. Int J Dev Biol 2017, 61 (3-4-5), 225-234.

8. Luo, M.; Chen, Y., Application of stem cell-derived retinal pigmented epithelium in retinal degenerative diseases: present and future. Int J Ophthalmol 2018, 11 (1), 150-159.

9. da Cruz, L.; Fynes, K.; Georgiadis, 0.; Kerby, J.; Luo, Y. H.; Ahmado, A.; Vernon, A.; Daniels, J. T.; Nommiste, B.; Hasan, S. M.; Gooljar, S. B.; Carr, A. F.; Vugler, A.; Ramsden, C. M.; Bictash, M.; Fenster, M.; Steer, J.; Harbinson, T.; Wilbrey, A.; Tufail, A.; Feng, G.; Whitlock, M.; Robson, A. G.; Holder, G. E.; Sagoo, M. S.; Loudon, P. T.; Whiting, P.; Coffey, P. J., Phase 1 clinical study of an embryonic stem cell-derived retinal pigment epithelium patch in age-related macular degeneration. Nat Biotechnol 2018, 36 (4), 328-337.

10. Buchholz, D. E.; Hikita, S. T.; Rowland, T. J.; Friedrich, A. M.; Hinman, C. R.; Johnson, L. V.; Clegg, D. O., Derivation of functional retinal pigmented epithelium from induced pluripotent stem cells. Stem Cells 2009, 27 (10), 2427-34.

11. Buchholz, D. E.; Pennington, B. O.; Croze, R. H.; Hinman, C. R.; Coffey, P. J.; Clegg, D. O., Rapid and efficient directed differentiation of human pluripotent stem cells into retinal pigmented epithelium. Stem Cells Transl Med 2013, 2 (5), 384-93.

12. Idelson, M.; Alper, R.; Obolensky, A.; Ben-Shushan, E.; Hemo, I.; Yachimovich-Cohen, N.; Khaner, H.; Smith, Y.; Wiser, O.; Gropp, M.; Cohen, M. A.; Even-Ram, S.; Berman-Zaken, Y.; Matzrafi, L.; Rechavi, G.; Banin, E.; Reubinoff, B., Directed differentiation of human embryonic stem cells into functional retinal pigment epithelium cells. Cell Stem Cell 2009, 5 (4), 396-408.

13. Maruotti, J.; Sripathi, S. R.; Bharti, K.; Fuller, J.; Wahlin, K. J.; Ranganathan, V.; Sluch, V. M.; Berlinicke, C. A.; Davis, J.; Kim, C.; Zhao, L.; Wan, J.; Qian, J.; Corneo, B.; Temple, S.; Dubey, R.; Olenyuk, B. Z.; Bhutto, I.; Lutty, G. A.; Zack, D. J., Small-molecule-directed, efficient generation of retinal pigment epithelium from human pluripotent stem cells. Proceedings of the National Academy of Sciences 2015, 112 (35), 10950.

14. Osakada, F.; Jin, Z. B.; Hirami, Y.; Ikeda, H.; Danjyo, T.; Watanabe, K.; Sasai, Y.; Takahashi, M., In vitro differentiation of retinal cells from human pluripotent stem cells by small-molecule induction. J Cell Sci 2009, 122 (Pt 17), 3169-79.

15. Zahabi, A.; Shahbazi, E.; Ahmadieh, H.; Hassani, S. N.; Totonchi, M.; Taei, A.; Masoudi, N.; Ebrahimi, M.; Aghdami, N.; Seifinejad, A.; Mehrnejad, F.; Daftarian, N.; Salekdeh, G. H.; Baharvand, H., A new efficient protocol for directed differentiation of retinal pigmented epithelial cells from normal and retinal disease induced pluripotent stem cells. Stem Cells Dev 2012, 21 (12), 2262-72.

16. Zhu, Y.; Carido, M.; Meinhardt, A.; Kurth, T.; Karl, M. O.; Ader, M.; Tanaka, E. M., Three-Dimensional Neuroepithelial Culture from Human Embryonic Stem Cells and Its Use for Quantitative Conversion to Retinal Pigment Epithelium. PLOS ONE 2013, 8 (1), e54552.

17. Choudhary, P.; Booth, H.; Gutteridge, A.; Surmacz, B.; Louca, I.; Steer, J.; Kerby, J.; Whiting, P. J., Directing Differentiation of Pluripotent Stem Cells Toward Retinal Pigment Epithelium Lineage. Stem Cells Transl Med 2017, 6 (2), 490-501.

18. Geng, Z.; Walsh, P. J.; Truong, V.; Hill, C.; Ebeling, M.; Kapphahn, R. J.; Montezuma, S. R.; Yuan, C.; Roehrich, H.; Ferrington, D. A.; Dutton, J. R., Generation of retinal pigmented epithelium from iPSCs derived from the conjunctiva of donors with and without age related macular degeneration. PLOS ONE 2017, 12 (3), e0173575.

19. Zhang, K.; Liu, G.-H.; Yi, F.; Montserrat, N.; Hishida, T.; Esteban, C. R.; lzpisua Belmonte, J. C., Direct conversion of human fibroblasts into retinal pigment epithelium-like cells by defined factors. Protein & cell 2014, 5 (1), 48-58.

20. D′3 ssio, A. C.; Fan, Z. P.; Wert, K. J.; Baranov, P.; Cohen, M. A.; Saini, J. S.; Cohick, E.; Charniga, C.; Dadon, D.; Hannett, N. M.; Young, M. J.; Temple, S.; Jaenisch, R.; Lee, T. I.; Young, R. A., A Systematic Approach to Identify Candidate Transcription Factors that Control Cell Identity. Stem Cell Reports 2015, 5 (5), 763-775.

### Applications

Embodiments of the methods disclosed herein provide a fast, efficient and cheap way of programming a cell. Embodiments of the disclosed methods also seek to overcome the problems relating to methods of altering a differentiation status of a cell (by expressing genes and/or proteins in the cell).

As discussed in the background section, convention methods of expressing proteins are rife with problems. As an alternative, the present inventors found a simple but surprisingly effective and easy methods that are discussed in further detail in the present disclosure. For example, in various embodiments, the methods as describe herein may use suspension of human cells (such as human embryonic kidney (HEK) cells) instead of traditional host cells (such as CHO or bacterial cells) to overcome one or more of the limitations known in the art. The methods as described herein also advantageously capable of producing stable producer lines and very cost effective as the cost of media for culturing human cells (such as HEK cells) are lower than the cost of media for culturing traditional host cells (such as CHO or bacterial cells).

Furthermore, the use of CRISPR activation method to activate the genes to produce proteins endogenously, instead of recombinant DNA, also overcame many of the limitations known in the art.

Advantageously, the present disclosure demonstrates a simple method of differentiating a stem cell to a mature/differentiated cell (such as retinal pigment epithelial cells). In particular, the method advantageously only uses minimal set of transcription factors. For example, when the method differentiates human pluripotent stem cells to retinal pigment epithelium cells using CRISPR/dCas9-SAM mediated activation, minimal set of transcription factors is required.

Even more advantageously, the present disclosure demonstrates a method of altering the differentiation status of a cell without the use of growth factors and/or small molecules. That is, the present method is free of the use of growth factors and/or small molecules (either in any of the steps or in the solution/media used). This feature reduces the total costs of running the method and, thus, is a cost-effective method. For example, in one of the embodiment of the present disclosure, activation of one or more (such as three) key transcription factors (such as PAX6, MITF, and OTX2) is sufficient to generate retinal pigment epithelial cells without the need for costly growth factors or small molecules. The protocols are also free of laborious differentiation steps.

For one of the embodiments of the present disclosure, the inventors have generated unique sgRNA sequences that can specifically activate PAX6, MITF and OTX2 genes with higher fold change respectively. When one or more of these transcription factors are used to generate retinal pigment epithelial cells, the method advantageously generates desired cell in a short time period. This is illustrated in the appearance of cobblestone morphology of highly pure RPE cell cultures (>96% PMEL17) within 40 days of activation of transcription factors (TFs).

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A method of altering a differentiation status of a cell, the method comprising:
modulating the expression of one or more differentiation factors with a nuclease-deactivated Cas9 (dCas9) fusion protein, the dCas9 fusion protein comprising dCas9 and an effector comprising a transcriptional regulator, optionally the transcription regulator is a transcriptional activator.

2. The method of claim 1, the method further comprising:
providing a guide RNA (gRNA) in the cell, wherein the gRNA is capable of guiding the dCas9 fusion protein to a target site that is/that is in proximity of a promoter region of the one or more differentiation factors to allow the dCas9 fusion protein to modulate the expression of the one or more differentiation factors, optionally wherein the target site that is/that is in proximity of the promoter region is within an about −300 base pairs (bp) to about +5 bp window of the promoter region.

3. (canceled)

4. The method of claim 1, the method further comprising:
providing an activator module comprising a RNA-binding protein capable of binding to the gRNA, optionally wherein the RNA-binding protein comprises MS2 coat protein (MCP), optionally wherein the activator module further comprises one or more transcriptional activators, optionally the transcriptional activator is selected from the group consisting of VP64, p65, HSF1, Rta and combinations thereof, optionally wherein the activator module comprises p65 and/or HSF1.

5.-6. (canceled)

7. The method of claim 1, wherein the dCas9 fusion protein comprises VP64 and optionally, p65 and/or Rta, or the method further comprising expressing the dCas9 fusion protein, optionally a dCas9-VP64 fusion protein and/or a dCas9-VP64-p65-Rta (dCas9-VPR) fusion protein, prior to the modulating step, or the method comprises modulating the expression of one or more differentiation factors with a CRISPR/dCas9 synergistic activation mediators (CRISPR/dCas9-SAM) complex/dCas9 ribonucleoprotein complex (e.g. a complex comprising the dCas9 fusion protein)/dCas9-VP64/dCas9-VPR/dCas9-VP64 and MS2-P65-HSF1.

8.-9. (canceled)

10. The method of claim 1, wherein the one or more differentiation factors comprises transcription factors, optionally wherein the cell is a stem cell, stem cell-like cell, a progenitor cell or a precursor cell, optionally the cell comprises one that is selected from the group consisting of embryonic stem cell (e.g. hESC3), adult stem cell, induced pluripotent stem cell (iPSC), mesenchymal stem cell (MSC), human embryonic kidney cell (HEK293) and the like.

11. (canceled)

12. The method of claim 1, wherein the method is a method of differentiating a cell, optionally the one or more differentiation factors influence an expression of a neuroprogenitor gene and/or a retinal pigment epithelium (RPE)-associated gene, optionally the RPE-associated gene comprises a gene associated with a mature RPE/RPE specific mature gene, a gene associated with pigmentation/RPE specific pigmentation gene or early eye field gene.

13. (canceled)

14. The method of claim 1, wherein the one or more differentiation factors is selected from the group consisting of PAX6, MITF, OTX2 and combinations thereof, optionally the one or more differentiation factors is selected from the group consisting of LHX2, RAX2, Tyrosinase, CRALBP, BEST1, RPE65, PEDF, pme117, PYR, Trypl, Tryp2, CRX and combinations thereof.

15. (canceled)

16. The method of claim 1, wherein the cell produced from the method expresses premelanosome marker 17 (PMEL17), optionally the expression of PMEL17 in the produced cell is at least about 50%, or wherein the cell produced from the method expresses Pax6, optionally the cell is a neuroprogenitor cell.

17.-19. (canceled)

20. The method of claim 1, wherein the method is free of modulating the expression of a transcription activator selected from the group consisting of: cMyc, Klf4, Nrl, Crx, Rax, LHX2, SIX3, SOX9, GLIS3, FOXD1, ZNF92 , C11or19 and combinations thereof directly via the dCas9 fusion protein, or the method is free of the use of a gRNA specific to a target site that is/that is in proximity of a promoter region of: cMyc, Klf4, Nrl, Crx, Rax, LHX2, SIX3, SOX9, GLIS3, FOXD1, ZNF92, C11orf9 and combinations thereof, or the method is free of exogenous growth factor, free of inducible system, and/or is free of whole exogenous nucleic acid, optionally wherein modulating the expression of one or more differentiation factors comprises an endogenous activation of the one or more differentiation factors.

21.-27. (canceled)

28. A guide RNA (gRNA) to a target site that is or that is in proximity of the promoter region of one or more differentiation factors to modulate the expression of the one or more differentiation factors, wherein the gRNA is configured to guide a fusion protein selected from the group consisting of dCas9 fusion protein, CRISPR/dCas9 synergistic activation mediators (CRISPR/dCas9-SAM) complex, dCas9 ribonucleoprotein complex, dCas9-VP64, dCas9-VPR, dCas9-VP64, and MS2-P65-HSF1, optionally wherein the gRNA is a single/short gRNA (sgRNA).

29. The gRNA of claim 28, wherein at least a portion of the guide RNA is capable of binding to the target site/target genomic locus that is in an about −300 base pairs (bp) to about +5 bp window of the promoter region of one or more differentiation factors selected from the group consisting of PAX6, MITF, OTX2, and combinations thereof.

30. The gRNA of claim 28, wherein the gRNA has about 15 bp to about 25 bp, optionally wherein the gRNA has at least about 80% identity with a sequence selected the group consisting of SEQ ID NO: 1 (AATGTGTGTGTGCCGGCGCC), SEQ ID NO: 2 (GCCAGCACACCTATGCTGAT), SEQ ID NO: 3 (GCTTCGCTAATGGGCCAGTG), SEQ ID NO: 4 (ACAATAAAATGGGCTGTCAG), SEQ ID NO: 5 (GAGTGAGAGATAAAGAGTGT), SEQ ID NO: 6 (CGGGCCGAACTACAGATCCC), SEQ ID NO: 7 (CCAAACAGGAGTTGCACTAG), SEQ ID NO: 8 (AGCTGTAGTTTTCGTGGGAG), SEQ ID NO: 9 (GCGGGGGAGAGGCAACGTGG), SEQ ID NO: 10 (CTGTACCCTTGAAGCAAGTG), SEQ ID NO: 11 (GAACATTCTGGTAATGTCGG), SEQ ID NO: 12 (GCGTCAAAAAGTTGCCAGAG), SEQ ID NO: 13 (AACAGGCCGCTGCTGCACGG), SEQ ID NO: 14 (GATTGACACATCTAAGCCAG), SEQ ID NO: 15 (TAAAAACACACAACAGGGGG), SEQ ID NO: 76 (GGGGTGGCCCAGGGACTCTG), SEQ ID NO: 77 (TGTGCGTGAGGGGTCGCCAG), SEQ ID NO: 78 (GCCCCTGCTCTGACCCCGGG), SEQ ID NO: 79 (GGAGAGGCTGTGTGCGTGAG), SEQ ID NO: 80 (GAACTGTATAAAAGCGCCGG), SEQ ID NO: 81 (CCTAATCTGCCAAACTTCTG), SEQ ID NO: 82 (GAGGCGTGTCCGGAGCAGGC), SEQ ID NO: 83 (GGTAGGCGAGAAGCAGGCAA), SEQ ID NO: 84 (TCCTTCCCTTCCGGAGCCCG), SEQ ID NO: 85 (GAGCCACCAGACACTGGTGA), SEQ ID NO: 86 (CCCTATCCAAATCTTCTCCG), SEQ ID NO: 87 (ACTTCTGCCCAATCAGAGAA), SEQ ID NO: 88 (AAGAGAAGGCGTCACTTCCG), SEQ ID NO: 89 (AGCAGGTCATACGCCTGCCT), SEQ ID NO: 90 (AAGAGCTCTTAAATACACAG), SEQ ID NO: 91 (GTGACCACAAAATGCCAGGG), SEQ ID NO: 92 (CGGGGGAACTACCTGAACTG), SEQ ID NO: 93 (GGCCCTTATCAGCCACACAT), SEQ ID NO: 94 (AGGCTCACCGTTCCCATGTG), SEQ ID NO: 95 (GTGTCCAAGACAATGCAGGG), SEQ ID NO: 96 (GGGCAAGGCGACGTCAAAGG), SEQ ID NO: 97 (GCGAAAGTTTTGTGAAATTG), SEQ ID NO: 98 (GGGGGGCAAGGCGACGTCAA), and SEQ ID NO: 99 (CACCAAATTTGCATAAATCC).

31.-32. (canceled)

33. The gRNA of claim 28, wherein the gRNA is provided in a set comprising at least two of the gRNA, wherein the gRNA is selected from the group consisting of: a gRNA that is specific to a target site that is/that is in proximity of the promoter region of PAX6, a gRNA that is specific to a target site that is/that is in proximity of the promoter region of MITF and a gRNA that is specific to a target site that is/that is in proximity of the promoter region of OTX2.

34. The gRNA of claim 28, wherein the gRNA is cloned with a oligonucleotide/primer having at least about 80% with a sequence selected from Table 2 below:
TABLE 2

| | | SEQ |
|---|---|---|
| | | ID |
| Name | Sequence | NO. |
| Pax6_1_Fwd | CACCGACAATAAAATGGGCTGTCAG | 16 |
| Pax6_1_Rev | AAACCTGACAGCCCATTTTATTGTC | 17 |
| Pax6_2_Fwd | CACCGGAGTGAGAGATAAAGAGTGT | 18 |
| Pax6_2_Rev | AAACACACTCTTTATCTCTCACTCC | 19 |
| Pax6_3_Fwd | CACCGGCCAGCACACCTATGCTGAT | 20 |
| Pax6_3_Rev | AAACATCAGCATAGGTGTGCTGGCC | 21 |
| Pax6_4_Fwd | CACCGAATGTGTGTGTGCCGGCGCC | 22 |
| Pax6_4_Rev | AAACGGCGCCGGCACACACACATTC | 23 |
| Pax6_5_Fwd | CACCGGCTTCGCTAATGGGCCAGTG | 24 |
| Pax6_5_Rev | AAACCACTGGCCCATTAGCGAAGCC | 25 |
| MITF_1_Fwd | CACCGCGGGCCGAACTACAGATCCC | 26 |
| MITF_1_Rev | AAACGGGATCTGTAGTTCGGCCCGC | 27 |
| MITF_2_Fwd | CACCGCCAAACAGGAGTTGCACTAG | 28 |
| MITF_2_Rev | AAACCTAGTGCAACTCCTGTTTGGC | 29 |
| MITF_3_Fwd | CACCGGCGGGGGAGAGGCAACGTGG | 30 |
| MITF_3_Rev | AAACCCACGTTGCCTCTCCCCCGCC | 31 |
| MITF_4_Fwd | CACCGAGCTGTAGTTTTCGTGGGAG | 32 |
| MITF_4_Rev | AAACCTCCCACGAAAACTACAGCTC | 33 |
| MITF_5_Fwd | CACCGCTGTACCCTTGAAGCAAGTG | 34 |
| MITF_5_Rev | AAACCACTTGCTTCAAGGGTACAGC | 35 |
| OTX2_1_Fwd | CACCGGCGTCAAAAAGTTGCCAGAG | 36 |
| OTX2_1_Rev | AAACCTCTGGCAACTTTTTGACGCC | 37 |
| OTX2_2_Fwd | CACCGGAACATTCTGGTAATGTCGG | 38 |
| OTX2_2_Rev | AAACCCGACATTACCAGAATGTTCC | 39 |
| OTX2_3_Fwd | CACCGTAAAAACACACAACAGGGGG | 40 |
| OTX2_3_Rev | AAACCCCCCTGTTGTGTGTTTTTAC | 41 |
| OTX2_4_Fwd | CACCGAACAGGCCGCTGCTGCACGG | 42 |
| OTX2_4_Rev | AAACCCGTGCAGCAGCGGCCTGTTC | 43 |
| OTX2_5_Fwd | CACCGGATTGACACATCTAAGCCAG | 44 |
| OTX2_5_Rev | AAACCTGGCTTAGATGTGTCAATCC | 45 |

35. The gRNA of claim 28, comprised in a composition comprising:
a dCas9 fusion protein, the dCas9 fusion protein comprising dCas9 and an effector; and
optionally an activator module comprising a RNA-binding protein capable of binding to the gRNA, further optionally wherein the RNA-binding protein comprises MS2 coat protein (MCP).

36.-37. (canceled)

38. A method of treating a disease, the method comprising transplanting, to a patient in need thereof, (i) a cell comprising a dCas9 fusion protein that is configured to modulate the expression of one or more differentiation factors, the dCas9 fusion protein comprising dCas9 and an effector, or progenies thereof, or (ii) a cell that has a second differentiation status (or its progenies thereof) that was differentiated from a cell having a first differentiation status.

39. The method of claim 38, wherein the disease is an eye disease/disorder, optionally wherein the eye disease/disorder is selected from the group consisting of macular degeneration, acute macular degeneration (AMD), atrophic age-related macular degeneration (atrophic AMD), dry age-related macular degeneration (Dry-type AMD), retinitis pigmentosa (RP), Stargardt's disease, and myopia.

40. The method of claim 38, wherein the cell in (i) comprises a guide RNA (gRNA) capable of guiding the dCas9 fusion protein to a target site that is/that is in proximity of the promoter region of the one or more differentiation factors to allow the dCas9 fusion protein to modulate the expression of the one or more differentiation factors.

41. The method of claim 38, wherein the cell in (ii) has the second differentiation status is devoid of a dCas9 fusion protein or a CRISPR/dCas9-SAM complex.


SEQUENCE LISTING (99 sequences)

SEQ ID NO: 1 - DNA, 20 bp, Artificial sequence
  Synthetic sequence
aatgtgtgtgtgccggcgcc

SEQ ID NO: 2 - DNA, 20 bp, Artificial sequence
  Synthetic sequence
gccagcacacctatgctgat

SEQ ID NO: 3 - DNA, 20 bp, Artificial sequence
  Synthetic Sequence
gcttcgctaatgggccagtg

SEQ ID NO: 4 - DNA, 20 bp, Artificial sequence
  Synthetic sequence
acaataaaatgggctgtcag

SEQ ID NO: 5 - DNA, 20 bp, Artificial sequence
  Synthetic sequence
gagtgagagataaagagtgt

SEQ ID NO: 6 - DNA, 20 bp, Artificial sequence
  Synthetic sequence
cgggccgaactacagatccc

SEQ ID NO: 7 - DNA, 20 bp, Artificial sequence
  Synthetic sequence
ccaaacaggagttgcactag

SEQ ID NO: 8 - DNA, 20 bp, Artificial sequence
  Synthetic sequence
agctgtagttttcgtgggag

SEQ ID NO: 9 - DNA, 20 bp, Artificial sequence
  Synthetic sequence
gcgggggagaggcaacgtgg

SEQ ID NO: 10 - DNA, 20 bp, Artificial sequence
  Synthetic sequence
ctgtacccttgaagcaagtg

SEQ ID NO: 11 - DNA, 20 bp, Artificial sequence
  Synthetic sequence
gaacattctggtaatgtcgg

SEQ ID NO: 12 - DNA, 20 bp, Artificial sequence
  Synthetic sequence
gcgtcaaaaagttgccagag

SEQ ID NO: 13 - DNA, 20 bp, Artificial sequence
  Synthetic sequence
aacaggccgctgctgcacgg

SEQ ID NO: 14 - DNA, 20 bp, Artificial sequence
  Synthetic sequence
gattgacacatctaagccag

SEQ ID NO: 15 - DNA, 20 bp, Artificial sequence
  Synthetic sequence
taaaaacacacaacaggggg

SEQ ID NO: 16 - DNA, 25 bp, Artificial sequence
  Synthetic sequence
caccgacaataaaatgggctgtcag

SEQ ID NO: 17 - DNA, 25 bp, Artificial sequence
  Synthetic sequence
aaacctgacagcccattttattgtc

SEQ ID NO: 18 - DNA, 25 bp, Artificial sequence
  Synthetic sequence
caccggagtgagagataaagagtgt

SEQ ID NO: 19 - DNA, 25 bp, Artificial sequence
  Synthetic sequence
aaacacactctttatctctcactcc

SEQ ID NO: 20 - DNA, 25 bp, Artificial sequence
  Synthetic sequence
caccggccagcacacctatgctgat

SEQ ID NO: 21 - DNA, 25 bp, Artificial sequence
  Synthetic sequence
aaacatcagcataggtgtgctggcc

SEQ ID NO: 22 - DNA, 25 bp, Artificial sequence
  Synthetic sequence
caccgaatgtgtgtgtgccggcgcc

SEQ ID NO: 23 - DNA, 25 bp, Artificial sequence
  Synthetic sequence
aaacggcgccggcacacacacattc

SEQ ID NO: 24 - DNA, 25 bp, Artificial sequence
  Synthetic sequence
caccggcttcgctaatgggccagtg

SEQ ID NO: 25 - DNA, 25 bp, Artificial sequence
  Synthetic sequence
aaaccactggcccattagcgaagcc

SEQ ID NO: 26 - DNA, 25 bp, Artificial sequence
  Synthetic sequence
caccgcgggccgaactacagatccc

SEQ ID NO: 27 - DNA, 25 bp, Artificial sequence
  Synthetic sequence
aaacgggatctgtagttcggcccgc

SEQ ID NO: 28 - DNA, 25 bp, Artificial sequence
  Synthetic sequence
caccgccaaacaggagttgcactag

SEQ ID NO: 29 - DNA, 25 bp, Artificial sequence
  Synthetic sequence
aaacctagtgcaactcctgtttggc

SEQ ID NO: 30 - DNA, 25 bp, Artificial sequence
  Synthetic sequence
caccggcgggggagaggcaacgtgg

SEQ ID NO: 31 - DNA, 25 bp, Artificial sequence
  Synthetic sequence
aaacccacgttgcctctcccccgcc

SEQ ID NO: 32 - DNA, 25 bp, Artificial sequence
  Synthetic sequence
caccgagctgtagttttcgtgggag

SEQ ID NO: 33 - DNA, 25 bp, Artificial sequence
  Synthetic sequence
aaacctcccacgaaaactacagctc

SEQ ID NO: 34 - DNA, 25 bp, Artificial sequence
  Synthetic sequence
caccgctgtacccttgaagcaagtg

SEQ ID NO: 35 - DNA, 25 bp, Artificial sequence
  Synthetic sequence
aaaccacttgcttcaagggtacagc

SEQ ID NO: 36 - DNA, 25 bp, Artificial sequence
  Synthetic sequence
caccggcgtcaaaaagttgccagag

SEQ ID NO: 37 - DNA, 25 bp, Artificial sequence
  Synthetic sequence
aaacctctggcaactttttgacgcc

SEQ ID NO: 38 - DNA, 25 bp, Artificial sequence
  Synthetic sequence
caccggaacattctggtaatgtcgg

SEQ ID NO: 39 - DNA, 25 bp, Artificial sequence
  Synthetic sequence
aaacccgacattaccagaatgttcc

SEQ ID NO: 40 - DNA, 25 bp, Artificial sequence
  Synthetic sequence
caccgtaaaaacacacaacaggggg

SEQ ID NO: 41 - DNA, 25 bp, Artificial sequence
  Synthetic sequence
aaacccccctgttgtgtgtttttac

SEQ ID NO: 42 - DNA, 25 bp, Artificial sequence
  Synthetic sequence
caccgaacaggccgctgctgcacgg

SEQ ID NO: 43 - DNA, 25 bp, Artificial sequence
  Synthetic sequence
aaacccgtgcagcagcggcctgttc

SEQ ID NO: 44 - DNA, 25 bp, Artificial sequence
  Synthetic sequence
caccggattgacacatctaagccag

SEQ ID NO: 45 - DNA, 25 bp, Artificial sequence
  Synthetic sequence
aaacctggcttagatgtgtcaatcc

SEQ ID NO: 46 - DNA, 19 bp, Artificial sequence
  Synthetic sequence
cagtgcccgaaacccacac

SEQ ID NO: 47 - DNA, 20 bp, Artificial sequence
  Synthetic sequence
ggagacccagcagcctcaaa

SEQ ID NO: 48 - DNA, 21 bp, Artificial sequence
  Synthetic sequence
cgtccgtcttaacttctgtgc

SEQ ID NO: 49 - DNA, 21 bp, Artificial sequence
  Synthetic sequence
aggttggtaagagtcgtttgt

SEQ ID NO: 50 - DNA, 21 bp, Artificial sequence
  Synthetic sequence
tcccaggaggcttggagaccc

SEQ ID NO: 51 - DNA, 21 bp, Artificial sequence
  Synthetic sequence
ctccccaagtcctgagcgtgc

SEQ ID NO: 52 - DNA, 21 bp, Artificial sequence
  Synthetic sequence
agttccgagaggcatagaagg

SEQ ID NO: 53 - DNA, 21 bp, Artificial sequence
  Synthetic sequence
taagcagattggtttgtccat

SEQ ID NO: 54 - DNA, 21 bp, Artificial sequence
  Synthetic sequence
ctcggtggtgtctttgtcaac

SEQ ID NO: 55 - DNA, 21 bp, Artificial sequence
  Synthetic sequence
acttttgcatctgcatgggtc

SEQ ID NO: 56 - DNA, 21 bp, Artificial sequence
  Synthetic sequence
cccagttcatgcaacagagag

SEQ ID NO: 57 - DNA, 18 bp, Artificial sequence
  Synthetic sequence
gcagagggaagggtggtg

SEQ ID NO: 58 - DNA, 22 bp, Artificial sequence
  Synthetic sequence
gtgtagccttcttccaactcag

SEQ ID NO: 59 - DNA, 24 bp, Artificial sequence
  Synthetic sequence
gttcctcattaccaaatagcatcc

SEQ ID NO: 60 - DNA, 23 bp, Artificial sequence
  Synthetic sequence
gattccactctaataagcccaaa

SEQ ID NO: 61 - DNA, 20 bp, Artificial sequence
  Synthetic sequence
ttccaagcactgagcgacat

SEQ ID NO: 62 - DNA, 24 bp, Artificial sequence
  Synthetic sequence
ctcagaccaacttggctacagcta

SEQ ID NO: 63 - DNA, 22 bp, Artificial sequence
  Synthetic sequence
cagcacaaaaagaccaaccaaa

SEQ ID NO: 64 - DNA, 19 bp, Artificial sequence
  Synthetic sequence
tgatggctgtggtccttgc

SEQ ID NO: 65 - DNA, 20 bp, Artificial sequence
  Synthetic sequence
cagtgactgctgctatgtgg

SEQ ID NO: 66 - DNA, 24 bp, Artificial sequence
  Synthetic sequence
tatcaccttaaccagcctttcatc

SEQ ID NO: 67 - DNA, 21 bp, Artificial sequence
  Synthetic sequence
gggtccagaatcttgccaatg

SEQ ID NO: 68 - DNA, 19 bp, Artificial sequence
  Synthetic sequence
tagaaccatcagcgccgtc

SEQ ID NO: 69 - DNA, 21 bp, Artificial sequence
  Synthetic sequence
tgagtgtagtgtgtatgttgg

SEQ ID NO: 70 - DNA, 20 bp, Artificial sequence
  Synthetic sequence
cacgctgcccaagtatgatg

SEQ ID NO: 71 - DNA, 20 bp, Artificial sequence
  Synthetic sequence
ccaggacagttgaggagagg

SEQ ID NO: 72 - DNA, 24 bp, Artificial sequence
  Synthetic sequence
cctgattcatacccatcagaaccc

SEQ ID NO: 73 - DNA, 24 bp, Artificial sequence
  Synthetic sequence
caccacactcagaactacaccatc

SEQ ID NO: 74 - DNA, 22 bp, Artificial sequence
  Synthetic sequence
agcaagagcacaagaggaagag

SEQ ID NO: 75 - DNA, 24 bp, Artificial sequence
  Synthetic sequence
gagcacagggtactttattgatgg

SEQ ID NO: 76 - DNA, 20 bp, Artificial sequence
  Synthetic sequence
ggggtggcccagggactctg

SEQ ID NO: 77 - DNA, 20 bp, Artificial sequence
  Synthetic sequence
tgtgcgtgaggggtcgccag

SEQ ID NO: 78 - DNA, 20 bp, Artificial sequence
  Synthetic sequence
gcccctgctctgaccccggg

SEQ ID NO: 79 - DNA, 20 bp, Artificial sequence
  Synthetic sequence
ggagaggctgtgtgcgtgag

SEQ ID NO: 80 - DNA, 20 bp, Artificial sequence
  Synthetic sequence
gaactgtataaaagcgccgg

SEQ ID NO: 81 - DNA, 20 bp, Artificial sequence
  Synthetic sequence
cctaatctgccaaacttctg

SEQ ID NO: 82 - DNA, 20 bp, Artificial sequence
  Synthetic sequence
gaggcgtgtccggagcaggc

SEQ ID NO: 83 - DNA, 20 bp, Artificial sequence
  Synthetic sequence
ggtaggcgagaagcaggcaa

SEQ ID NO: 84 - DNA, 20 bp, Artificial sequence
  Synthetic sequence
tccttcccttccggagcccg

SEQ ID NO: 85 - DNA, 20 bp, Artificial sequence
  Synthetic sequence
gagccaccagacactggtga

SEQ ID NO: 86 - DNA, 20 bp, Artificial sequence
  Synthetic sequence
ccctatccaaatcttctccg

SEQ ID NO: 87 - DNA, 20 bp, Artificial sequence
  Synthetic sequence
acttctgcccaatcagagaa

SEQ ID NO: 88 - DNA, 20 bp, Artificial sequence
  Synthetic sequence
aagagaaggcgtcacttccg

SEQ ID NO: 89 - DNA, 20 bp, Artificial sequence
  Synthetic sequence
agcaggtcatacgcctgcct

SEQ ID NO: 90 - DNA, 20 bp, Artificial sequence
  Synthetic sequence
aagagctcttaaatacacag

SEQ ID NO: 91 - DNA, 20 bp, Artificial sequence
  Synthetic sequence
gtgaccacaaaatgccaggg

SEQ ID NO: 92 - DNA, 20 bp, Artificial sequence
  Synthetic sequence
cgggggaactacctgaactg

SEQ ID NO: 93 - DNA, 20 bp, Artificial sequence
  Synthetic sequence
ggcccttatcagccacacat

SEQ ID NO: 94 - DNA, 20 bp, Artificial sequence
  Synthetic sequence
aggctcaccgttcccatgtg

SEQ ID NO: 95 - DNA, 20 bp, Artificial sequence
  Synthetic sequence
gtgtccaagacaatgcaggg

SEQ ID NO: 96 - DNA, 20 bp, Artificial sequence
  Synthetic sequence
gggcaaggcgacgtcaaagg

SEQ ID NO: 97 - DNA, 20 bp, Artificial sequence
  Synthetic sequence
gcgaaagttttgtgaaattg

SEQ ID NO: 98 - DNA, 20 bp, Artificial sequence
  Synthetic sequence
ggggggcaaggcgacgtcaa

SEQ ID NO: 99 - DNA, 20 bp, Artificial sequence
  Synthetic sequence
caccaaatttgcataaatcc